(19) United States
(12) Patent Application Publication
(10) Pub. No.: US 2013/0267437 A1
(43) Pub. Date: Oct. 10, 2013

(54) USE OF SPECIFIC GENES OR THEIR ENCODED PROTEINS FOR A PROGNOSIS METHOD OF CLASSIFIED LUNG CANCER

(71) Applicants: Sophie Pison-Rousseaux, Saint Martin D'Uriage (FR); Saadi Khochbin, Meylan (FR)

(72) Inventors: Sophie Pison-Rousseaux, Saint Martin D'Uriage (FR); Saadi Khochbin, Meylan (FR)

(73) Assignee: UNIVERSITE JOSEPH FOURIER, Grenoble Cedex 09 (FR)

(21) Appl. No.: 13/880,419

(22) PCT Filed: Oct. 20, 2011
(86) PCT No.: PCT/EP2011/068377

§ 371 Date: Jun. 26, 2013

(30) Foreign Application Priority Data
Oct. 20, 2010 (EP) 10306143.8

(51) Int. Cl. C12Q 1/68; G01N 33/68

(52) U.S. Cl. CPC C12Q 1/6886; G01N 33/6884
USPC 506/9; 506/16; 506/18

(57) ABSTRACT

At least one element for the implementation of a prognosis method, preferably in vitro, of the survival rate of a patient afflicted by a lung cancer, the lung cancer being previously classified as a lung tumour selected from squamous cell carcinoma (SQC), adenocarcinoma (ADK), large cell neuro-endocrine tumours (LCNE) and basaloid tumours (BAS).

The present invention relates to a prognosis method of lung cancer aby using specific genes.

Lung cancer is a disease of uncontrolled cell growth in tissues of the lung. This growth may lead to metastasis, which is the invasion of adjacent tissues and infiltration beyond the lungs. The vast majority of primary lung cancers are carcinomas of the lung, derived from epithelial cells. Lung cancer, the most common cause of cancer-related death in men and women, is responsible for 1.3 million deaths worldwide annually, as of 2004. The most common symptoms are shortness of breath, coughing (including coughing up blood), and weight loss.

Due to the high prevalence of this type of tumors, there is a need to efficiently diagnose lung cancer. Moreover, it is important to propose a prognosis method that allows the pathologist to determine, when a patient is afflicted by lung tumors, the survival rate during a short and a long period, and consequently to propose an adapted therapy.

Presently, several clinical and pathological parameters help defining the prognosis, including histological subtypes, TNM stages (tumour size, presence of tumour cells in lymph nodes, presence of distant metastasis).

Cancer Testis (CT) genes are genes that are expressed in testis cells, but not expressed in somatic non pathologic cells. In cancers, CT genes are deregulated and are expressed ectopically in somatic cells. They appear as good candidate for cancer diagnosis.

Some works have intended to identify a “general” strategy for diagnosing lung cancer, by detecting cancer testis gene expression.

For instance, the international application WO 2009/121878 discloses the use of a minimal group of CT genes for identifying any somatic or ovarian cancer. However, even if specific genes, or combinations of genes, can be used for diagnosing cancer, there is no indication that these genes can be used to establish a reliable prognosis during a short or a long period.

The international application WO 2006/029176 discloses cancer testis genes (CT genes) as indicative markers for the cancer progression. A liste of 20 preferred genes is cited.

Therefore, there is a need to provide prognosis marker that can give specific evolution of tumoral progression, and patient survival, for instance by using CT genes.

Upon pathological diagnosis, the pathologist is able to define the histological subtype of lung cancer. Of all lung tumours, Small cell lung cancers (SCLC) represent the least frequent tumours with the worse outcome. Among Non Small Cell Lung cancers (NSCLC), the most frequent histological subtypes are squamous cell carcinoma (SQC), adenocarcinoma (ADK), large cell neuro-endocrine tumours (LCNE) and basaloid tumours (BAS), the former two being of better prognosis than the latter two. However, although the histological subtype of lung cancer represents one of the parameters influencing prognosis after surgery in operable lung cancer, patients with the same histological type of tumours have variable outcome, some die rapidly with signs of tumour aggressivity (local relapse after surgery, metastasis . . . ) whereas others survive longer and/or die of other causes. Hence tumour aggressivity is not specifically defined by its histological subtype, and there is a need for molecular markers defining particularly aggressive tumours within each given histological subtype, in order to adapt therapeutic approaches accordingly.

One aim of the invention is to provide a simple, rapid, easy-to-use and effective method for giving a prognosis of lung cancer.

Another aim of the invention is to provide a specific prognosis method for lung tumors that have been histologically classified.

The present invention relates to the use of at least one element chosen among:
at least 4 genes chosen among a group of 23 genes comprising or consisting of the nucleic acid sequences SEQ ID NO 1 to 23,
or fragments of said genes
or complementary sequences of said genes
or sequences having at least 80% homology with said genes or fragment thereof,
or protein coded by said genes, said proteins comprising or consisting of the amino acid sequences SEQ ID NO 24 to 46
or fragments of said proteins,
or antibodies directed against said proteins,
   said at least 4 genes being such that
a. at least one gene belongs to a first set A of 4 genes comprising or consisting of the nucleic acid sequences SEQ ID NO: 1 to 4,
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
   for the implementation of a prognosis method, preferably in vitro, of the survival rate of a patient afflicted by a lung cancer, said lung cancer being previously classified as a lung tumour belonging from the group consisting of adenocarcinma (ADK), Squamous cell carcinoma (SQC), Basaloid tumours (BAS) and Large Cell Neuroendocrine (LCNE),
   wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
   said prognosis being such that:
if none of the genes of said set A, B, C and D is expressed, the patient survival rate during a period of time from 30 to 120 months after the diagnosis of said lung cancer is from about 19% to about 88%, and
if at least one gene of one of said sets A, B, C and D is expressed, the patient survival rate during a period of time from 30 to 120 months after the diagnosis of said lung cancer is from about 0% to about 73%.

The present invention is based on the unexpected observation made by the Inventors that the expression of at least 4 genes of a group of 23 determined genes is sufficient to determine the survival rate of a patient afflicted by a lung cancer, said lung cancer being classified among the following histological tumors: (ADK adenocarcinoma), Squamous cell carcinoma (SQC), Basaloid tumours (BAS) and Large Cell Neuroendocrine (LCNE)

In other words, and as explained and exemplified hereafter, according to the invention the determination of the gene expression status on a ON/OFF basis of at least 4 genes chosen among a set of 23 genes allow to estimate at 30 months, or 60 months or 120 months after the diagnosis of a lung cancer, the probability of survival of an individual afflicted by one of the category of lung cancer belonging to the group of ADK, SQC, BAS and LCNE.

The prognosis method proposed by the Inventors can also be carried out by detecting the expression of proteins expressed by the above mentioned at least 4 genes chosen among proteins coded by said 23 genes, also on a absence/presence basis.

Another aspect of the invention is that the diagnosis can also be carried out by determining the presence, of at least 4 specific antibodies specifically recognising at least 4 proteins coded by said at least 4 genes mentioned above. The above antibodies are specific of one protein, each protein being coded by one gene of the set of 23 genes.

A key aspect of the invention is the concept of ON/OFF for gene expression and presence/absence for the encoded proteins and antibodies detecting these proteins. This specific approach has the advantage of simplifying the analyses and making them independent of complex statistical tests to measure variations in expression levels applied to the majority of the existing tests.

The ON/OFF status of gene expression is established by determination of a threshold of gene expression allowing them to decide on the ON/OFF status of a gene such that:
a. if a gene is expressed at a level lower than the threshold, the gene is considered as not expressed or weakly expressed (defined as OFF), and
b. if a gene is expressed at a level upper to the threshold, the gene is considered as being expressed (defined as ON).

The Inventors have identified 23 genes comprising or being constituted by the nucleic acid sequences SEQ ID NO 1 to 23, as being cancer testis genes (CT genes) that can be used to carry out the prognosis method according to the invention.

The above 23 genes have been identified as being liable to be “expressed” (form here by, expressed refers to the ON status and not expressed to the OFF status) in lung cancer cells, but not in healthy samples. In other words, the above 23 genes are such as
a. they are not expressed, or weakly expressed in lung cells, and
b. they maybe expressed in lung tumor cells.

By “not expressed” it is defined in the invention the fact that the transcription of a gene is either not carried out, or is not detectable by common techniques known in the art, such as Quantitative RT-PCR, Northern blot or when microarrays data are considered.

By “weakly expressed”, it is defined in the invention that a gene is expressed at a low level, meaning that the values are within the range of those measured for healthy tissue samples by Q-RT-PCR and by Northern blots or below the threshold when microarray data are considered. These values are considered as false-positive expressions, due to probe cross hybridization for instance. All the expression falling in these categories are considered as “OFF”

The difference between the absence of expression, or weak expression, and the expression determines its ON/OFF status, which is a key step of the invention.

Indeed, the Inventors have identified that the ON status of the above 23 genes is a key step to determine the prognosis of lung cancer.

On microarrays, the expression level of the above mentioned genes is determined by the fact that a threshold of expression has been identified by the Inventors allowing to determine expression (ON) and non-expression (OFF) of said genes. The threshold determination is detailed hereafter, in the Example section.

For the microarrays, the threshold enabling to determine the expression status of a gene (ON versus OFF) is calculated by using the signal mean value and distribution obtained from transcriptomic data (in the same technology) with the corresponding probes in a large number of somatic tissues (which do not express the genes).

A similar strategy enables determining a threshold for the presence/absence of the encoded proteins or antibodies. For each protein or antibody, the mean value and distribution of the signal intensities obtained in an appropriate number of control somatic tissues serves as a basis for calculating the threshold.

According to the invention, the prognosis is carried out as described hereafter:

By “expressed”, the invention defined that the transcript of a gene is detectable by the above known techniques while it is not detectable in healthy tissues or determined as being above the threshold when microarrays data are considered.

In the invention, the terms “for the implementation of a prognosis method” means “for carrying out a prognosis method”. The skilled person understand that he can obtain a prognosis according to the invention when using one of the element mentioned above. The 23 genes according to the invention have been classified by the Inventors in four sets:
a. a first set of 4 genes,
b. a second set of 3 genes
c. a third set of 6 genes, and
d. a fourth set of 9 genes.

The first set, also called in the invention set A, of 4 genes consists of the genes comprising or constituted by the nucleic acid sequences SEQ ID NO: 1, SEQ ID NO: 2, SEQ ID NO: 3, SEQ ID NO: 4.

The second set, also called in the invention set B, of 3 genes consists of the genes comprising or constituted by the nucleic acid sequences SEQ ID NO: 5, SEQ ID NO: 6 and SEQ ID NO: 7.

The third set, also called in the invention set C, of 6 genes consists of the genes comprising or constituted by the nucleic acid sequences SEQ ID NO: 8, SEQ ID NO: 9, SEQ ID NO: 10, SEQ ID NO: 11, SEQ ID NO: 12 and SEQ ID NO: 13.

The fourth set also called in the invention set D, of 9 genes consists of the genes comprising or constituted by the nucleic acid sequences SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23.

Set A is specific of BAS tumors, set B is specific of ADK tumors, set C is specific of SQC tumors and set D is specific of LCNE tumors.

The inventors have also defined subsets of each of the above sets A and B as follows:

Set A is divided into 4 subsets A1, A4 and A5, said subset being such that:
a. subset A1 consists of the genes comprising or being constituted by the nucleic acid sequences SEQ ID NO: 1 and SEQ ID NO: 2,
b. subset A4 consists of the gene comprising or being constituted by the nucleic acid sequence SEQ ID NO: 3, and
c. subset A5 consists of the genes comprising or being constituted by the nucleic acid sequence SEQ ID NO: 4,
   Set A can also be divided into the following subsets:
a. subset A1 consists of the genes comprising or being constituted by the nucleic acid sequences SEQ ID NO: 1 and SEQ ID NO: 2,
b. subset A2 consists of the genes comprising or being constituted by the nucleic acid sequences SEQ ID NO: 1, SEQ ID NO: 2 and SEQ ID NO: 3, and
c. subset A3 consists of the genes comprising or being constituted by the nucleic acid sequences SEQ ID NO: 1, SEQ ID NO: 2, SEQ ID NO: 3 and SEQ ID NO: 4.
   Set B is divided into 2 subsets B1 and B3, said subset being such that:
a. subset B1 consists of the genes comprising or being constituted by the nucleic acid sequences SEQ ID NO: 5 and SEQ ID NO: 6, and
b. subset B3 consists of the gene comprising or being constituted by the nucleic acid sequence SEQ ID NO: 7.
   Set B can also be divided into the following subsets:
c. subset B1 consists of the genes comprising or being constituted by the nucleic acid sequences SEQ ID NO: 5, and SEQ ID NO: 6,
d. subset B2 consists of the genes comprising or being constituted by the nucleic acid sequences SEQ ID NO: 5, SEQ ID NO: 6 and SEQ ID NO: 7.
   Set C is divided into 3 subsets C1, C4 and C5, said subset being such that:
a. subset C1 consists of the genes comprising or being constituted by the nucleic acid sequences SEQ ID NO: 8 and SEQ ID NO: 9,
b. subset C4 consists of the genes comprising or being constituted by the nucleic acid sequences SEQ ID NO: 10 and SEQ ID NO: 11, and
c. subset C5 consists of the genes comprising or being constituted by the nucleic acid sequences SEQ ID NO: 12 and SEQ ID NO: 13.
   Set C can also be divided into the following subsets:
a. subset C1 consists of the genes comprising or being constituted by the nucleic acid sequences SEQ ID NO: 8, and SEQ ID NO: 9,
b. subset C2 consists of the genes comprising or being constituted by the nucleic acid sequences SEQ ID NO: 8, SEQ ID NO: 9, SEQ ID NO: 10 and SEQ ID NO: 11, and
c. subset C3 consists of the genes comprising or being constituted by the nucleic acid sequences SEQ ID NO: 8, SEQ ID NO: 9, SEQ ID NO: 10, SEQ ID NO: 11, SEQ ID NO: 12 and SEQ ID NO: 13.
   Set D is divided into 3 subsets D1, D5, D6 and D7, said subset being such that:
a. subset D1 consists of the genes comprising or being constituted by the nucleic acid sequences SEQ ID NO: 14 and SEQ ID NO: 15,
b. subset D5 consists of the genes comprising or being constituted by the nucleic acid sequences SEQ ID NO: 16, SEQ ID NO: 17, SEQ ID NO: 18, SEQ ID NO: 19 and SEQ ID NO: 20,
c. subset D6 consists of the gene comprising or being constituted by the nucleic acid sequence SEQ ID NO: 21 and
d. subset D7 consists of the genes comprising or being constituted by the nucleic acid sequences SEQ ID NO: 22 and SEQ ID NO: 23.
   Set D can also be divided into the following subsets:
a. subset D1 consists of the genes comprising or being constituted by the nucleic acid sequences SEQ ID NO: 14 and SEQ ID NO: 15,
b. subset D2 consists of the genes comprising or being constituted by the nucleic acid sequences SEQ ID NO: 14 and SEQ ID NO: 15, SEQ ID NO: 16, SEQ ID NO: 17, SEQ ID NO: 18, SEQ ID NO: 19 and SEQ ID NO: 20,
c. subset D3 consists of the genes comprising or being constituted by the nucleic acid sequences SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21, and
d. subset D4 consists of the genes comprising or being constituted by the nucleic acid sequences SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.

Thus, the prognosis method according to the invention is such that, when determining the expression status of 4 genes on a ON/OFF basis, belonging to the set of 23 genes comprising or consisting of nucleic acid sequences SEQ ID NO: 1 to 23,
a. if none of said at least 4 genes are expressed, the patient survival rate at 30 months, 60 months or 120 months following the diagnosis is from about 19% to about 88% or more, and
b. if at least one gene of the sets A, B, C or D is expressed, according to the defined expression threshold, the patient survival rate at 30 months, 60 months or 120 months following the diagnosis is from about 0% to about 73%.

Therefore, the prognosis method according to the invention can be carried out as by measuring at least the expression of the following 720 combinations of genes: SEQ ID NO: 1+5+8+14, SEQ ID NO: 1+5+9+14, SEQ ID NO: 1+5+10+14, SEQ ID NO: 1+5+11+14, SEQ ID NO: 1+5+12+14, SEQ ID NO: 1+5+12+14, SEQ ID NO: 1+5+8+15, SEQ ID NO: 1+5+9+15, SEQ ID NO: 1+5+10+15, SEQ ID NO: 1+5+11+15, SEQ ID NO: 1+5+12+15, SEQ ID NO: 1+5+12+15, SEQ ID NO: 1+5+8+16, SEQ ID NO: 1+5+9+16, SEQ ID NO: 1+5+10+16, SEQ ID NO: 1+5+11+16, SEQ ID NO: 1+5+12+16, SEQ ID NO: 1+5+12+16, SEQ ID NO: 1+5+8+17, SEQ ID NO: 1+5+9+17, SEQ ID NO: 1+5+10+17, SEQ ID NO: 1+5+11+17, SEQ ID NO: 1+5+12+17, SEQ ID NO: 1+5+12+17, SEQ ID NO: 1+5+8+18, SEQ ID NO: 1+5+9+18, SEQ ID NO: 1+5+10+18, SEQ ID NO: 1+5+11+18, SEQ ID NO: 1+5+12+18, SEQ ID NO: 1+5+12+18, SEQ ID NO: 1+5+8+19, SEQ ID NO: 1+5+9+19, SEQ ID NO: 1+5+10+19, SEQ ID NO: 1+5+11+19, SEQ ID NO: 1+5+12+19, SEQ ID NO: 1+5+12+19, SEQ ID NO: 1+5+8+20, SEQ ID NO: 1+5+9+20, SEQ ID NO: 1+5+10+20, SEQ ID NO: 1+5+11+20, SEQ ID NO: 1+5+12+20, SEQ ID NO: 1+5+12+20, SEQ ID NO: 1+5+8+21, SEQ ID NO: 1+5+9+21, SEQ ID NO: 1+5+10+21, SEQ ID NO: 1+5+11+21, SEQ ID NO: 1+5+12+21, SEQ ID NO: 1+5+12+21, SEQ ID NO: 1+5+8+22, SEQ ID NO: 1+5+9+22, SEQ ID NO: 1+5+10+22, SEQ ID NO: 1+5+11+22, SEQ ID NO: 1+5+12+22, SEQ ID NO: 1+5+12+22, SEQ ID NO: 1+5+8+23, SEQ ID NO: 1+5+9+23, SEQ ID NO: 1+5+10+23, SEQ ID NO: 1+5+11+23, SEQ ID NO: 1+5+12+23, SEQ ID NO: 1+5+12+23, SEQ ID NO: 1+6+8+14, SEQ ID NO: 1+6+9+14, SEQ ID NO: 1+6+10+14, SEQ ID NO: 1+6+11+14, SEQ ID NO: 1+6+12+14, SEQ ID NO: 1+6+12+14, SEQ ID NO: 1+6+8+15, SEQ ID NO: 1+6+9+15, SEQ ID NO: 1+6+10+15, SEQ ID NO: 1+6+11+15, SEQ ID NO: 1+6+12+15, SEQ ID NO: 1+6+12+15, SEQ ID NO: 1+6+8+16, SEQ ID NO: 1+6+9+16, SEQ ID NO: 1+6+10+16, SEQ ID NO: 1+6+11+16, SEQ ID NO: 1+6+12+16, SEQ ID NO: 1+6+12+16, SEQ ID NO: 1+6+8+17, SEQ ID NO: 1+6+9+17, SEQ ID NO: 1+6+10+17, SEQ ID NO: 1+6+11+17, SEQ ID NO: 1+6+12+17, SEQ ID NO: 1+6+12+17, SEQ ID NO: 1+6+8+18, SEQ ID NO: 1+6+9+18, SEQ ID NO: 1+6+10+18, SEQ ID NO: 1+6+11+18, SEQ ID NO: 1+6+12+18, SEQ ID NO: 1+6+12+18, SEQ ID NO: 1+6+8+19, SEQ ID NO: 1+6+9+19, SEQ ID NO: 1+6+10+19, SEQ ID NO: 1+6+11+19, SEQ ID NO: 1+6+12+19, SEQ ID NO: 1+6+12+19, SEQ ID NO: 1+6+8+20, SEQ ID NO: 1+6+9+20, SEQ ID NO: 1+6+10+20, SEQ ID NO: 1+6+11+20, SEQ ID NO: 1+6+12+20, SEQ ID NO: 1+6+12+20, SEQ ID NO: 1+6+8+21, SEQ ID NO: 1+6+9+21, SEQ ID NO: 1+6+10+21, SEQ ID NO: 1+6+11+21, SEQ ID NO: 1+6+12+21, SEQ ID NO: 1+6+12+21, SEQ ID NO: 1+6+8+22, SEQ ID NO: 1+6+9+22, SEQ ID NO: 1+6+10+22, SEQ ID NO: 1+6+11+22, SEQ ID NO: 1+6+12+22, SEQ ID NO: 1+6+12+22, SEQ ID NO: 1+6+8+23, SEQ ID NO: 1+6+9+23, SEQ ID NO: 1+6+10+23, SEQ ID NO: 1+6+11+23, SEQ ID NO: 1+6+12+23, SEQ ID NO: 1+6+12+23, SEQ ID NO: 1+7+8+14, SEQ ID NO: 1+7+9+14, SEQ ID NO: 1+7+10+14, SEQ ID NO: 1+7+11+14, SEQ ID NO: 1+7+12+14, SEQ ID NO: 1+7+12+14, SEQ ID NO: 1+7+8+15, SEQ ID NO: 1+7+9+15, SEQ ID NO: 1+7+10+15, SEQ ID NO: 1+7+11+15, SEQ ID NO: 1+7+12+15, SEQ ID NO: 1+7+12+15, SEQ ID NO: 1+7+8+16, SEQ ID NO: 1+7+9+16, SEQ ID NO: 1+7+10+16, SEQ ID NO: 1+7+11+16, SEQ ID NO: 1+7+12+16, SEQ ID NO: 1+7+12+16, SEQ ID NO: 1+7+8+17, SEQ ID NO: 1+7+9+17, SEQ ID NO: 1+7+10+17, SEQ ID NO: 1+7+11+17, SEQ ID NO: 1+7+12+17, SEQ ID NO: 1+7+12+17, SEQ ID NO: 1+7+8+18, SEQ ID NO: 1+7+9+18, SEQ ID NO: 1+7+10+18, SEQ ID NO: 1+7+11+18, SEQ ID NO: 1+7+12+18, SEQ ID NO: 1+7+12+18, SEQ ID NO: 1+7+8+19, SEQ ID NO: 1+7+9+19, SEQ ID NO: 1+7+10+19, SEQ ID NO: 1+7+11+19, SEQ ID NO: 1+7+12+19, SEQ ID NO: 1+7+12+19, SEQ ID NO: 1+7+8+20, SEQ ID NO: 1+7+9+20, SEQ ID NO: 1+7+10+20, SEQ ID NO: 1+7+11+20, SEQ ID NO: 1+7+12+20, SEQ ID NO: 1+7+12+20, SEQ ID NO: 1+7+8+21, SEQ ID NO: 1+7+9+21, SEQ ID NO: 1+7+10+21, SEQ ID NO: 1+7+11+21, SEQ ID NO: 1+7+12+21, SEQ ID NO: 1+7+12+21, SEQ ID NO: 1+7+8+22, SEQ ID NO: 1+7+9+22, SEQ ID NO: 1+7+10+22, SEQ ID NO: 1+7+11+22, SEQ ID NO: 1+7+12+22, SEQ ID NO: 1+7+12+22, SEQ ID NO: 1+7+8+23, SEQ ID NO: 1+7+9+23, SEQ ID NO: 1+7+10+23, SEQ ID NO: 1+7+11+23, SEQ ID NO: 1+7+12+23, SEQ ID NO: 1+7+12+23, SEQ ID NO: 2+5+8+14, SEQ ID NO: 2+5+9+14, SEQ ID NO: 1+5+10+14, SEQ ID NO: 2+5+11+14, SEQ ID NO: 2+5+12+14, SEQ ID NO: 2+5+12+14, SEQ ID NO: 2+5+8+15, SEQ ID NO: 2+5+9+15, SEQ ID NO: 1+5+10+15, SEQ ID NO: 2+5+11+15, SEQ ID NO: 2+5+12+15, SEQ ID NO: 2+5+12+15, SEQ ID NO: 2+5+8+16, SEQ ID NO: 2+5+9+16, SEQ ID NO: 1+5+10+16, SEQ ID NO: 2+5+11+16, SEQ ID NO: 2+5+12+16, SEQ ID NO: 2+5+12+16, SEQ ID NO: 2+5+8+17, SEQ ID NO: 2+5+9+17, SEQ ID NO: 1+5+10+17, SEQ ID NO: 2+5+11+17, SEQ ID NO: 2+5+12+17, SEQ ID NO: 2+5+12+17, SEQ ID NO: 2+5+8+18, SEQ ID NO: 2+5+9+18, SEQ ID NO: 1+5+10+18, SEQ ID NO: 2+5+11+18, SEQ ID NO: 2+5+12+18, SEQ ID NO: 2+5+12+18, SEQ ID NO: 2+5+8+19, SEQ ID NO: 2+5+9+19, SEQ ID NO: 1+5+10+19, SEQ ID NO: 2+5+11+19, SEQ ID NO: 2+5+12+19, SEQ ID NO: 2+5+12+19, SEQ ID NO: 2+5+8+20, SEQ ID NO: 2+5+9+20, SEQ ID NO: 1+5+10+20, SEQ ID NO: 2+5+11+20, SEQ ID NO: 2+5+12+20, SEQ ID NO: 2+5+12+20, SEQ ID NO: 2+5+8+21, SEQ ID NO: 2+5+9+21, SEQ ID NO: 1+5+10+21, SEQ ID NO: 2+5+11+21, SEQ ID NO: 2+5+12+21, SEQ ID NO: 2+5+12+21, SEQ ID NO: 2+5+8+22, SEQ ID NO: 2+5+9+22, SEQ ID NO: 1+5+10+22, SEQ ID NO: 2+5+11+22, SEQ ID NO: 2+5+12+22, SEQ ID NO: 2+5+12+22, SEQ ID NO: 2+5+8+23, SEQ ID NO: 2+5+9+23, SEQ ID NO: 1+5+10+23, SEQ ID NO: 2+5+11+23, SEQ ID NO: 2+5+12+23, SEQ ID NO: 2+5+12+23, SEQ ID NO: 2+6+8+14, SEQ ID NO: 2+6+9+14, SEQ ID NO: 2+6+10+14, SEQ ID NO: 2+6+11+14, SEQ ID NO: 2+6+12+14, SEQ ID NO: 2+6+12+14, SEQ ID NO: 2+6+8+15, SEQ ID NO: 2+6+9+15, SEQ ID NO: 2+6+10+15, SEQ ID NO: 2+6+11+15, SEQ ID NO: 2+6+12+15, SEQ ID NO: 2+6+12+15, SEQ ID NO: 2+6+8+16, SEQ ID NO: 2+6+9+16, SEQ ID NO: 2+6+10+16, SEQ ID NO: 2+6+11+16, SEQ ID NO: 2+6+12+16, SEQ ID NO: 2+6+12+16, SEQ ID NO: 2+6+8+17, SEQ ID NO: 2+6+9+17, SEQ ID NO: 2+6+10+17, SEQ ID NO: 2+6+11+17, SEQ ID NO: 2+6+12+17, SEQ ID NO: 2+6+12+17, SEQ ID NO: 2+6+8+18, SEQ ID NO: 2+6+9+18, SEQ ID NO: 2+6+10+18, SEQ ID NO: 2+6+11+18, SEQ ID NO: 2+6+12+18, SEQ ID NO: 2+6+12+18, SEQ ID NO: 2+6+8+19, SEQ ID NO: 2+6+9+19, SEQ ID NO: 2+6+10+19, SEQ ID NO: 2+6+11+19, SEQ ID NO: 2+6+12+19, SEQ ID NO: 2+6+12+19, SEQ ID NO: 2+6+8+20, SEQ ID NO: 2+6+9+20, SEQ ID NO: 2+6+10+20, SEQ ID NO: 2+6+11+20, SEQ ID NO: 2+6+12+20, SEQ ID NO: 2+6+12+20, SEQ ID NO: 2+6+8+21, SEQ ID NO: 2+6+9+21, SEQ ID NO: 2+6+10+21, SEQ ID NO: 2+6+11+21, SEQ ID NO: 2+6+12+21, SEQ ID NO: 2+6+12+21, SEQ ID NO: 2+6+8+22, SEQ ID NO: 2+6+9+22, SEQ ID NO: 2+6+10+22, SEQ ID NO: 2+6+11+22, SEQ ID NO: 2+6+12+22, SEQ ID NO: 2+6+12+22, SEQ ID NO: 2+6+8+23, SEQ ID NO: 2+6+9+23, SEQ ID NO: 2+6+10+23, SEQ ID NO: 2+6+11+23, SEQ ID NO: 2+6+12+23, SEQ ID NO: 2+6+12+23, SEQ ID NO: 2+7+8+14, SEQ ID NO: 2+7+9+14, SEQ ID NO: 2+7+10+14, SEQ ID NO: 2+7+11+14, SEQ ID NO: 2+7+12+14, SEQ ID NO: 2+7+12+14, SEQ ID NO: 2+7+8+15, SEQ ID NO: 2+7+9+15, SEQ ID NO: 2+7+10+15, SEQ ID NO: 2+7+11+15, SEQ ID NO: 2+7+12+15, SEQ ID NO: 2+7+12+15, SEQ ID NO: 2+7+8+16, SEQ ID NO: 2+7+9+16, SEQ ID NO: 2+7+10+16, SEQ ID NO: 2+7+11+16, SEQ ID NO: 2+7+12+16, SEQ ID NO: 2+7+12+16, SEQ ID NO: 2+7+8+17, SEQ ID NO: 2+7+9+17, SEQ ID NO: 2+7+10+17, SEQ ID NO: 2+7+11+17, SEQ ID NO: 2+7+12+17, SEQ ID NO: 2+7+12+17, SEQ ID NO: 2+7+8+18, SEQ ID NO: 2+7+9+18, SEQ ID NO: 2+7+10+18, SEQ ID NO: 2+7+11+18, SEQ ID NO: 2+7+12+18, SEQ ID NO: 2+7+12+18, SEQ ID NO: 2+7+8+19, SEQ ID NO: 2+7+9+19, SEQ ID NO: 2+7+10+19, SEQ ID NO: 2+7+11+19, SEQ ID NO: 2+7+12+19, SEQ ID NO: 2+7+12+19, SEQ ID NO: 2+7+8+20, SEQ ID NO: 2+7+9+20, SEQ ID NO: 2+7+10+20, SEQ ID NO: 2+7+11+20, SEQ ID NO: 2+7+12+20, SEQ ID NO: 2+7+12+20, SEQ ID NO: 2+7+8+21, SEQ ID NO: 2+7+9+21, SEQ ID NO: 2+7+10+21, SEQ ID NO: 2+7+11+21, SEQ ID NO: 2+7+12+21, SEQ ID NO: 2+7+12+21, SEQ ID NO: 2+7+8+22, SEQ ID NO: 2+7+9+22, SEQ ID NO: 2+7+10+22, SEQ ID NO: 2+7+11+22, SEQ ID NO: 2+7+12+22, SEQ ID NO: 2+7+12+22, SEQ ID NO: 2+7+8+23, SEQ ID NO: 2+7+9+23, SEQ ID NO: 2+7+10+23, SEQ ID NO: 2+7+11+23, SEQ ID NO: 2+7+12+23, SEQ ID NO: 2+7+12+23, SEQ ID NO: 3+5+8+14, SEQ ID NO: 3+5+9+14, SEQ ID NO: 3+5+10+14, SEQ ID NO: 3+5+11+14, SEQ ID NO: 3+5+12+14, SEQ ID NO: 3+5+12+14, SEQ ID NO: 3+5+8+15, SEQ ID NO: 3+5+9+15, SEQ ID NO: 3+5+10+15, SEQ ID NO: 3+5+11+15, SEQ ID NO: 3+5+12+15, SEQ ID NO: 3+5+12+15, SEQ ID NO: 3+5+8+16, SEQ ID NO: 3+5+9+16, SEQ ID NO: 3+5+10+16, SEQ ID NO: 3+5+11+16, SEQ ID NO: 3+5+12+16, SEQ ID NO: 3+5+12+16, SEQ ID NO: 3+5+8+17, SEQ ID NO: 3+5+9+17, SEQ ID NO: 3+5+10+17, SEQ ID NO: 3+5+11+17, SEQ ID NO: 3+5+12+17, SEQ ID NO: 3+5+12+17, SEQ ID NO: 3+5+8+18, SEQ ID NO: 3+5+9+18, SEQ ID NO: 3+5+10+18, SEQ ID NO: 3+5+11+18, SEQ ID NO: 3+5+12+18, SEQ ID NO: 3+5+12+18, SEQ ID NO: 3+5+8+19, SEQ ID NO: 3+5+9+19, SEQ ID NO: 3+5+10+19, SEQ ID NO: 3+5+11+19, SEQ ID NO: 3+5+12+19, SEQ ID NO: 3+5+12+19, SEQ ID NO: 3+5+8+20, SEQ ID NO: 3+5+9+20, SEQ ID NO: 3+5+10+20, SEQ ID NO: 3+5+11+20, SEQ ID NO: 3+5+12+20, SEQ ID NO: 3+5+12+20, SEQ ID NO: 3+5+8+21, SEQ ID NO: 3+5+9+21, SEQ ID NO: 3+5+10+21, SEQ ID NO: 3+5+11+21, SEQ ID NO: 3+5+12+21, SEQ ID NO: 3+5+12+21, SEQ ID NO: 3+5+8+22, SEQ ID NO: 3+5+9+22, SEQ ID NO: 3+5+10+22, SEQ ID NO: 3+5+11+22, SEQ ID NO: 3+5+12+22, SEQ ID NO: 3+5+12+22, SEQ ID NO: 3+5+8+23, SEQ ID NO: 3+5+9+23, SEQ ID NO: 3+5+10+23, SEQ ID NO: 3+5+11+23, SEQ ID NO: 3+5+12+23, SEQ ID NO: 3+5+12+23, SEQ ID NO: 3+6+8+14, SEQ ID NO: 3+6+9+14, SEQ ID NO: 3+6+10+14, SEQ ID NO: 3+6+11+14, SEQ ID NO: 3+6+12+14, SEQ ID NO: 3+6+12+14, SEQ ID NO: 3+6+8+15, SEQ ID NO: 3+6+9+15, SEQ ID NO: 3+6+10+15, SEQ ID NO: 3+6+11+15, SEQ ID NO: 3+6+12+15, SEQ ID NO: 3+6+12+15, SEQ ID NO: 3+6+8+16, SEQ ID NO: 3+6+9+16, SEQ ID NO: 3+6+10+16, SEQ ID NO: 3+6+11+16, SEQ ID NO: 3+6+12+16, SEQ ID NO: 3+6+12+16, SEQ ID NO: 3+6+8+17, SEQ ID NO: 3+6+9+17, SEQ ID NO: 3+6+10+17, SEQ ID NO: 3+6+11+17, SEQ ID NO: 3+6+12+17, SEQ ID NO: 3+6+12+17, SEQ ID NO: 3+6+8+18, SEQ ID NO: 3+6+9+18, SEQ ID NO: 3+6+10+18, SEQ ID NO: 3+6+11+18, SEQ ID NO: 3+6+12+18, SEQ ID NO: 3+6+12+18, SEQ ID NO: 3+6+8+19, SEQ ID NO: 3+6+9+19, SEQ ID NO: 3+6+10+19, SEQ ID NO: 3+6+11+19, SEQ ID NO: 3+6+12+19, SEQ ID NO: 3+6+12+19, SEQ ID NO: 3+6+8+20, SEQ ID NO: 3+6+9+20, SEQ ID NO: 3+6+10+20, SEQ ID NO: 3+6+11+20, SEQ ID NO: 3+6+12+20, SEQ ID NO: 3+6+12+20, SEQ ID NO: 3+6+8+21, SEQ ID NO: 3+6+9+21, SEQ ID NO: 3+6+10+21, SEQ ID NO: 3+6+11+21, SEQ ID NO: 3+6+12+21, SEQ ID NO: 3+6+12+21, SEQ ID NO: 3+6+8+22, SEQ ID NO: 3+6+9+22, SEQ ID NO: 3+6+10+22, SEQ ID NO: 3+6+11+22, SEQ ID NO: 3+6+12+22, SEQ ID NO: 3+6+12+22, SEQ ID NO: 3+6+8+23, SEQ ID NO: 3+6+9+23, SEQ ID NO: 3+6+10+23, SEQ ID NO: 3+6+11+23, SEQ ID NO: 3+6+12+23, SEQ ID NO: 3+6+12+23, SEQ ID NO: 3+7+8+14, SEQ ID NO: 3+7+9+14, SEQ ID NO: 3+7+10+14, SEQ ID NO: 3+7+11+14, SEQ ID NO: 3+7+12+14, SEQ ID NO: 3+7+12+14, SEQ ID NO: 3+7+8+15, SEQ ID NO: 3+7+9+15, SEQ ID NO: 3+7+10+15, SEQ ID NO: 3+7+11+15, SEQ ID NO: 3+7+12+15, SEQ ID NO: 3+7+12+15, SEQ ID NO: 3+7+8+16, SEQ ID NO: 3+7+9+16, SEQ ID NO: 3+7+10+16, SEQ ID NO: 3+7+11+16, SEQ ID NO: 3+7+12+16, SEQ ID NO: 3+7+12+16, SEQ ID NO: 3+7+8+17, SEQ ID NO: 3+7+9+17, SEQ ID NO: 3+7+10+17, SEQ ID NO: 3+7+11+17, SEQ ID NO: 3+7+12+17, SEQ ID NO: 3+7+12+17, SEQ ID NO: 3+7+8+18, SEQ ID NO: 3+7+9+18, SEQ ID NO: 3+7+10+18, SEQ ID NO: 3+7+11+18, SEQ ID NO: 3+7+12+18, SEQ ID NO: 3+7+12+18, SEQ ID NO: 3+7+8+19, SEQ ID NO: 3+7+9+19, SEQ ID NO: 3+7+10+19, SEQ ID NO: 3+7+11+19, SEQ ID NO: 3+7+12+19, SEQ ID NO: 3+7+12+19, SEQ ID NO: 3+7+8+20, SEQ ID NO: 3+7+9+20, SEQ ID NO: 3+7+10+20, SEQ ID NO: 3+7+11+20, SEQ ID NO: 3+7+12+20, SEQ ID NO: 3+7+12+20, SEQ ID NO: 3+7+8+21, SEQ ID NO: 3+7+9+21, SEQ ID NO: 3+7+10+21, SEQ ID NO: 3+7+11+21, SEQ ID NO: 3+7+12+21, SEQ ID NO: 3+7+12+21, SEQ ID NO: 3+7+8+22, SEQ ID NO: 3+7+9+22, SEQ ID NO: 3+7+10+22, SEQ ID NO: 3+7+11+22, SEQ ID NO: 3+7+12+22, SEQ ID NO: 3+7+12+22, SEQ ID NO: 3+7+8+23, SEQ ID NO: 3+7+9+23, SEQ ID NO: 3+7+10+23, SEQ ID NO: 3+7+11+23, SEQ ID NO: 3+7+12+23, SEQ ID NO: 3+7+12+23, SEQ ID NO: 4+5+8+14, SEQ ID NO: 4+5+9+14, SEQ ID NO: 4+5+10+14, SEQ ID NO: 4+5+11+14, SEQ ID NO: 4+5+12+14, SEQ ID NO: 4+5+12+14, SEQ ID NO: 4+5+8+15, SEQ ID NO: 4+5+9+15, SEQ ID NO: 4+5+10+15, SEQ ID NO: 4+5+11+15, SEQ ID NO: 4+5+12+15, SEQ ID NO: 4+5+12+15, SEQ ID NO: 4+5+8+16, SEQ ID NO: 4+5+9+16, SEQ ID NO: 4+5+10+16, SEQ ID NO: 4+5+11+16, SEQ ID NO: 4+5+12+16, SEQ ID NO: 4+5+12+16, SEQ ID NO: 4+5+8+17, SEQ ID NO: 4+5+9+17, SEQ ID NO: 4+5+10+17, SEQ ID NO: 4+5+11+17, SEQ ID NO: 4+5+12+17, SEQ ID NO: 4+5+12+17, SEQ ID NO: 4+5+8+18, SEQ ID NO: 4+5+9+18, SEQ ID NO: 4+5+10+18, SEQ ID NO: 4+5+11+18, SEQ ID NO: 4+5+12+18, SEQ ID NO: 4+5+12+18, SEQ ID NO: 4+5+8+19, SEQ ID NO: 4+5+9+19, SEQ ID NO: 4+5+10+19, SEQ ID NO: 4+5+11+19, SEQ ID NO: 4+5+12+19, SEQ ID NO: 4+5+12+19, SEQ ID NO: 4+5+8+20, SEQ ID NO: 4+5+9+20, SEQ ID NO: 4+5+10+20, SEQ ID NO: 4+5+11+20, SEQ ID NO: 4+5+12+20, SEQ ID NO: 4+5+12+20, SEQ ID NO: 4+5+8+21, SEQ ID NO: 4+5+9+21, SEQ ID NO: 4+5+10+21, SEQ ID NO: 4+5+11+21, SEQ ID NO: 4+5+12+21, SEQ ID NO: 4+5+12+21, SEQ ID NO: 4+5+8+22, SEQ ID NO: 4+5+9+22, SEQ ID NO: 4+5+10+22, SEQ ID NO: 4+5+11+22, SEQ ID NO: 4+5+12+22, SEQ ID NO: 4+5+12+22, SEQ ID NO: 4+5+8+23, SEQ ID NO: 4+5+9+23, SEQ ID NO: 4+5+10+23, SEQ ID NO: 4+5+11+23, SEQ ID NO: 4+5+12+23, SEQ ID NO: 4+5+12+23, SEQ ID NO: 4+6+8+14, SEQ ID NO: 4+6+9+14, SEQ ID NO: 4+6+10+14, SEQ ID NO: 4+6+11+14, SEQ ID NO: 4+6+12+14, SEQ ID NO: 4+6+12+14, SEQ ID NO: 4+6+8+15, SEQ ID NO: 4+6+9+15, SEQ ID NO: 4+6+10+15, SEQ ID NO: 4+6+11+15, SEQ ID NO: 4+6+12+15, SEQ ID NO: 4+6+12+15, SEQ ID NO: 4+6+8+16, SEQ ID NO: 4+6+9+16, SEQ ID NO: 4+6+10+16, SEQ ID NO: 4+6+11+16, SEQ ID NO: 4+6+12+16, SEQ ID NO: 4+6+12+16, SEQ ID NO: 4+6+8+17, SEQ ID NO: 4+6+9+17, SEQ ID NO: 4+6+10+17, SEQ ID NO: 4+6+11+17, SEQ ID NO: 4+6+12+17, SEQ ID NO: 4+6+12+17, SEQ ID NO: 4+6+8+18, SEQ ID NO: 4+6+9+18, SEQ ID NO: 4+6+10+18, SEQ ID NO: 4+6+11+18, SEQ ID NO: 4+6+12+18, SEQ ID NO: 4+6+12+18, SEQ ID NO: 4+6+8+19, SEQ ID NO: 4+6+9+19, SEQ ID NO: 4+6+10+19, SEQ ID NO: 4+6+11+19, SEQ ID NO: 4+6+12+19, SEQ ID NO: 4+6+12+19, SEQ ID NO: 4+6+8+20, SEQ ID NO: 4+6+9+20, SEQ ID NO: 4+6+10+20, SEQ ID NO: 4+6+11+20, SEQ ID NO: 4+6+12+20, SEQ ID NO: 4+6+12+20, SEQ ID NO: 4+6+8+21, SEQ ID NO: 4+6+9+21, SEQ ID NO: 4+6+10+21, SEQ ID NO: 4+6+11+21, SEQ ID NO: 4+6+12+21, SEQ ID NO: 4+6+12+21, SEQ ID NO: 4+6+8+22, SEQ ID NO: 4+6+9+22, SEQ ID NO: 4+6+10+22, SEQ ID NO: 4+6+11+22, SEQ ID NO: 4+6+12+22, SEQ ID NO: 4+6+12+22, SEQ ID NO: 4+6+8+23, SEQ ID NO: 4+6+9+23, SEQ ID NO: 4+6+10+23, SEQ ID NO: 4+6+11+23, SEQ ID NO: 4+6+12+23, SEQ ID NO: 4+6+12+23, SEQ ID NO: 4+7+8+14, SEQ ID NO: 4+7+9+14, SEQ ID NO: 4+7+10+14, SEQ ID NO: 4+7+11+14, SEQ ID NO: 4+7+12+14, SEQ ID NO: 4+7+12+14, SEQ ID NO: 4+7+8+15, SEQ ID NO: 4+7+9+15, SEQ ID NO: 4+7+10+15, SEQ ID NO: 4+7+11+15, SEQ ID NO: 4+7+12+15, SEQ ID NO: 4+7+12+15, SEQ ID NO: 4+7+8+16, SEQ ID NO: 4+7+9+16, SEQ ID NO: 4+7+10+16, SEQ ID NO: 4+7+11+16, SEQ ID NO: 4+7+12+16, SEQ ID NO: 4+7+12+16, SEQ ID NO: 4+7+8+17, SEQ ID NO: 4+7+9+17, SEQ ID NO: 4+7+10+17, SEQ ID NO: 4+7+11+17, SEQ ID NO: 4+7+12+17, SEQ ID NO: 4+7+12+17, SEQ ID NO: 4+7+8+18, SEQ ID NO: 4+7+9+18, SEQ ID NO: 4+7+10+18, SEQ ID NO: 4+7+11+18, SEQ ID NO: 4+7+12+18, SEQ ID NO: 4+7+12+18, SEQ ID NO: 4+7+8+19, SEQ ID NO: 4+7+9+19, SEQ ID NO: 4+7+10+19, SEQ ID NO: 4+7+11+19, SEQ ID NO: 4+7+12+19, SEQ ID NO: 4+7+12+19, SEQ ID NO: 4+7+8+20, SEQ ID NO: 4+7+9+20, SEQ ID NO: 4+7+10+20, SEQ ID NO: 4+7+11+20, SEQ ID NO: 4+7+12+20, SEQ ID NO: 4+7+12+20, SEQ ID NO: 4+7+8+21, SEQ ID NO: 4+7+9+21, SEQ ID NO: 4+7+10+21, SEQ ID NO: 4+7+11+21, SEQ ID NO: 4+7+12+21, SEQ ID NO: 4+7+12+21, SEQ ID NO: 4+7+8+22, SEQ ID NO: 4+7+9+22, SEQ ID NO: 4+7+10+22, SEQ ID NO: 4+7+11+22, SEQ ID NO: 4+7+12+22, SEQ ID NO: 4+7+12+22, SEQ ID NO: 4+7+8+23, SEQ ID NO: 4+7+9+23, SEQ ID NO: 4+7+10+23, SEQ ID NO: 4+7+11+23, SEQ ID NO: 4+7+12+23 and SEQ ID NO: 4+7+12+23.

For instance, the prognosis method according to the invention can be carried out by determining the expression status of the above combinations SEQ ID NO: 1+SEQ ID NO: 5+SEQ ID NO: 13+SEQ ID NO: 23, wherein
a. if neither SEQ ID NO: 1 nor SEQ ID NO: 5, nor SEQ ID NO: 13 nor SEQ ID NO: 23 is expressed, the patient survival rate at 30 months, 60 months or 120 months following the diagnosis is from about 19% to about 88% or more, and
b. if either SEQ ID NO: 1 or SEQ ID NO: 5, or SEQ ID NO: 13 or SEQ ID NO: 23 is expressed, the patient survival rate at 30 months, 60 months or 120 months following the diagnosis is from about 0% to about 73%.

According to the invention, the terms “about X %” means that the percentage of survival proposed for the prognosis method have to be considered with a standard deviation corresponding to individual variability. This standard deviation is 5%

To summarise, the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first set A of 4 genes comprising or consisting of the nucleic acid sequences SEQ ID NO: 1 to 4,
   b. at least one gene belongs to a second set B of 3 genes comprising or consisting of the nucleic acid sequences SEQ ID NO: 5 to 7
   c. at least one gene belongs to a third set C of 6 genes comprising or consisting of the nucleic acid sequences SEQ ID NO: 8 to 13, and
   d. at least one gene belongs to a fourth set D of 9 genes comprising or consisting of the nucleic acid sequences SEQ ID NO: 14-23,
at least 4 proteins being such that
   a. at least one protein belongs to a first set AP of 4 proteins comprising or consisting of the amino acid sequences SEQ ID NO: 24-27,
   b. at least one protein belongs to a second set BP of 3 proteins comprising or consisting of the amino acid sequences SEQ ID NO: 28-30, and
   c. at least one protein belongs to a third set CP of 6 proteins comprising or consisting of the amino acid sequences SEQ ID NO: 31-36,
   d. at least one protein belongs to a fourth set DP of 9 proteins comprising or consisting of the amino acid sequences SEQ ID NO: 37-46,
at least 4 antibodies directed against said 4 proteins being such that
   a. at least one antibody that ineract with at least one protein that belongs to a first set AP of 4 proteins comprising or consisting of the amino acid sequences SEQ ID NO: 24-27,
   b. at least one antibody that ineract with at least one protein that belongs a second set BP of 3 proteins comprising or consisting of the amino acid sequences SEQ ID NO: 28-30,
   c. at least one antibody that ineract with at least one protein that belongs a third set CP of 6 proteins comprising or consisting of the amino acid sequences SEQ ID NO: 31-36, and
   d. at least one antibody that ineract with at least one protein that belongs a fourth set DP of 9 proteins comprising or consisting of the amino acid sequences SEQ ID NO: 37-46,
      for the implementation of a prognosis method, preferably in vitro, of the survival rate of a patient afflicted by a lung cancer, said lung cancer being previously classified as a lung tumour belonging from the group consisting of ADK, SQC, BAS et LCNE,
      wherein
the nucleic acid sequences SEQ ID NO: 1 to 4 and/or the amino acid sequences SEQ ID NO: 24 to 27 are expressed in BAS,
the nucleic acid sequences SEQ ID NO: 5 to 7 and/or the amino acid sequences SEQ ID NO: 28 to 30 are expressed in ADK,
the nucleic acid sequences SEQ ID NO: 8 to 13 and/or the amino acid sequences SEQ ID NO: 31 to 36 are expressed in SQC,
the nucleic acid sequences SEQ ID NO: 14 to 23 and/or the amino acid sequences SEQ ID NO: 37 to 46 are expressed in LCNE,
   said prognosis being such that:
   either
if none of the 23 genes of said set is expressed, the patient survival rate during a period of time from 30 to 120 months after the diagnosis of said lung cancer is from about 59% to about 78% or more, and
if at least one gene of at least one set A, B, C or D is expressed, the patient survival rate during a period of time from 30 to 120 months after the diagnosis of said lung cancer is about from about 3% to about 70%,
   or
if none of the 23 proteins of said set is expressed, the patient survival rate during a period of time from 30 to 120 months after the diagnosis of said lung cancer is from about 59% to about 78% or more, and
if at least one protein of at least one set AP, BP, CP or DP is expressed, the patient survival rate during a period of time from 30 to 120 months after the diagnosis of said lung cancer is about from about 3% to about 70%,
   or
if none of the antibodies directed against said 23 proteins of said set is expressed, the patient survival rate during a period of time from 30 to 120 months after the diagnosis of said lung cancer is from about 59% to about 78% or more, and
if at least one antibody directed against one protein of at least one set AP, BP, CP or DP is expressed, the patient survival rate during a period of time from 30 to 120 months after the diagnosis of said lung cancer is about from about 3% to about 70%,
   wherein said gene, protein or antibody is determined as being expressed when:
either it is expressed in a sample of a patient afflicted by a lung cancer but not in a control sample of an healthy individual,
or it is expressed at a level above a threshold corresponding to a background signal observed in a series of reference healthy tissues for each detection method, and
   said gene, protein or antibody is determined as being not expressed when:
it is neither expressed in a sample of a patient afflicted by a lung cancer nor in a control sample of an healthy individual,
or it is expressed in a sample of a patient afflicted by a lung cancer at a level substantially equal or inferior to the level in a control sample of an healthy individual.

According to the invention, “a control sample of an healthy individual” corresponds to a somatic tissue in which the CT gene is not expressed or weakly expressed as defined above.

In the invention “is expressed at a level above a threshold corresponding to a background signal observed in a series of reference healthy tissues for each detection method” means that the threshold, which corresponds to the key step of the invention, is determined by measuring the background signal in negative control samples of healthy tissues, in which there is no expression of CT genes.

This background signal depends upon the method used to carry out the invention. However, it is easy for a skilled person to measure such threshold whatever the method used, i.e.:
if the number of control samples is significantly statistically representative, e.g. at least 30 independent control samples, and the background signal of these control sample follows a normal distribution (Gaussian distribution), the threshold is determined by the mean+2 standard deviations of the background signal measured in the control samples,
If the number of control sample is not statically representative, e.g. less than 30 independent control samples, and the background signal of these control sample does not follow a normal distribution, the threshold is determined as being the maximal value of the background signal measured in the control samples.

According to the invention, the gene comprising or consisting of the nucleic acid sequence SEQ ID NO: 1 codes for the protein comprising or consisting of the amino acid sequence SEQ ID NO: 24, the gene comprising or consisting of the nucleic acid sequence SEQ ID NO: 2 codes for the protein comprising or consisting of the amino acid sequence SEQ ID NO: 25, the gene comprising or consisting of the nucleic acid sequence SEQ ID NO: 3 codes for the protein comprising or consisting of the amino acid sequence SEQ ID NO: 26, the gene comprising or consisting of the nucleic acid sequence SEQ ID NO: 4 codes for the protein comprising or consisting of the amino acid sequence SEQ ID NO: 27, the gene comprising or consisting of the nucleic acid sequence SEQ ID NO: 5 codes for the protein comprising or consisting of the amino acid sequence SEQ ID NO: 28, the gene comprising or consisting of the nucleic acid sequence SEQ ID NO: 6 codes for the protein comprising or consisting of the amino acid sequence SEQ ID NO: 29, the gene comprising or consisting of the nucleic acid sequence SEQ ID NO: 7 codes for the protein comprising or consisting of the amino acid sequence SEQ ID NO: 30, the gene comprising or consisting of the nucleic acid sequence SEQ ID NO: 8 codes for the protein comprising or consisting of the amino acid sequence SEQ ID NO: 31, the gene comprising or consisting of the nucleic acid sequence SEQ ID NO: 9 codes for the protein comprising or consisting of the amino acid sequence SEQ ID NO: 32, the gene comprising or consisting of the nucleic acid sequence SEQ ID NO: 10 codes for the protein comprising or consisting of the amino acid sequence SEQ ID NO: 33, the gene comprising or consisting of the nucleic acid sequence SEQ ID NO: 11 codes for the protein comprising or consisting of the amino acid sequence SEQ ID NO: 34, the gene comprising or consisting of the nucleic acid sequence SEQ ID NO: 12 codes for the protein comprising or consisting of the amino acid sequence SEQ ID NO: 35, the gene comprising or consisting of the nucleic acid sequence SEQ ID NO: 13 codes for the protein comprising or consisting of the amino acid sequence SEQ ID NO: 36, the gene comprising or consisting of the nucleic acid sequence SEQ ID NO: 14 codes for the protein comprising or consisting of the amino acid sequence SEQ ID NO: 37, the gene comprising or consisting of the nucleic acid sequence SEQ ID NO: 15 codes for the protein comprising or consisting of the amino acid sequence SEQ ID NO: 38, the gene comprising or consisting of the nucleic acid sequence SEQ ID NO: 16 codes for the protein comprising or consisting of the amino acid sequence SEQ ID NO: 39, the gene comprising or consisting of the nucleic acid sequence SEQ ID NO: 17 codes for the protein comprising or consisting of the amino acid sequence SEQ ID NO: 40, the gene comprising or consisting of the nucleic acid sequence SEQ ID NO: 18 codes for the protein comprising or consisting of the amino acid sequence SEQ ID NO: 41, the gene comprising or consisting of the nucleic acid sequence SEQ ID NO: 19 codes for the protein comprising or consisting of the amino acid sequence SEQ ID NO: 42, the gene comprising or consisting of the nucleic acid sequence SEQ ID NO: 20 codes for the protein comprising or consisting of the amino acid sequence SEQ ID NO: 43, the gene comprising or consisting of the nucleic acid sequence SEQ ID NO: 21 codes for the protein comprising or consisting of the amino acid sequence SEQ ID NO: 44, the gene comprising or consisting of the nucleic acid sequence SEQ ID NO: 22 codes for the protein comprising or consisting of the amino acid sequence SEQ ID NO: 45 and the gene comprising or consisting of the nucleic acid sequence SEQ ID NO: 23 codes for the protein comprising or consisting of the amino acid sequence SEQ ID NO: 46.

The 23 genes according to the invention code for 23 proteins. The 23 proteins have been classified by the Inventors in two sets:
a. a first set of 4 proteins, expressed or present in expressed in BAS,
b. a second set of 3 proteins, expressed in ADK,
c. a third set of 6 proteins, expressed in SQC, and
d. a fourth set of 10 proteins expressed in LCNE.

The first set, also called in the invention set AP, of 4 proteins consists of the proteins comprising or constituted by the amino acid sequences SEQ ID NO: 24, SEQ ID NO: 25, SEQ ID NO: 26 and SEQ ID NO: 27.

The second set, also called in the invention set BP, of 3 proteins consists of the proteins comprising or constituted by the amino acid sequences SEQ ID NO: 28, SEQ ID NO: 29 and SEQ ID NO: 30.

The third set, also called in the invention set BP, of 6 proteins consists of the proteins comprising or constituted by the amino acid sequences SEQ ID NO: 31, SEQ ID NO: 32, SEQ ID NO: 33, SEQ ID NO: 34, SEQ ID NO: 35 and SEQ ID NO: 36.

The fourth set, also called in the invention set BP, of 10 proteins consists of the proteins comprising or constituted by the amino acid sequences SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46.

The inventors have also defined subsets of each of the above sets AP and BP as follows:

Set AP is divided into 4 subsets AP1, AP5, and AP6, said subset being such that:
a. subset AP1 consists of the proteins comprising or being constituted by the amino acid sequences SEQ ID NO: 24 and SEQ ID NO: 25,
b. subset AP5 consists of the protein comprising or being constituted by the amino acid sequence SEQ ID NO: 26, and
c. subset AP6 consists of the proteins comprising or being constituted by the amino acid sequences SEQ ID NO: 27.
   Set AP can also be divided into the following subsets:
a. subset AP1 consists of the proteins comprising or being constituted by the amino acid sequences SEQ ID NO: 24 and SEQ ID NO: 25,
b. subset AP2 consists of the proteins comprising or being constituted by the amino acid sequences SEQ ID NO: 24, SEQ ID NO: 25 and SEQ ID NO: 26,
c. subset AP3 consists of the proteins comprising or being constituted by the amino acid sequences SEQ ID NO: 24, SEQ ID NO: 25, SEQ ID NO: 26 and SEQ ID NO: 27.
   Set BP is divided into 3 subsets BP1 and BP3, said subset being such that:
a. subset BP1 consists of the proteins comprising or being constituted by the amino acid sequences SEQ ID NO: 28 and SEQ ID NO: 29, and
b. subset BP3 consists of the proteins comprising or being constituted by the amino acid sequences SEQ ID NO: 30.
   Set BP can also be divided into the following subsets:
a. subset BP1 consists of the proteins comprising or being constituted by the amino acid sequences SEQ ID NO: 28 and SEQ ID NO: 29, and
b. subset BP2 consists of the proteins comprising or being constituted by the amino acid sequences SEQ ID NO: 28, SEQ ID NO: 29 SEQ ID NO: 30.
   Set CP is divided into 3 subsets CP1, CP4 and CP5, said subset being such that:
a. subset CP1 consists of the proteins comprising or being constituted by the amino acid sequences SEQ ID NO: 31 and SEQ ID NO: 32,
b. subset CP4 consists of the proteins comprising or being constituted by the amino acid sequences SEQ ID NO: 33 and SEQ ID NO: 34,
c. subset CP5 consists of the proteins comprising or being constituted by the amino acid sequences SEQ ID NO: 35 and SEQ ID NO: 36.
   Set CP can also be divided into the following subsets:
a. subset CP1 consists of the proteins comprising or being constituted by the amino acid sequences SEQ ID NO: 31 and SEQ ID NO: 32,
b. subset CP2 consists of the proteins comprising or being constituted by the amino acid sequences SEQ ID NO: 31, SEQ ID NO: 32, SEQ ID NO: 33 and SEQ ID NO: 34, and
c. subset CP3 consists of the proteins comprising or being constituted by the amino acid sequences SEQ ID NO: 31, SEQ ID NO: 32, SEQ ID NO: 33, SEQ ID NO: 34, SEQ ID NO: 35 and SEQ ID NO: 36.
   Set DP is divided into 4 subsets DP1, DP5, DP6, DP7, said subset being such that:
a. subset DP1 consists of the proteins comprising or being constituted by the amino acid sequences SEQ ID NO: 37 and SEQ ID NO: 38,
b. subset DP5 consists of the proteins comprising or being constituted by the amino acid sequences SEQ ID NO: 39, SEQ ID NO: 40, SEQ ID NO: 41, SEQ ID NO: 42 and SEQ ID NO: 43,
c. subset DP6 consists of the proteins comprising or being constituted by the amino acid sequences SEQ ID NO: 44, and
d. subset DP7 consists of the proteins comprising or being constituted by the amino acid sequences SEQ ID NO: 45 and SEQ ID NO: 46
   Set DP can also be divided into the following subsets:
a. subset DP1 consists of the proteins comprising or being constituted by the amino acid sequences SEQ ID NO: 37 and SEQ ID NO: 38,
b. subset DP2 consists of the proteins comprising or being constituted by the amino acid sequences SEQ ID NO: 37, SEQ ID NO: 38, SEQ ID NO: 39, SEQ ID NO: 40, SEQ ID NO: 41, SEQ ID NO: 42 and SEQ ID NO: 43,
c. subset DP3 consists of the proteins comprising or being constituted by the amino acid sequences SEQ ID NO: 37, SEQ ID NO: 38, SEQ ID NO: 39, SEQ ID NO: 40, SEQ ID NO: 41, SEQ ID NO: 42, SEQ ID NO: 43 and SEQ ID NO: 44.
d. subset DP4 consists of the proteins comprising or being constituted by the amino acid sequences SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46.

In one advantageous embodiment, the invention relates to the use as defined above of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first subset A1 of 2 genes comprising or consisting of the nucleic acid sequences SEQ ID NO: 1 to 2
   b. at least one gene belongs to a second subset B1 of 2 genes comprising or consisting of the nucleic acid sequences SEQ ID NO: 5 to 6
   c. at least one gene belongs to a third subset C1 of 2 genes comprising or consisting of the nucleic acid sequences SEQ ID NO: 8 to 9, and
   d. at least one gene belongs to a fourth subset D1 of 2 genes comprising or consisting of the nucleic acid sequences SEQ ID NO: 14 to 15
at least 4 proteins being such that
   a. at least one protein belongs to a first subset AP1 of 2 proteins comprising or consisting of the amino acid sequences SEQ ID NO: 24 to 25,
   b. at least one protein belongs to a second subset BP1 of 2 proteins comprising or consisting of the amino acid sequences SEQ ID NO: 28 to 29
   c. at least one protein belongs to a second subset CP1 of 2 proteins comprising or consisting of the amino acid sequences SEQ ID NO: 31 to 32,
   d. at least one protein belongs to a second set DP1 of 2 proteins comprising or consisting of the amino acid sequences SEQ ID NO: 37 to 38,
at least 4 antibodies directed against said 4 proteins being such that
   a. at least one antibody specifically recognises one protein that belongs to a first subset AP1 of 2 proteins comprising or consisting of the amino acid sequences SEQ ID NO: 24 to 25,
   b. at least one antibody specifically recognises one protein that belongs to a second set BP1 of 2 proteins comprising or consisting of the amino acid sequences SEQ ID NO: 27 to 28,
   c. at least one antibody specifically recognises one protein that belongs to a first subset CP1 of 2 proteins comprising or consisting of the amino acid sequences SEQ ID NO: 31 to 32,
   d. at least one antibody specifically recognises one protein that belongs to a first subset DP1 of 2 proteins comprising or consisting of the amino acid sequences SEQ ID NO: 37 to 38,
      for the implementation of a prognosis method, preferably in vitro, of the survival rate of a patient afflicted by a lung cancer, said prognosis being such that: either
if none of the genes of said subset A1, B1, C1 and D1 is expressed, the patient survival rate during a period of time from 30 to 120 months after the diagnosis of said lung cancer is from about 59% to about 78% or more, and
if at least one gene of at least one subset A1, B, C1 or D1 is expressed, the patient survival rate during a period of time from 30 to 120 months after the diagnosis of said lung cancer is about from about 3% to about 70%,
   or
if none of the proteins of said subset AP1, BP1, CP1 and DP1 is expressed, the patient survival rate during a period of time from 30 to 120 months after the diagnosis of said lung cancer is from about 59% to about 78% or more, and
if at least one protein of at least one subset AP1, BP1, CP1 and DP1 is expressed, the patient survival rate during a period of time from 30 to 120 months after the diagnosis of said lung cancer is about from about 3% to about 70%,
   or
if none of the antibodies directed against the proteins of said subset AP1, BP1, CP1 and DP1 is expressed, the patient survival rate during a period of time from 30 to 120 months after the diagnosis of said lung cancer is from about 59% to about 78% or more, and
if at least one antibody directed against one protein of said subset AP1, BP1, CP1 and DP1 is expressed, the patient survival rate during a period of time from 30 to 120 months after the diagnosis of said lung cancer is about from about 3% to about 70%.

In this advantageous embodiment, the prognosis method according to the invention can be carried out as by measuring at least the expression of the following 6 combinations of proteins: SEQ ID NO: 24+28+31+37, SEQ ID NO: 24+28+31+38, SEQ ID NO: 24+28+32+37, SEQ ID NO: 24+28+32+38, SEQ ID NO: 24+29+31+37, SEQ ID NO: 24+29+31+38, SEQ ID NO: 24+29+32+37, SEQ ID NO: 24+29+32+38, SEQ ID NO: 25+28+31+37, SEQ ID NO: 25+28+31+38, SEQ ID NO: 25+28+32+37, SEQ ID NO: 25+28+32+38, SEQ ID NO: 25+29+31+37, SEQ ID NO: 25+29+31+38, SEQ ID NO: 25+29+32+37 and SEQ ID NO: 25+29+32+38.

For instance, the prognosis method according to the invention can be carried out by determining the expression status of the above combination SEQ ID NO: 24+SEQ ID NO: 28+SEQ ID NO: 32+SEQ ID NO: 37, wherein:
if none of the genes SEQ ID NO: 1, SEQ ID NO: 5, SEQ ID NO: 8 and SEQ ID NO: 15 is expressed, the patient survival rate during a period of time from 30 to 120 months after the diagnosis of said lung cancer is from about 59% to about 78% or more, and
if at least one gene SEQ ID NO: 1, SEQ ID NO: 5, SEQ ID NO: 8 or SEQ ID NO: 15 is expressed, the patient survival rate during a period of time from 30 to 120 months after the diagnosis of said lung cancer is about from about 3% to about 70%.

Moreover, the prognosis method according to the invention can be carried out as by measuring at least the expression of the following 16 combinations of proteins: SEQ ID NO: 24+25+31+37, SEQ ID NO: 24+25+31+38, SEQ ID NO: 24+25+32+37, SEQ ID NO: 24+25+32+38, SEQ ID NO: 24+29+31+37, SEQ ID NO: 24+29+31+38, SEQ ID NO: 24+29+32+37, SEQ ID NO: 24+29+32+38, SEQ ID NO: 25+25+31+37, SEQ ID NO: 25+25+31+38, SEQ ID NO: 25+25+32+37, SEQ ID NO: 25+25+32+38, SEQ ID NO: 25+29+31+37, SEQ ID NO: 25+29+31+38, SEQ ID NO: 25+29+32+37 and SEQ ID NO: 25+29+32+38.

For instance, the prognosis method according to the invention can be carried out by determining the expression status of the above combination SEQ ID NO: 24+SEQ ID NO: 5+SEQ ID NO: 8+SEQ ID NO: 15, wherein:
if none of the proteins SEQ ID NO: 24, SEQ ID NO: 28, SEQ ID NO: 32 and SEQ ID NO: 37 is expressed, the patient survival rate during a period of time from 30 to 120 months after the diagnosis of said lung cancer is from about 59% to about 78% or more, and
if at least one protein SEQ ID NO: 24, SEQ ID NO: 28, SEQ ID NO: 32 or SEQ ID NO: 37 is expressed, the patient survival rate during a period of time from 30 to 120 months after the diagnosis of said lung cancer is about from about 3% to about 70%.

Another advantageous embodiment of the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
   said
at least 4 genes being such that
   a. at least one gene belongs to a first subset A1 of 2 genes comprising or consisting of the nucleic acid sequences SEQ ID NO: 1 to 2
   b. at least one gene belongs to a second subset B1 of 2 genes comprising or consisting of the nucleic acid sequences SEQ ID NO: 5 to 6
   c. at least one gene belongs to a third subset C1 of 2 genes comprising or consisting of the nucleic acid sequences SEQ ID NO: 8 to 9, and
   d. at least one gene belongs to a fourth subset D1 of 2 genes comprising or consisting of the nucleic acid sequences SEQ ID NO: 14 to 15
      for the implementation of a prognosis method, preferably in vitro, of the survival rate of a patient afflicted by a lung cancer, said prognosis being such that:
if none of the genes of said subset A1, B1, C1 and D1 is expressed, the patient survival rate during a period of time from 30 to 120 months after the diagnosis of said lung cancer is from about 59% to about 78% or more, and
if at least one gene of at least one subset A1, B, C1 or D1 is expressed, the patient survival rate during a period of time from 30 to 120 months after the diagnosis of said lung cancer is about from about 3% to about 70%.

Another advantageous embodiment of the invention relates to the use of
proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 proteins being such that
   a. at least one protein belongs to a first subset AP1 of 2 proteins comprising or consisting of the amino acid sequences SEQ ID NO: 24 to 25,
   b. at least one protein belongs to a second subset BP1 of 2 proteins comprising or consisting of the amino acid sequences SEQ ID NO: 28 to 29
   c. at least one protein belongs to a second subset CP1 of 2 proteins comprising or consisting of the amino acid sequences SEQ ID NO: 31 to 32,
   d. at least one protein belongs to a second set DP1 of 2 proteins comprising or consisting of the amino acid sequences SEQ ID NO: 37 to 38,
      for the implementation of a prognosis method, preferably in vitro, of the survival rate of a patient afflicted by a lung cancer, said prognosis being such that:
if none of the proteins of said subset AP1, BP1, CP1 and DP1 is expressed, the patient survival rate during a period of time from 30 to 120 months after the diagnosis of said lung cancer is from about 59% to about 78% or more, and
if at least one protein of at least one subset AP1, BP1, CP1 and DP1 is expressed, the patient survival rate during a period of time from 30 to 120 months after the diagnosis of said lung cancer is about from about 3% to about 70%.

In one advantageous embodiment, the invention relates to the use as defined above of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first subset A4 comprising or consisting of the nucleic acid sequences SEQ ID NO: 3
   b. at least one gene belongs to a second subset B1 of 2 genes comprising or consisting of the nucleic acid sequences SEQ ID NO: 5 to 6
   c. at least one gene belongs to a third subset C4 of 2 genes comprising or consisting of the nucleic acid sequences SEQ ID NO: 10 to 11, and
   d. at least one gene belongs to a fourth subset D5 of 2 genes comprising or consisting of the nucleic acid sequences SEQ ID NO: 16 to 19
at least 4 proteins being such that
   a. at least one protein belongs to a first subset AP4 comprising or consisting of the amino acid sequences SEQ ID NO: 26,
   b. at least one protein belongs to a second subset BP1 of 2 proteins comprising or consisting of the amino acid sequences SEQ ID NO: 33 to 34
   c. at least one protein belongs to a second subset CP4 of 5 proteins comprising or consisting of the amino acid sequences SEQ ID NO: 37 to 38,
   d. at least one protein belongs to a second set DP5 of 2 proteins comprising or consisting of the amino acid sequences SEQ ID NO: 39 to 43,
at least 4 antibodies directed against said 4 proteins being such that
   a. at least one antibody specifically recognises one protein that belongs to a first subset AP4 comprising or consisting of the amino acid sequences SEQ ID NO: 26,
   b. at least one antibody specifically recognises one protein that belongs to a second set BP1 of 2 proteins comprising or consisting of the amino acid sequences SEQ ID NO: 33 to 34,
   c. at least one antibody specifically recognises one protein that belongs to a first subset CP4 of 2 proteins comprising or consisting of the amino acid sequences SEQ ID NO: 31 to 32,
   d. at least one antibody specifically recognises one protein that belongs to a first subset DP1 of 2 proteins comprising or consisting of the amino acid sequences SEQ ID NO: 39 to 43,
      for the implementation of a prognosis method, preferably in vitro, of the survival rate of a patient afflicted by a lung cancer, said prognosis being such that:
      either
if none of the genes of said subset A4, B1, C4 and D5 is expressed, the patient survival rate during a period of time from 30 to 120 months after the diagnosis of said lung cancer is from about 59% to about 78% or more, and
if at least one gene of at least one subset A4, B, C4 or D5 is expressed, the patient survival rate during a period of time from 30 to 120 months after the diagnosis of said lung cancer is about from about 3% to about 70%,
   or
if none of the proteins of said subset AP4, BP1, CP4 and DP5 is expressed, the patient survival rate during a period of time from 30 to 120 months after the diagnosis of said lung cancer is from about 59% to about 78% or more, and
if at least one protein of at least one subset AP4, BP1, CP4 and DP5 is expressed, the patient survival rate during a period of time from 30 to 120 months after the diagnosis of said lung cancer is about from about 3% to about 70%,
   or
if none of the antibodies directed against the proteins of said subset AP4, BP1, CP4 and DP5 is expressed, the patient survival rate during a period of time from 30 to 120 months after the diagnosis of said lung cancer is from about 59% to about 78% or more, and
if at least one antibody directed against one protein of said subset AP4, BP1, CP4 and DP5 is expressed, the patient survival rate during a period of time from 30 to 120 months after the diagnosis of said lung cancer is about from about 3% to about 70%.

Another advantageous embodiment of the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
   said
at least 4 genes being such that
   a. at least one gene belongs to a first subset A4 comprising or consisting of the nucleic acid sequences SEQ ID NO: 3
   b. at least one gene belongs to a second subset B1 of 2 genes comprising or consisting of the nucleic acid sequences SEQ ID NO: 5 to 6
   c. at least one gene belongs to a third subset C4 of 2 genes comprising or consisting of the nucleic acid sequences SEQ ID NO: 10 to 11, and
   d. at least one gene belongs to a fourth subset D5 of 2 genes comprising or consisting of the nucleic acid sequences SEQ ID NO: 16 to 19
      for the implementation of a prognosis method, preferably in vitro, of the survival rate of a patient afflicted by a lung cancer, said prognosis being such that:
if none of the genes of said subset A4, B1, C4 and D5 is expressed, the patient survival rate during a period of time from 30 to 120 months after the diagnosis of said lung cancer is from about 59% to about 78% or more, and
if at least one gene of at least one subset A4, B1, C4 or D5 is expressed, the patient survival rate during a period of time from 30 to 120 months after the diagnosis of said lung cancer is about from about 3% to about 70%,

Another advantageous embodiment of the invention relates to the use of
proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 proteins being such that
at least 4 proteins being such that
   e. at least one protein belongs to a first subset AP4 comprising or consisting of the amino acid sequences SEQ ID NO: 26,
   f. at least one protein belongs to a second subset BP1 of 2 proteins comprising or consisting of the amino acid sequences SEQ ID NO: 33 to 34
   g. at least one protein belongs to a second subset CP4 of 5 proteins comprising or consisting of the amino acid sequences SEQ ID NO: 37 to 38,
   h. at least one protein belongs to a second set DP5 of 2 proteins comprising or consisting of the amino acid sequences SEQ ID NO: 39 to 43,
      for the implementation of a prognosis method, preferably in vitro, of the survival rate of a patient afflicted by a lung cancer, said prognosis being such that:
if none of the proteins of said subset AP4, BP1, CP4 and DP5 is expressed, the patient survival rate during a period of time from 30 to 120 months after the diagnosis of said lung cancer is from about 59% to about 78% or more, and
if at least one protein of at least one subset AP4, BP1, CP4 and DP5 is expressed, the patient survival rate during a period of time from 30 to 120 months after the diagnosis of said lung cancer is about from about 3% to about 70%.

In one advantageous embodiment, the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first subset A2 of 3 genes comprising or consisting of the nucleic acid sequences SEQ ID NO: 1 to 3
   b. at least one gene belongs to a second subset B1 of 2 genes comprising or consisting of the nucleic acid sequences SEQ ID NO: 5 to 6
   c. at least one gene belongs to a third subset C2 of 4 genes comprising or consisting of the nucleic acid sequences SEQ ID NO: 8 to 11, and
   d. at least one gene belongs to a fourth subset D2 of 7 genes comprising or consisting of the nucleic acid sequences SEQ ID NO: 14 to 20
at least 4 proteins being such that
   a. at least one protein belongs to a first subset AP2 of 3 proteins comprising or consisting of the amino acid sequences SEQ ID NO: 24 to 26,
   b. at least one protein belongs to a second subset BP1 of 2 proteins comprising or consisting of the amino acid sequences SEQ ID NO: 28 to 29
   c. at least one protein belongs to a second subset CP2 of 4 proteins comprising or consisting of the amino acid sequences SEQ ID NO: 31 to 34,
   d. at least one protein belongs to a second set DP2 of 7 proteins comprising or consisting of the amino acid sequences SEQ ID NO: 37 to 43,
at least 4 antibodies directed against said 4 proteins being such that
   a. at least one antibody specifically recognises one protein that belongs to a first subset AP2 of 3 proteins comprising or consisting of the amino acid sequences SEQ ID NO: 24 to 26,
   b. at least one antibody specifically recognises one protein that belongs to a second set BP1 of 4 proteins comprising or consisting of the amino acid sequences SEQ ID NO: 27 to 28,
   c. at least one antibody specifically recognises one protein that belongs to a first subset CP2 of 4 proteins comprising or consisting of the amino acid sequences SEQ ID NO: 31 to 34,
   d. at least one antibody specifically recognises one protein that belongs to a first subset DP2 of 7 proteins comprising or consisting of the amino acid sequences SEQ ID NO: 37 to 43,
      for the implementation of a prognosis method, preferably in vitro, of the survival rate of a patient afflicted by a lung cancer, said prognosis being such that:
      either
if none of the genes of said subset A2, B1, C2 and D2 is expressed, the patient survival rate during a period of time from 30 to 120 months after the diagnosis of said lung cancer is from about 59% to about 78% or more, and
if at least one gene of at least one subset A2, B1, C2 or D2 is expressed, the patient survival rate during a period of time from 30 to 120 months after the diagnosis of said lung cancer is about from about 3% to about 70%,
   or
if none of the proteins of said subset AP2, BP1, CP2 and DP2 is expressed, the patient survival rate during a period of time from 30 to 120 months after the diagnosis of said lung cancer is from about 59% to about 78% or more, and
if at least one protein of at least one subset AP2, BP1, CP2 and DP2 is expressed, the patient survival rate during a period of time from 30 to 120 months after the diagnosis of said lung cancer is about from about 3% to about 70%,
   or
if none of the antibodies directed against the proteins of said subset AP2, BP1, CP2 and DP2 is expressed, the patient survival rate during a period of time from 30 to 120 months after the diagnosis of said lung cancer is from about 59% to about 78% or more, and
if at least one antibody directed against one protein of said subset AP2, BP1, CP2 and DP2 is expressed, the patient survival rate during a period of time from 30 to 120 months after the diagnosis of said lung cancer is about from about 3% to about 70%.

Another advantageous embodiment of the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
   said
at least 4 genes being such that
   a. at least one gene belongs to a first subset A2 of 3 genes comprising or consisting of the nucleic acid sequences SEQ ID NO: 1 to 3
   b. at least one gene belongs to a second subset B1 of 2 genes comprising or consisting of the nucleic acid sequences SEQ ID NO: 5 to 6
   c. at least one gene belongs to a third subset C2 of 4 genes comprising or consisting of the nucleic acid sequences SEQ ID NO: 8 to 11, and
   d. at least one gene belongs to a fourth subset D2 of 7 genes comprising or consisting of the nucleic acid sequences SEQ ID NO: 14 to 20
      for the implementation of a prognosis method, preferably in vitro, of the survival rate of a patient afflicted by a lung cancer, said prognosis being such that:
if none of the genes of said subset A2, B1, C2 and D21 is expressed, the patient survival rate during a period of time from 30 to 120 months after the diagnosis of said lung cancer is from about 59% to about 78% or more, and
if at least one gene of at least one subset A2, B1, C2 or D2 is expressed, the patient survival rate during a period of time from 30 to 120 months after the diagnosis of said lung cancer is about from about 3% to about 70%.

Another advantageous embodiment of the invention relates to the use of
proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 proteins being such that
   a. at least one protein belongs to a first subset AP2 of 3 proteins comprising or consisting of the amino acid sequences SEQ ID NO: 24 to 26,
   b. at least one protein belongs to a second subset BP1 of 2 proteins comprising or consisting of the amino acid sequences SEQ ID NO: 28 to 29
   c. at least one protein belongs to a second subset CP2 of 4 proteins comprising or consisting of the amino acid sequences SEQ ID NO: 31 to 34,
   d. at least one protein belongs to a second set DP2 of 7 proteins comprising or consisting of the amino acid sequences SEQ ID NO: 37 to 43,
      for the implementation of a prognosis method, preferably in vitro, of the survival rate of a patient afflicted by a lung cancer, said prognosis being such that:
if none of the proteins of said subset AP2, BP1, CP2 and DP2 is expressed, the patient survival rate during a period of time from 30 to 120 months after the diagnosis of said lung cancer is from about 59% to about 78% or more, and
if at least one protein of at least one subset AP2, BP1, CP2 and DP2 is expressed, the patient survival rate during a period of time from 30 to 120 months after the diagnosis of said lung cancer is about from about 3% to about 70%.

In one advantageous embodiment, the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first subset A2 of 3 genes comprising or consisting of the nucleic acid sequences SEQ ID NO: 1 to 3
   b. at least one gene belongs to a second subset B1 of 2 genes comprising or consisting of the nucleic acid sequences SEQ ID NO: 5 to 6
   c. at least one gene belongs to a third subset C3 of 6 genes comprising or consisting of the nucleic acid sequences SEQ ID NO: 8 to 13, and
   d. at least one gene belongs to a fourth subset D3 of 8 genes comprising or consisting of the nucleic acid sequences SEQ ID NO: 14 to 21,
at least 4 proteins being such that
   a. at least one protein belongs to a first subset AP2 of 3 proteins comprising or consisting of the amino acid sequences SEQ ID NO: 24 to 26,
   b. at least one protein belongs to a second subset BP1 of 2 proteins comprising or consisting of the amino acid sequences SEQ ID NO: 28 to 29
   c. at least one protein belongs to a second subset CP3 of 6 proteins comprising or consisting of the amino acid sequences SEQ ID NO: 31 to 36,
   d. at least one protein belongs to a second set DP3 of 8 proteins comprising or consisting of the amino acid sequences SEQ ID NO: 37 to 44,
at least 4 antibodies directed against said 4 proteins being such that
   a. at least one antibody specifically recognises one protein that belongs to a first subset AP2 of 3 proteins comprising or consisting of the amino acid sequences SEQ ID NO: 24 to 26,
   b. at least one antibody specifically recognises one protein that belongs to a second set BP1 of 4 proteins comprising or consisting of the amino acid sequences SEQ ID NO: 27 to 28,
   c. at least one antibody specifically recognises one protein that belongs to a first subset CP3 of 6 proteins comprising or consisting of the amino acid sequences SEQ ID NO: 31 to 36,
   d. at least one antibody specifically recognises one protein that belongs to a first subset DP3 of 8 proteins comprising or consisting of the amino acid sequences SEQ ID NO: 37 to 44,
      for the implementation of a prognosis method, preferably in vitro, of the survival rate of a patient afflicted by a lung cancer, said prognosis being such that:
      either
if none of the genes of said subset A2, B1, C3 and D3 is expressed, the patient survival rate during a period of time from 30 to 120 months after the diagnosis of said lung cancer is from about 59% to about 78% or more, and
if at least one gene of at least one subset A2, B1, C3 or D3 is expressed, the patient survival rate during a period of time from 30 to 120 months after the diagnosis of said lung cancer is about from about 3% to about 70%,
   or
if none of the proteins of said subset AP2, BP1, CP3 and DP3 is expressed, the patient survival rate during a period of time from 30 to 120 months after the diagnosis of said lung cancer is from about 59% to about 78% or more, and
if at least one protein of at least one subset AP2, BP1, CP3 and DP3 is expressed, the patient survival rate during a period of time from 30 to 120 months after the diagnosis of said lung cancer is about from about 3% to about 70%,
   or
if none of the antibodies directed against the proteins of said subset AP2, BP1, CP3 and DP3 is expressed, the patient survival rate during a period of time from 30 to 120 months after the diagnosis of said lung cancer is from about 59% to about 78% or more, and
if at least one antibody directed against one protein of said subset AP2, BP1, CP3 and DP3 is expressed, the patient survival rate during a period of time from 30 to 120 months after the diagnosis of said lung cancer is about from about 3% to about 70%.

Another advantageous embodiment of the invention relates to the use of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
   said
at least 4 genes being such that
   a. at least one gene belongs to a first subset A2 of 3 genes comprising or consisting of the nucleic acid sequences SEQ ID NO: 1 to 3
   b. at least one gene belongs to a second subset B1 of 2 genes comprising or consisting of the nucleic acid sequences SEQ ID NO: 5 to 6
   c. at least one gene belongs to a third subset C3 of 6 genes comprising or consisting of the nucleic acid sequences SEQ ID NO: 8 to 13, and
   d. at least one gene belongs to a fourth subset D3 of 8 genes comprising or consisting of the nucleic acid sequences SEQ ID NO: 14 to 21,
      for the implementation of a prognosis method, preferably in vitro, of the survival rate of a patient afflicted by a lung cancer, said prognosis being such that:
if none of the genes of said subset A2, B1, C3 and D3 is expressed, the patient survival rate during a period of time from 30 to 120 months after the diagnosis of said lung cancer is from about 59% to about 78% or more, and
if at least one gene of at least one subset A2, B1, C3 or D3 is expressed, the patient survival rate during a period of time from 30 to 120 months after the diagnosis of said lung cancer is about from about 3% to about 70%.

Another advantageous embodiment of the invention relates to the use of
proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 proteins being such that
   a. at least one protein belongs to a first subset AP2 of 3 proteins comprising or consisting of the amino acid sequences SEQ ID NO: 24 to 26,
   b. at least one protein belongs to a second subset BP1 of 2 proteins comprising or consisting of the amino acid sequences SEQ ID NO: 28 to 29
   c. at least one protein belongs to a second subset CP3 of 6 proteins comprising or consisting of the amino acid sequences SEQ ID NO: 31 to 36,
   d. at least one protein belongs to a second set DP3 of 8 proteins comprising or consisting of the amino acid sequences SEQ ID NO: 37 to 44,
      for the implementation of a prognosis method, preferably in vitro, of the survival rate of a patient afflicted by a lung cancer, said prognosis being such that:
if none of the proteins of said subset AP2, BP1, CP3 and DP3 is expressed, the patient survival rate during a period of time from 30 to 120 months after the diagnosis of said lung cancer is from about 59% to about 78% or more, and
if at least one protein of at least one subset AP2, BP1, CP3 and DP3 is expressed, the patient survival rate during a period of time from 30 to 120 months after the diagnosis of said lung cancer is about from about 3% to about 70%,

In one other embodiment, the invention relates to the use according as defined above, wherein said prognosis is such that
when said tumor is ADK:
   if none of the genes of said set B or of the subset B1 is expressed, the patient survival rate is from about 76% to about 86%
   if at least one of the genes of the set B or of the subset B1 is expressed, the patient survival rate is from about 39% to about 63%,
or
   if none of the proteins of said set BP or of the subset BP1 is expressed, the patient survival rate is from about 76% to about 86%
   if at least one of the proteins of the set BP1 or of the subset BP1 is expressed, the patient survival rate is from about 39% to about 63%,
or
   if none of antibodies directed against the proteins of said set BP or of the subset BP1 is expressed, the patient survival rate is from about 76% to about 86%
   if at least one antibody directed against at least one of the proteins of the set BP or of the subset BP1 is expressed, the patient survival rate is from about 39% to about 63%,
when said tumor is BAS:
   if none of the genes of the set A or of subsets A1 or A2 is expressed, the patient survival rate is about 19% to about 65%
   if at least one of the genes of the set A or of subsets A1 or A2 is expressed, the patient survival rate is about 0% to about 33%,
or
   if none of the proteins of the set AP or of subsets AP1 or AP2 is expressed, the patient survival rate is about 19% to about 65%
   if at least one of the proteins of the set AP or of subsets AP1 or AP2 is expressed, the patient survival rate is about 0% to about 33%,
or
   if none of antibodies directed against the proteins of said set AP or of subsets AP1 or AP2 is expressed, the patient survival rate is about 19% to about 65%
   if at least one antibody directed against at least one of the proteins of the set AP or of subsets AP1 or AP2 is expressed, the patient survival rate is about 0% to about 33%,
when said tumor is SQC:
   if none of the genes of the set C or of the subsets C1 or C2 is expressed, the patient survival rate is about 58% to about 88%
   if at least one of the genes of the set C or of the subsets C1 or C2 is expressed, the patient survival rate is from about 33% to about 64%,
or
   if none of the proteins of the set CP or of the subsets CP1 or CP2 is expressed, the patient survival rate is about 58% to about 88%
   if at least one of the proteins of the set CP or of the subsets CP1 or CP2 is expressed, the patient survival rate is from about 33% to about 64%,
or
   if none of antibodies directed against the proteins of said set CP or of the subsets CP1 or CP2 is expressed, the patient survival rate is about 58% to about 88%
   if at least one antibody directed against at least one of the proteins of the set CP or of the subsets CP1 or CP2 is expressed, the patient survival rate is from about 33% to about 64%,
when said tumor is LCNE:
   if none of the genes of the set D or of the subsets D1, D2 or D3 is expressed, the patient survival rate is from about 40% to about 65%
   if at least one of the genes of the set D or of the subsets D1, D2 or D3 is expressed, the patient survival rate is from about 0% to 7%,
or
   if none of the proteins of the set DP or of the subsets DP1, DP2 or DP3 is expressed, the patient survival rate is from about 40% to about 65%
   if at least one of the proteins of the set DP or of the subsets DP1, DP2 or DP3 is expressed, the patient survival rate is from about 0% to 7%,
or
   if none of antibodies directed against the proteins of said set DP or of the subsets DP1, DP2 or DP3 is expressed, the patient survival rate is from about 40% to about 65%,
   if at least one antibody directed against at least one of the proteins of the set DP or of the subsets DP1, DP2 or DP3 is expressed, the patient survival rate is from about 0% to 7%.

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
or
   if none of the proteins of the subset BP1 is expressed, the patient survival rate is from about 76% to about 86%
   if at least one of the subset BP1 is expressed, the patient survival rate is from about 39% to about 63%,
or
   if none of antibodies directed against the proteins of the subset BP1 is expressed, the patient survival rate is from about 76% to about 86%
   if at least one antibody directed against at least one of the proteins of the subset BP1 is expressed, the patient survival rate is from about 39% to about 63%,
when said tumor is BAS:
   if none of the genes of subset A1 is expressed, the patient survival rate is about 19% to about 65%
   if at least one of the genes of subsets A1 is expressed, the patient survival rate is about 0% to about 33%,
or
   if none of the proteins of subsets AP1 is expressed, the patient survival rate is about 19% to about 65%
   if at least one of the proteins of the subsets AP1 or is expressed, the patient survival rate is about 0% to about 33%,
or
   if none of antibodies directed against the proteins of subset AP1 is expressed, the patient survival rate is about 19% to about 65%
   if at least one antibody directed against at least one of the proteins of the subset AP1 is expressed, the patient survival rate is about 0% to about 33%,
when said tumor is SQC:
   if none of the genes of the subset C1 is expressed, the patient survival rate is about 58% to about 88%
   if at least one of the genes of the subset C1 is expressed, the patient survival rate is from about 33% to about 64%,
or
   if none of the proteins of the subset CP1 is expressed, the patient survival rate is about 58% to about 88%
   if at least one of the proteins of the subset CP1 is expressed, the patient survival rate is from about 33% to about 64%,
or
   if none of antibodies directed against the proteins of the subsets CP1 is expressed, the patient survival rate is about 58% to about 88%
   if at least one antibody directed against at least one of the proteins of the subset CP1 is expressed, the patient survival rate is from about 33% to about 64%,
when said tumor is LCNE:
   if none of the genes of the subset D1 is expressed, the patient survival rate is from about 40% to about 65%
   if at least one of the genes of the subset D1 is expressed, the patient survival rate is from about 0% to 7%,
or
   if none of the proteins of the subset DP1 is expressed, the patient survival rate is from about 40% to about 65%
   if at least one of the proteins of the subset DP1 is expressed, the patient survival rate is from about 0% to 7%,
or
   if none of antibodies directed against the proteins of the subset DP1 is expressed, the patient survival rate is from about 40% to about 65%,
   if at least one antibody directed against at least one of the proteins of the subset DP1 is expressed, the patient survival rate is from about 0% to 7%.

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
when said tumor is BAS:
   if none of the genes of subset A1 is expressed, the patient survival rate is about 19% to about 65%
   if at least one of the genes of subsets A1 is expressed, the patient survival rate is about 0% to about 33%,
when said tumor is SQC:
   if none of the genes of the subset C1 is expressed, the patient survival rate is about 58% to about 88%
   if at least one of the genes of the subset C1 is expressed, the patient survival rate is from about 33% to about 64%,
when said tumor is LCNE:
   if none of the genes of the subset D1 is expressed, the patient survival rate is from about 40% to about 65%
   if at least one of the genes of the subset D1 is expressed, the patient survival rate is from about 0% to 7%.

In one other embodiment, the invention relates to the use according as defined above, wherein said prognosis is such that
when said tumor is ADK:
   if none of the proteins of the subset BP1 is expressed, the patient survival rate is from about 76% to about 86%
   if at least one of the subset BP1 is expressed, the patient survival rate is from about 39% to about 63%,
when said tumor is BAS:
   if none of the proteins of subsets AP1 is expressed, the patient survival rate is about 19% to about 65%
   if at least one of the proteins of the subsets AP1 or is expressed, the patient survival rate is about 0% to about 33%,
when said tumor is SQC:
   if none of the proteins of the subset CP1 is expressed, the patient survival rate is about 58% to about 88%
   if at least one of the proteins of the subset CP1 is expressed, the patient survival rate is from about 33% to about 64%,
when said tumor is LCNE:
   if none of the proteins of the subset DP1 is expressed, the patient survival rate is from about 40% to about 65%
   if at least one of the proteins of the subset DP1 is expressed, the patient survival rate is from about 0% to 7%,

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
or
   if none of the proteins of the subset BP1 is expressed, the patient survival rate is from about 76% to about 86%
   if at least one of the subset BP1 is expressed, the patient survival rate is from about 39% to about 63%,
or
   if none of antibodies directed against the proteins of the subset BP1 is expressed, the patient survival rate is from about 76% to about 86%
   if at least one antibody directed against at least one of the proteins of the subset BP1 is expressed, the patient survival rate is from about 39% to about 63%,
when said tumor is BAS:
   if none of the genes of subset A2 is expressed, the patient survival rate is about 19% to about 65%
   if at least one of the genes of subsets A2 is expressed, the patient survival rate is about 0% to about 33%,
or
   if none of the proteins of subsets AP2 is expressed, the patient survival rate is about 19% to about 65%
   if at least one of the proteins of the subsets AP2 or is expressed, the patient survival rate is about 0% to about 33%,
or
   if none of antibodies directed against the proteins of subset AP2 is expressed, the patient survival rate is about 19% to about 65%
   if at least one antibody directed against at least one of the proteins of the subset AP2 is expressed, the patient survival rate is about 0% to about 33%,
when said tumor is SQC:
   if none of the genes of the subset C2 is expressed, the patient survival rate is about 58% to about 88%
   if at least one of the genes of the subset C2 is expressed, the patient survival rate is from about 33% to about 64%,
or
   if none of the proteins of the subset CP2 is expressed, the patient survival rate is about 58% to about 88%
   if at least one of the proteins of the subset CP2 is expressed, the patient survival rate is from about 33% to about 64%,
or
   if none of antibodies directed against the proteins of the subsets CP2 is expressed, the patient survival rate is about 58% to about 88%
   if at least one antibody directed against at least one of the proteins of the subset CP2 is expressed, the patient survival rate is from about 33% to about 64%,
when said tumor is LCNE:
   if none of the genes of the subset D2 is expressed, the patient survival rate is from about 40% to about 65%
   if at least one of the genes of the subset D2 is expressed, the patient survival rate is from about 0% to 7%,
or
   if none of the proteins of the subset DP2 is expressed, the patient survival rate is from about 40% to about 65%
   if at least one of the proteins of the subset DP2 is expressed, the patient survival rate is from about 0% to 7%,
or
   if none of antibodies directed against the proteins of the subset DP2 is expressed, the patient survival rate is from about 40% to about 65%,
   if at least one antibody directed against at least one of the proteins of the subset DP2 is expressed, the patient survival rate is from about 0% to 7%.

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
when said tumor is BAS:
   if none of the genes of subset A2 is expressed, the patient survival rate is about 19% to about 65%
   if at least one of the genes of subsets A2 is expressed, the patient survival rate is about 0% to about 33%,
when said tumor is SQC:
   if none of the genes of the subset C2 is expressed, the patient survival rate is about 58% to about 88%
   if at least one of the genes of the subset C2 is expressed, the patient survival rate is from about 33% to about 64%,
when said tumor is LCNE:
   if none of the genes of the subset D2 is expressed, the patient survival rate is from about 40% to about 65%
   if at least one of the genes of the subset D2 is expressed, the patient survival rate is from about 0% to 7%.

In one other embodiment, the invention relates to the use according as defined above, wherein said prognosis is such that
when said tumor is ADK:
   if none of the proteins of the subset BP1 is expressed, the patient survival rate is from about 76% to about 86%
   if at least one of the subset BP1 is expressed, the patient survival rate is from about 39% to about 63%,
when said tumor is BAS:
   if none of the proteins of subsets AP2 is expressed, the patient survival rate is about 19% to about 65%
   if at least one of the proteins of the subsets AP2 or is expressed, the patient survival rate is about 0% to about 33%,
when said tumor is SQC:
   if none of the proteins of the subset CP2 is expressed, the patient survival rate is about 58% to about 88%
   if at least one of the proteins of the subset CP2 is expressed, the patient survival rate is from about 33% to about 64%,
when said tumor is LCNE:
   if none of the proteins of the subset DP2 is expressed, the patient survival rate is from about 40% to about 65%
   if at least one of the proteins of the subset DP2 is expressed, the patient survival rate is from about 0% to 7%.

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
or
   if none of the proteins of the subset BP1 is expressed, the patient survival rate is from about 76% to about 86%
   if at least one of the subset BP1 is expressed, the patient survival rate is from about 39% to about 63%,
or
   if none of antibodies directed against the proteins of the subset BP1 is expressed, the patient survival rate is from about 76% to about 86%
   if at least one antibody directed against at least one of the proteins of the subset BP1 is expressed, the patient survival rate is from about 39% to about 63%,
when said tumor is BAS:
   if none of the genes of subset A2 is expressed, the patient survival rate is about 19% to about 65%
   if at least one of the genes of subsets A2 is expressed, the patient survival rate is about 0% to about 33%,
or
   if none of the proteins of subsets AP2 is expressed, the patient survival rate is about 19% to about 65%
   if at least one of the proteins of the subsets AP2 or is expressed, the patient survival rate is about 0% to about 33%,
or
   if none of antibodies directed against the proteins of subset AP21 is expressed, the patient survival rate is about 19% to about 65%
   if at least one antibody directed against at least one of the proteins of the subset AP2 is expressed, the patient survival rate is about 0% to about 33%,
when said tumor is SQC:
   if none of the genes of the subset C2 is expressed, the patient survival rate is about 58% to about 88%
   if at least one of the genes of the subset C2 is expressed, the patient survival rate is from about 33% to about 64%,
or
   if none of the proteins of the subset CP2 is expressed, the patient survival rate is about 58% to about 88%
   if at least one of the proteins of the subset CP2 is expressed, the patient survival rate is from about 33% to about 64%,
or
   if none of antibodies directed against the proteins of the subsets CP2 is expressed, the patient survival rate is about 58% to about 88%
   if at least one antibody directed against at least one of the proteins of the subset CP2 is expressed, the patient survival rate is from about 33% to about 64%,
when said tumor is LCNE:
   if none of the genes of the subset D3 is expressed, the patient survival rate is from about 40% to about 65%
   if at least one of the genes of the subset D3 is expressed, the patient survival rate is from about 0% to 7%,
or
   if none of the proteins of the subset DP3 is expressed, the patient survival rate is from about 40% to about 65%
   if at least one of the proteins of the subset DP3 is expressed, the patient survival rate is from about 0% to 7%,
or
   if none of antibodies directed against the proteins of the subset DP3 is expressed, the patient survival rate is from about 40% to about 65%,
   if at least one antibody directed against at least one of the proteins of the subset DP3 is expressed, the patient survival rate is from about 0% to 7%.

In one other embodiment, the invention relates to the use according as defined above, wherein said prognosis is such that
when said tumor is ADK:
   if none of the genes of the B1 is expressed, the patient survival rate is from about 76% to about 86%
   if at least one of the genes of the subset B1 is expressed, the patient survival rate is from about 39% to about 63%,
when said tumor is BAS:
   if none of the genes of subset A2 is expressed, the patient survival rate is about 19% to about 65%
   if at least one of the genes of subsets A2 is expressed, the patient survival rate is about 0% to about 33%,
when said tumor is SQC:
   if none of the genes of the subset C2 is expressed, the patient survival rate is about 58% to about 88%
   if at least one of the genes of the subset C2 is expressed, the patient survival rate is from about 33% to about 64%,
when said tumor is LCNE:
   if none of the genes of the subset D3 is expressed, the patient survival rate is from about 40% to about 65%
   if at least one of the genes of the subset D3 is expressed, the patient survival rate is from about 0% to 7%.

In one other embodiment, the invention relates to the use according as defined above, wherein said prognosis is such that
when said tumor is ADK:
   if none of the proteins of the subset BP1 is expressed, the patient survival rate is from about 76% to about 86%
   if at least one of the subset BP1 is expressed, the patient survival rate is from about 39% to about 63%,
when said tumor is BAS:
   if none of the proteins of subsets AP2 is expressed, the patient survival rate is about 19% to about 65%
   if at least one of the proteins of the subsets AP2 or is expressed, the patient survival rate is about 0% to about 33%,
when said tumor is SQC:
   if none of the proteins of the subset CP2 is expressed, the patient survival rate is about 58% to about 88%
   if at least one of the proteins of the subset CP2 is expressed, the patient survival rate is from about 33% to about 64%,
when said tumor is LCNE:
   if none of the proteins of the subset DP3 is expressed, the patient survival rate is from about 40% to about 65%
   if at least one of the proteins of the subset DP3 is expressed, the patient survival rate is from about 0% to 7%.

In one particular embodiment, the invention relates to the use as defined above, wherein
when said tumor is SQC:
   if none of the genes of said set C or of subsets C1 and C2 is expressed, the patient survival rate is about 58% to about 88%
   if one of the genes of the set C or of subsets C1 and C2 is expressed, the patient survival rate is from about 45% to about 73%,
   if at least two of the genes of the set C or of subsets C1 and C2 are expressed, the patient survival rate is from about 14%, to about 50%,
or
   if none of the proteins of said set CP or of subsets CP1 and CP2 is expressed, the patient survival rate is about 58% to about 88%
   if one of the proteins of the set CP or of subsets CP1 and CP2 is expressed, the patient survival rate is from about 45% to about 73%,
   if at least two of the proteins of the set CP or of subsets CP1 and CP2 are expressed, the patient survival rate is from about 14%, to about 50%,
or
   if none of the antibodies directed against proteins said set CP or of subsets CP1 and CP2 is expressed, the patient survival rate is about 58% to about 88%,
   if one of antibody directed against one protein of the set CP or of subsets CP1 and CP2 is expressed, the patient survival rate is from about 45% to about 73%,
   if antibodies directed against at least two proteins of the set CP or of subsets CP1 and CP2 are expressed, the patient survival rate is from about 14%, to about 50%.

In one particular embodiment, the invention relates to the use as defined above, wherein
when said tumor is SQC:
   if none of the genes of said set C or of subsets C1 and C2 is expressed, the patient survival rate is about 58% to about 88%
   if one of the genes of the set C or of subsets C1 and C2 is expressed, the patient survival rate is from about 45% to about 73%,
   if at least two of the genes of the set C or of subsets C1 and C2 are expressed, the patient survival rate is from about 14%, to about 50%,

In one particular embodiment, the invention relates to the use as defined above, wherein
when said tumor is SQC:
   if none of the genes of said subset is expressed, the patient survival rate is about 58% to about 88%
   if one of the genes of the subset C1 is expressed, the patient survival rate is from about 45% to about 73%,
   if at least two of the genes of the subset C1 are expressed, the patient survival rate is from about 14%, to about 50%.

In one particular embodiment, the invention relates to the use as defined above, wherein
when said tumor is SQC:
   if none of the genes of said subset is expressed, the patient survival rate is about 58% to about 88%
   if one of the genes of the subset C2 is expressed, the patient survival rate is from about 45% to about 73%,
   if at least two of the genes of the subset C2 are expressed, the patient survival rate is from about 14%, to about 50%.

In one particular embodiment, the invention relates to the use as defined above, wherein
when said tumor is SQC:
   if none of the proteins of said set CP or of subsets CP1 and CP2 is expressed, the patient survival rate is about 58% to about 88%
   if one of the proteins of the set CP or of subsets CP1 and CP2 is expressed, the patient survival rate is from about 45% to about 73%,
   if at least two of the proteins of the set CP or of subsets CP1 and CP2 are expressed, the patient survival rate is from about 14%, to about 50%.

In one particular embodiment, the invention relates to the use as defined above, wherein
when said tumor is SQC:
   if none of the proteins of said subset CP1 is expressed, the patient survival rate is about 58% to about 88%
   if one of the proteins of the subset CP1 is expressed, the patient survival rate is from about 45% to about 73%,
   if at least two of the proteins of the subset CP1 are expressed, the patient survival rate is from about 14%, to about 50%.

In one particular embodiment, the invention relates to the use as defined above, wherein
when said tumor is SQC:
   if none of the proteins of said subset CP2 is expressed, the patient survival rate is about 58% to about 88%
   if one of the proteins of the subset CP2 is expressed, the patient survival rate is from about 45% to about 73%,
   if at least two of the proteins of the subset CP2 are expressed, the patient survival rate is from about 14%, to about 50%.

In another advantageous embodiment, the invention relates to the use as defined above, wherein during a period of time of 120 months from the diagnosis of said lung cancer,

wherein
when said tumor is ADK:
   if none of the genes of said set B or of the subset B1 is expressed, the patient survival rate is about 76%
   if at least one of the genes of the set B or of the subset B1 is expressed, the patient survival rate is about 39%,
or
   if none of the proteins of said set BP or of the subset BP1 is expressed, the patient survival rate is about 76%,
   if at least one of the proteins of the set BP or of the subset BP1 is expressed, the patient survival rate is about 39%,
or
   if none of antibodies directed against the proteins of said set BP or of the subset BP1 is expressed, the patient survival rate is about 76%,
if at least one antibody directed against at least one of the proteins of the set BP or of the subset BP1 is expressed, the patient survival rate is about 39%,
when said tumor is BAS:
   if none of the genes of the set A or of subsets A1 or A2 is expressed, the patient survival rate is about 19%
   if at least one of the genes of the set A or of subsets A1 or A2 is expressed, the patient survival rate is about 0%,
or
   if none of the proteins of the set AP or of subsets AP1 or AP2 is expressed, the patient survival rate is about 19%
   if at least one of the proteins of the set AP or of subsets AP1 or AP2 is expressed, the patient survival rate is about 0%,
or
   if none of antibodies directed against the proteins of said set AP or of subsets AP1 or AP2 is expressed, the patient survival rate is about 19%
   if at least one antibody directed against at least one of the proteins of the set AP or of subsets AP1 or AP2 is expressed, the patient survival rate is about 0%,
when said tumor is SQC:
   if none of the genes of the set C or of the subsets C1 or C2 is expressed, the patient survival rate is about 58%
   if at least one of the genes of the set C or of the subsets C1 or C2 is expressed, the patient survival rate is about 33%,
or
   if none of the proteins of the set CP or of the subsets CP1 or CP2 is expressed, the patient survival rate is about 58%
   if at least one of the proteins of the set CP or of the subsets CP1 or CP2 is expressed, the patient survival rate is about 33%,
or
   if none of antibodies directed against the proteins of said set CP or of the subsets CP1 or CP2 is expressed, the patient survival rate is about 58%
   if at least one antibody directed against at least one of the proteins of the set CP or of the subsets CP1 or CP2 is expressed, the patient survival rate is about 33%,
when said tumor is LCNE:
   if none of the genes of the set D or of the subsets D1, D2 or D3 is expressed, the patient survival rate is about 40%
   if at least one of the genes of the set D or of the subsets D1, D2 or D3 is expressed, the patient survival rate is about 0%,
or
   if none of the proteins of the set DP or of the subsets DP1, DP2 or DP3 is expressed, the patient survival rate is about 40%
   if at least one of the proteins of the set DP or of the subsets DP1, DP2 or DP3 is expressed, the patient survival rate is about 0%,
or
   if none of antibodies directed against the proteins of said set DP or of the subsets DP1, DP2 or DP3 is expressed, the patient survival rate is about 40%,
   if at least one antibody directed against at least one of the proteins of the set DP or of the subsets DP1, DP2 or DP3 is expressed, the patient survival rate is about 0%.

In one particular embodiment, the invention relates to the use as defined above, où
when said tumor is SQC:
   if none of the genes of the set C or of the subsets C1 or C2 is expressed, the patient survival rate is about 58%
   if one of the genes the set C or of the subsets C1 or C2 is expressed, the patient survival rate is about 45%,
   if at least two of the genes of the set C or of the subsets C1 or C2 is expressed, the patient survival rate is about 14%,
or
   if none of the protein of the set CP or of the subsets CP1 or CP2 is expressed, the patient survival rate is about 58%
   if one of the genes the set C or of the subsets CP1 or CP2 is expressed, the patient survival rate is about 45%,
   if at least two of the genes of the set C or of the subsets CP1 or CP2 is expressed, the patient survival rate is about 14%,
or
   if none of antibodies directed against the proteins of said set CP or of the subsets CP1 or CP2 is expressed, the patient survival rate is about 58%
   if one antibody directed against at least one of the proteins of the set CP or of the subsets CP1, CP2 or CP3, the patient survival rate is about 45%,
   if at least two antibody directed against at least one of the proteins of the set CP or of the subsets CP1, CP2 or CP3, the patient survival rate is about 14%.

In another advantageous embodiment, the invention relates to the use as defined above, wherein during a period of time of 60 months from the diagnosis of said lung cancer,

wherein
when said tumor is ADK:
   if none of the genes of said set B or of the subset B1 is expressed, the patient survival rate is about 82%
   if at least one of the genes of the set B or of the subset B1 is expressed, the patient survival rate is about 46%,
or
   if none of the proteins of said set BP or of the subset BP1 is expressed, the patient survival rate is about 82%,
   if at least one of the proteins of the set BP or of the subset BP1 is expressed, the patient survival rate is about 46%,
or
   if none of antibodies directed against the proteins of said set BP or of the subset BP1 is expressed, the patient survival rate is about 82%,
   if at least one antibody directed against at least one of the proteins of the set BP or of the subset BP1 is expressed, the patient survival rate is about 46%,
when said tumor is BAS:
   if none of the genes of the set A or of subsets A1 or A2 is expressed, the patient survival rate is about 35%
   if at least one of the genes of the set A or of subsets A1 or A2 is expressed, the patient survival rate is about 7%,
or
   if none of the proteins of the set AP or of subsets AP1 or AP2 is expressed, the patient survival rate is about 35%
   if at least one of the proteins of the set AP or of subsets AP1 or AP2 is expressed, the patient survival rate is about 7%,
or
   if none of antibodies directed against the proteins of said set AP or of subsets AP1 or AP2 is expressed, the patient survival rate is about 35%
   if at least one antibody directed against at least one of the proteins of the set AP or of subsets AP1 or AP2 is expressed, the patient survival rate is about 7%,
when said tumor is SQC:
   if none of the genes of the set C or of the subsets C1 or C2 is expressed, the patient survival rate is about 77%
   if at least one of the genes of the set C or of the subsets C1 or C2 is expressed, the patient survival rate is about 42%,
or
   if none of the proteins of the set CP or of the subsets CP1 or CP2 is expressed, the patient survival rate is about 77%
   if at least one of the proteins of the set CP or of the subsets CP1 or CP2 is expressed, the patient survival rate is about 42%,
or
   if none of antibodies directed against the proteins of said set CP or of the subsets CP1 or CP2 is expressed, the patient survival rate is about 77%
   if at least one antibody directed against at least one of the proteins of the set CP or of the subsets CP1 or CP2 is expressed, the patient survival rate is about 42%,
when said tumor is LCNE:
   if none of the genes of the set D or of the subsets D1, D2 or D3 is expressed, the patient survival rate is about 55%
   if at least one of the genes of the set D or of the subsets D1, D2 or D3 is expressed, the patient survival rate is about 4%,
or
   if none of the proteins of the set DP or of the subsets DP1, DP2 or DP3 is expressed, the patient survival rate is about 55%
   if at least one of the proteins of the set DP or of the subsets DP1, DP2 or DP3 is expressed, the patient survival rate is about 4%,
or
   if none of antibodies directed against the proteins of said set DP or of the subsets DP1, DP2 or DP3 is expressed, the patient survival rate is about 55%,
   if at least one antibody directed against at least one of the proteins of the set DP or of the subsets DP1, DP2 or DP3 is expressed, the patient survival rate is about 4%.

In one particular embodiment, the invention relates to the use as defined above, où
when said tumor is SQC:
   if none of the genes of the set C or of the subsets C1 or C2 is expressed, the patient survival rate is about 77%
   if one of the genes the set C or of the subsets C1 or C2 is expressed, the patient survival rate is about 59%,
   if at least two of the genes of the set C or of the subsets C1 or C2 is expressed, the patient survival rate is about 14%,
or
   if none of the protein of the set CP or of the subsets CP1 or CP2 is expressed, the patient survival rate is about 77%
   if one of the genes the set C or of the subsets CP1 or CP2 is expressed, the patient survival rate is about 59%,
   if at least two of the genes of the set C or of the subsets CP1 or CP2 is expressed, the patient survival rate is about 14%,
or
   if none of antibodies directed against the proteins of said set CP or of the subsets CP1 or CP2 is expressed, the patient survival rate is about 77%
   if one antibody directed against at least one of the proteins of the set CP or of the subsets CP1 or CP2, the patient survival rate is about 59%,
   if at least two antibody directed against at least one of the proteins of the set CP or of the subsets CP1 or CP2, the patient survival rate is about 14%.

In another advantageous embodiment, the invention relates to the use as defined above, wherein during a period of time of 30 months from the diagnosis of said lung cancer,

wherein
when said tumor is ADK:
   if none of the genes of said set B or of the subset B1 is expressed, the patient survival rate is about 86%
   if at least one of the genes of the set B or of the subset B1 is expressed, the patient survival rate is about 63%,
or
   if none of the proteins of said set BP or of the subset BP1 is expressed, the patient survival rate is about 86%,
   if at least one of the proteins of the set BP or of the subset BP1 is expressed, the patient survival rate is about 63%,
or
   if none of antibodies directed against the proteins of said set BP or of the subset BP1 is expressed, the patient survival rate is about 86%,
   if at least one antibody directed against at least one of the proteins of the set BP or of the subset BP1 is expressed, the patient survival rate is about 63%,
when said tumor is BAS:
   if none of the genes of the set A or of subsets A1 or A2 is expressed, the patient survival rate is about 65%
   if at least one of the genes of the set A or of subsets A1 or A2 is expressed, the patient survival rate is about 33%,
or
   if none of the proteins of the set AP or of subsets AP1 or AP2 is expressed, the patient survival rate is about 63%
   if at least one of the proteins of the set AP or of subsets AP1 or AP2 is expressed, the patient survival rate is about 33%,
or
   if none of antibodies directed against the proteins of said set AP or of subsets AP1 or AP2 is expressed, the patient survival rate is about 65%
   if at least one antibody directed against at least one of the proteins of the set AP or of subsets AP1 or AP2 is expressed, the patient survival rate is about 33%,
when said tumor is SQC:
   if none of the genes of the set C or of the subsets C1 or C2 is expressed, the patient survival rate is about 88%
   if at least one of the genes of the set C or of the subsets C1 or C2 is expressed, the patient survival rate is about 64%,
or
   if none of the proteins of the set CP or of the subsets CP1 or CP2 is expressed, the patient survival rate is about 88%
   if at least one of the proteins of the set CP or of the subsets CP1 or CP2 is expressed, the patient survival rate is about 64%,
or
   if none of antibodies directed against the proteins of said set CP or of the subsets CP1 or CP2 is expressed, the patient survival rate is about 88%
   if at least one antibody directed against at least one of the proteins of the set CP or of the subsets CP1 or CP2 is expressed, the patient survival rate is about 64%,
when said tumor is LCNE:
   if none of the genes of the set D or of the subsets D1, D2 or D3 is expressed, the patient survival rate is about 65%
   if at least one of the genes of the set D or of the subsets D1, D2 or D3 is expressed, the patient survival rate is about 7%,
or
   if none of the proteins of the set DP or of the subsets DP1, DP2 or DP3 is expressed, the patient survival rate is about 65%
   if at least one of the proteins of the set DP or of the subsets DP1, DP2 or DP3 is expressed, the patient survival rate is about 7%,
or
   if none of antibodies directed against the proteins of said set DP or of the subsets DP1, DP2 or DP3 is expressed, the patient survival rate is about 65%,
   if at least one antibody directed against at least one of the proteins of the set DP or of the subsets DP1, DP2 or DP3 is expressed, the patient survival rate is about 7%.

In one particular embodiment, the invention relates to the use as defined above, où
when said tumor is SQC:
   if none of the genes of the set C or of the subsets C1 or C2 is expressed, the patient survival rate is about 88%
if one of the genes the set C or of the subsets C1 or C2 is expressed, the patient survival rate is about 73%,
   if at least two of the genes of the set C or of the subsets C1 or C2 is expressed, the patient survival rate is about 50%,
or
   if none of the protein of the set CP or of the subsets CP1 or CP2 is expressed, the patient survival rate is about 88%
   if one of the genes the set C or of the subsets CP1 or CP2 is expressed, the patient survival rate is about 73%,
   if at least two of the genes of the set C or of the subsets CP1 or CP2 is expressed, the patient survival rate is about 50%,
or
   if none of antibodies directed against the proteins of said set CP or of the subsets CP1 or CP2 is expressed, the patient survival rate is about 88%
   if one antibody directed against at least one of the proteins of the set CP or of the subsets CP1 or CP2, the patient survival rate is about 73%,
   if at least two antibody directed against at least one of the proteins of the set CP or of the subsets CP1 or CP2, the patient survival rate is about 50%.

Advantageously, the invention relates to the use of at least one element chosen among:
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first set A of 4 genes comprising or consisting of the nucleic acid sequences SEQ ID NO: 1 to 4,
   b. at least one gene belongs to a second set B of 3 genes comprising or consisting of the nucleic acid sequences SEQ ID NO: 5 to 7
   c. at least one gene belongs to a third set C of 6 genes comprising or consisting of the nucleic acid sequences SEQ ID NO: 8 to 13, and
   d. at least one gene belongs to a fourth set D of 9 genes comprising or consisting of the nucleic acid sequences SEQ ID NO: 14-23,
at least 4 proteins being such that
   a. at least one protein belongs to a first set AP of 4 proteins comprising or consisting of the amino acid sequences SEQ ID NO: 24-27,
   b. at least one protein belongs to a second set BP of 3 proteins comprising or consisting of the amino acid sequences SEQ ID NO: 28-30, and
   c. at least one protein belongs to a third set CP of 6 proteins comprising or consisting of the amino acid sequences SEQ ID NO: 31-36,
   d. at least one protein belongs to a fourth set DP of 9 proteins comprising or consisting of the amino acid sequences SEQ ID NO: 37-46,
at least 4 antibodies directed against said 4 proteins being such that
   a. at least one antibody that interact with at least one protein that belongs to a first set AP of 4 proteins comprising or consisting of the amino acid sequences SEQ ID NO: 24-27,
   b. at least one antibody that interact with at least one protein that belongs a second set BP of 3 proteins comprising or consisting of the amino acid sequences SEQ ID NO: 28-30,
   c. at least one antibody that interact with at least one protein that belongs a third set CP of 6 proteins comprising or consisting of the amino acid sequences SEQ ID NO: 31-36, and
   d. at least one antibody that interact with at least one protein that belongs a fourth set DP of 9 proteins comprising or consisting of the amino acid sequences SEQ ID NO: 37-46,
      for the implementation of a prognosis method, preferably in vitro, of the survival rate of a patient afflicted by a lung cancer, said lung cancer being previously classified as a lung tumour belonging from the group consisting of squamous cell carcinoma (SQC), adenocarcinoma (ADK), large cell neuro-endocrine tumours (LCNE) and basaloid tumours (BAS),
      wherein
the nucleic acid sequences SEQ ID NO: 1 to 4 and/or the amino acid sequences SEQ ID NO: 24 to 27 are expressed in BAS,
the nucleic acid sequences SEQ ID NO: 5 to 7 and/or the amino acid sequences SEQ ID NO: 28 to 30 are expressed in ADK,
the nucleic acid sequences SEQ ID NO: 8 to 13 and/or the amino acid sequences SEQ ID NO: 31 to 36 are expressed in SQC,
the nucleic acid sequences SEQ ID NO: 14 to 23 and/or the amino acid sequences SEQ ID NO: 37 to 46 are expressed in LCNE,
   said prognosis being such that:
   either
during a period of time of 30 months after the diagnosis of said lung cancer
   when said tumor is ADK:
      if none of the 23 genes, or of the 23 proteins, or of the 23 antibodies of said set is expressed, the patient survival rate is about 86%, or more
      if at least one of the genes, or of the proteins, or of the antibodies of the set B is expressed, the patient survival rate is about 63%,
   when said tumor is BAS:
      if none of the 23 genes, or of the 23 proteins, or of the 23 antibodies of said set is expressed, the patient survival rate is about 65%, or more
      if at least one of the genes, or of the proteins, or of the antibodies of the set A is expressed, the patient survival rate is about 33%,
   when said tumor is SQC:
      if none of the 23 genes, or of the 23 proteins, or of the 23 antibodies of said set is expressed, the patient survival rate is about 88%, or more
      if at least one of the genes, or of the proteins, or of the antibodies of the set C is expressed, the patient survival rate is about 64%
   when said tumor is LCNE:
      if none of the 23 genes, or of the 23 proteins, or of the 23 antibodies of said set is expressed, the patient survival rate is about 65%, or more
      if at least one of the genes, or of the proteins, or of the antibodies of the set D is expressed, the patient survival rate is about 7%,
         or,
during a period of time of 60 months after the diagnosis of said lung cancer
   when said tumor is ADK:
      if none of the 23 genes, or of the 23 proteins, or of the 23 antibodies of said set is expressed, the patient survival rate is about 82%, or more
      if at least one of the genes, or of the proteins, or of the antibodies of the set B is expressed, the patient survival rate is about 46%,
   when said tumor is BAS:
      if none of the 23 genes, or of the 23 proteins, or of the 23 antibodies of said set is expressed, the patient survival rate is about 35%, or more
      if at least one of the genes, or of the proteins, or of the antibodies of the set A is expressed, the patient survival rate is about 7%,
   when said tumor is SQC:
      if none of the 23 genes, or of the 23 proteins, or of the 23 antibodies of said set is expressed, the patient survival rate is about 77%, or more
      if at least one of the genes, or of the proteins, or of the antibodies of the set C is expressed, the patient survival rate is about 42%
   when said tumor is LCNE:
      if none of the 23 genes, or of the 23 proteins, or of the 23 antibodies of said set is expressed, the patient survival rate is about 55%, or more
      if at least one of the genes, or of the proteins, or of the antibodies of the set D is expressed, the patient survival rate is about 4%,
         or,
during a period of time of 120 months after the diagnosis of said lung cancer
   when said tumor is ADK:
      if none of the 23 genes, or of the 23 proteins, or of the 23 antibodies of said set is expressed, the patient survival rate is about 76%, or more
      if at least one of the genes, or of the proteins, or of the antibodies of the set B is expressed, the patient survival rate is about 39%,
   when said tumor is BAS:
      if none of the 23 genes, or of the 23 proteins, or of the 23 antibodies of said set is expressed, the patient survival rate is about 19%, or more
      if at least one of the genes, or of the proteins, or of the antibodies of the set A is expressed, the patient survival rate is about 0%,
   when said tumor is SQC:
      if none of the 23 genes, or of the 23 proteins, or of the 23 antibodies of said set is expressed, the patient survival rate is about 58%, or more
      if at least one of the genes, or of the proteins, or of the antibodies of the set C is expressed, the patient survival rate is about 33%,
   when said tumor is LCNE:
      if none of the 23 genes, or of the 23 proteins, or of the 23 antibodies of said set is expressed, the patient survival rate is about 40%, or more
      if at least one of the genes, or of the proteins, or of the antibodies of the set D is expressed, the patient survival rate is about 0%,
         wherein said gene, protein or antibody is determined as being expressed when:
either it is expressed in a sample of a patient afflicted by a lung cancer but not in a control sample of an healthy individual,
or it is expressed above a threshold corresponding to a background signal observed in a series of reference healthy tissues for each detection method, and
   said gene, protein or antibody is determined as being not expressed when:
it is neither expressed in a sample of a patient afflicted by a lung cancer nor in a control sample of an healthy individual,
or it is expressed in a sample of a patient afflicted by a lung cancer at a level substantially equal or inferior to the level in a control sample of an healthy individual.

According to the invention “if none of the 23 genes, or of the 23 proteins, or of the 23 antibodies of said set A, B, C, D, AP, BP, CP, DP, or subranges as defined above, is expressed, the patient survival rate is about X %, or more” means that the survival rate of said patient is at the worst X % at the determined period of time (30, 60 or 120 months) but can be more favourable. Thus, when none of 23 genes, or of the 23 proteins, or of the 23 antibodies of said set A, B, C, D, AP, BP, CP, DP, or subranges as defined above, is expressed, the survival rate belong to the range [X %-100%], wherein X is the percentage as mentioned above.

However, the probability is most closer to the X % than the 100% of survival.

In another advantageous embodiment, the invention relates to the use as defined above, of:
at least 4 genes chosen among a group of 23 genes comprising or consisting of the nucleic acid sequences SEQ ID NO 1 to 23,
or fragments of said genes
or complementary sequences of said genes
or sequences having at least 80% homology with said genes or fragment thereof,
or protein coded by said genes, said proteins comprising or consisting of the nucleic acid sequences SEQ ID NO 24 to 46,
   said at least 4 genes being such that
a. at least one gene belongs to a first set A of 4 genes comprising or consisting of the nucleic acid sequences SEQ ID NO: 1 to 4,
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
   for the implementation of a prognosis method, preferably in vitro, of the survival rate of a patient afflicted by a lung cancer, said lung cancer being previously classified as a lung tumour belonging from the group consisting of ADK, SQC, BAS et LCNE,
   wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
   said prognosis being such that:
during a period of time of 30 months after the diagnosis of said lung cancer
   when said tumor is ADK:
      if none of the 23 genes of said set is expressed, the patient survival rate is about 86%, or more
      if at least one of the genes of the set B is expressed, the patient survival rate is about 63%,
   when said tumor is BAS:
      if none of the 23 genes of said set is expressed, the patient survival rate is about 65%, or more
      if at least one of the genes of the set A is expressed, the patient survival rate is about 33%,
   when said tumor is SQC:
      if none of the 23 genes of said set is expressed, the patient survival rate is about 88%, or more
      if at least one of the genes of the set C is expressed, the patient survival rate is about 64%
   when said tumor is LCNE:
      if none of the 23 genes of said set is expressed, the patient survival rate is about 65%, or more
      if at least one of the genes of the set D is expressed, the patient survival rate is about 7%,
         or,
during a period of time of 60 months after the diagnosis of said lung cancer
   when said tumor is ADK:
      if none of the 23 genes of said set is expressed, the patient survival rate is about 82%, or more
      if at least one of the genes of the set B is expressed, the patient survival rate is about 46%,
   when said tumor is BAS:
      if none of the 23 genes of said set is expressed, the patient survival rate is about 35%, or more
      if at least one of the genes of the set A is expressed, the patient survival rate is about 7%,
   when said tumor is SQC:
      if none of the 23 genes of said set is expressed, the patient survival rate is about 77%, or more
      if at least one of the genes of the set C is expressed, the patient survival rate is about 42%
   when said tumor is LCNE:
      if none of the 23 genes of said set is expressed, the patient survival rate is about 55%, or more
      if at least one of the genes of the set D is expressed, the patient survival rate is about 4%,
         or,
during a period of time of 120 months after the diagnosis of said lung cancer
   when said tumor is ADK:
      if none of the 23 genes of said set is expressed, the patient survival rate is about 76%, or more
      if at least one of the genes of the set B is expressed, the patient survival rate is about 39%,
   when said tumor is BAS:
      if none of the 23 genes of said set is expressed, the patient survival rate is about 19%, or more
      if at least one of the genes of the set A is expressed, the patient survival rate is about 0%,
   when said tumor is SQC:
      if none of the 23 genes of said set is expressed, the patient survival rate is about 58%, or more
      if at least one of the genes of the set C is expressed, the patient survival rate is about 33%,
   when said tumor is LCNE:
      if none of the 23 genes of said set is expressed, the patient survival rate is about 40%, or more
      if at least one of the genes of the set D is expressed, the patient survival rate is about 0%.

In another advantageous embodiment, the invention relates to the use as defined above,

wherein
a. said at least one gene belongs to a first set A of 4 genes comprising or consisting of the nucleic acid sequences SEQ ID NO: 1 to 4, is chosen among the nucleic acid sequence SEQ ID NO: 1 to 3, i.e belongs to a first subset A2 as defined above,
b. at least one gene belongs to a second set B of 3 genes comprising or consisting of the nucleic acid sequences SEQ ID NO: 5 to 7, is chosen among the nucleic acid sequence SEQ ID NO: 5 or 6, i.e belongs to a second subset B1 as defined above,
c. at least one gene belongs to a third set C of 6 genes comprising or consisting of the nucleic acid sequences SEQ ID NO: 8 to 13, is chosen among the nucleic acid sequence SEQ ID NO: 8 to 11, i.e belongs to a third subset C2 as defined above and
d. at least one gene belongs to a fourth set D of 9 genes comprising or consisting of the nucleic acid sequences SEQ ID NO: 14-23, is chosen among the nucleic acid sequence SEQ ID NO: 16 to 20 i.e belongs to a fourth subset D5 as defined above.

In another advantageous embodiment, the invention to the use as defined above, wherein
during a period of time of 30 months after the diagnosis of said lung cancer
   when said tumor is SQC:
      if none of the 23 genes of said set is expressed, the patient survival rate is about 88%
      if one of the genes of the set C is expressed, the patient survival rate is about 73%,
      if at least two of the genes of the set C are expressed, the patient survival rate is about 50%.
during a period of time of 60 months after the diagnosis of said lung cancer
   when said tumor is SQC:
      if none of the 23 genes of said set is expressed, the patient survival rate is about 77%
      if one of the genes of the set C is expressed, the patient survival rate is about 59%,
      if at least two of the genes of the set C are expressed, the patient survival rate is about 14%,
during a period of time of 120 months after the diagnosis of said lung cancer
   when said tumor is SQC:
      if none of the 23 genes of said set is expressed, the patient survival rate is about 58%
      if one of the genes of the set C is expressed, the patient survival rate is about 45%,
      if at least two of the genes of the set C are expressed, the patient survival rate is about 14%.

The invention also relates to a prognosis method, preferably in vitro, of the survival rate of a patient afflicted by a lung tumour, from a biological sample containing said lung tumor, at a time from 30 to 120 months after the diagnosis of said lung cancer, as defined above, said lung cancer being previously classified as a lung tumour belonging from the group consisting of ADK, SQC, BAS et LCNE,

said method comprising a step of measuring, in said biological sample, the expression of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
   said
at least 4 genes being such that
   a. at least one gene belongs to a first set A of 4 genes comprising or consisting of the nucleic acid sequences SEQ ID NO: 1 to 4, or a subset A1 as defined above,
   b. at least one gene belongs to a second set B of 3 genes comprising or consisting of the nucleic acid sequences SEQ ID NO: 5 to 7, or a subset B1 or B2 as defined above
   c. at least one gene belongs to a third set C of 6 genes comprising or consisting of the nucleic acid sequences SEQ ID NO: 8 to 13, or a subset C1 or C2 as defined above, and
   d. at least one gene belongs to a fourth set D of 9 genes comprising or consisting of the nucleic acid sequences SEQ ID NO: 14-23, or a subset D1, D2 or D3 as defined above,
at least 4 proteins being such that
   a. at least one protein belongs to a first set AP of 4 proteins comprising or consisting of the amino acid sequences SEQ ID NO: 24-27, or a subset AP1 as defined above
   b. at least one protein belongs to a second set BP of 3 proteins comprising or consisting of the amino acid sequences SEQ ID NO: 28-30, or a subset BP1 or BP2 as defined above
   c. at least one protein belongs to a second set CP of 6 proteins comprising or consisting of the amino acid sequences SEQ ID NO: 31-36, or a subset CP1 or CP2 as defined above,
   d. at least one protein belongs to a second set DP of 9 proteins comprising or consisting of the amino acid sequences SEQ ID NO: 37-46, or a subset DP1, DP2 or DP3 as defined above,
at least 4 antibodies directed against said 4 proteins being such that
   a. at least one antibody that ineract with at least one protein that belongs to a first set AP of 4 proteins comprising or consisting of the amino acid sequences SEQ ID NO: 24-27, or a subset AP1 as defined above
   b. at least one antibody that ineract with at least one protein that belongs a second set BP of 3 proteins comprising or consisting of the amino acid sequences SEQ ID NO: 28-30, or a subset BP1 or BP2 as defined above
   c. at least one antibody that ineract with at least one protein that belongs a third set CP of 6 proteins comprising or consisting of the amino acid sequences SEQ ID NO: 31-36, or a subset CP1 or CP2 as defined above,
   d. at least one antibody that ineract with at least one protein that belongs a fourth set DP of 9 proteins comprising or consisting of the amino acid sequences SEQ ID NO: 37-46, or a subset DP1, DP2 or DP3 as defined above,
      wherein
the nucleic acid sequences SEQ ID NO: 1 to 4 and/or the amino acid sequences SEQ ID NO: 24 to 27 are expressed in BAS,
the nucleic acid sequences SEQ ID NO: 5 to 7 and/or the amino acid sequences SEQ ID NO: 28 to 30 are expressed in ADK,
the nucleic acid sequences SEQ ID NO: 8 to 13 and/or the amino acid sequences SEQ ID NO: 31 to 36 are expressed in SQC,
the nucleic acid sequences SEQ ID NO: 14 to 23 and/or the amino acid sequences SEQ ID NO: 37 to 46 are expressed in LCNE,
   said prognosis being such that:
   either
if none of the genes of said set A, B, C and D is expressed, the patient survival rate during a period of time from 30 to 120 months after the diagnosis of said lung cancer is from about 59% to about 78% or more, and
if at least one gene of at least one set A, B, C or D is expressed, the patient survival rate during a period of time from 30 to 120 months after the diagnosis of said lung cancer is about from about 3% to about 70%,
   or
if none of the proteins of said set AP, BP, CP and DP is expressed, the patient survival rate during a period of time from 30 to 120 months after the diagnosis of said lung cancer is from about 59% to about 78% or more, and
if at least one protein of at least one set AP, BP, CP and DP is expressed, the patient survival rate during a period of time from 30 to 120 months after the diagnosis of said lung cancer is about from about 3% to about 70%,
   or
if none of the antibodies directed against the proteins of said set AP, BP, CP and DP is expressed, the patient survival rate during a period of time from 30 to 120 months after the diagnosis of said lung cancer is from about 59% to about 78% or more, and
if at least one antibody directed against one protein of said set AP, BP, CP and DP is expressed, the patient survival rate during a period of time from 30 to 120 months after the diagnosis of said lung cancer is about from about 3% to about 70%.

The invention also relates to a prognosis method, preferably in vitro, of the survival rate of a patient afflicted by a lung tumour, from a biological sample containing said lung tumor, at a time from 30 to 120 months after the diagnosis of said lung cancer, as defined above,

said method comprising a step of measuring, in said biological sample, the expression of
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
   said
at least 4 genes being such that
   a. at least one gene belongs to a first set A of 4 genes comprising or consisting of the nucleic acid sequences SEQ ID NO: 1 to 4, or a subset A1 as defined above,
   b. at least one gene belongs to a second set B of 3 genes comprising or consisting of the nucleic acid sequences SEQ ID NO: 5 to 7, or a subset B1 or B2 as defined above
   c. at least one gene belongs to a third set C of 6 genes comprising or consisting of the nucleic acid sequences SEQ ID NO: 8 to 13, or a subset C1 or C2 as defined above, and
   d. at least one gene belongs to a fourth set D of 9 genes comprising or consisting of the nucleic acid sequences SEQ ID NO: 14-23, or a subset D1, D2 or D3 as defined above,
      wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
   said prognosis being such that:
if none of the genes of said set A, B, C and D is expressed, the patient survival rate during a period of time from 30 to 120 months after the diagnosis of said lung cancer is from about 59% to about 78% or more, and
if at least one gene of at least one set A, B, C or D is expressed, the patient survival rate during a period of time from 30 to 120 months after the diagnosis of said lung cancer is about from about 3% to about 70%.

Advantageously, the invention relates to a prognosis method, preferably in vitro, of the survival rate of a patient afflicted by a lung tumour, from a biological sample containing said lung tumor belonging from the group consisting of ADK, SQC, BAS et LCNE, at a time from 30 to 120 months after the diagnosis of said lung cancer,

said method comprising a step of measuring, in said biological sample, the expression of
at least 4 genes chosen among a group of 23 genes comprising or consisting of the nucleic acid sequences SEQ ID NO 1 to 23,
or fragments of said genes
or complementary sequences of said genes
   said at least 4 genes being such that
a. at least one gene belongs to a first set A of 4 genes comprising or consisting of the nucleic acid sequences SEQ ID NO: 1 to 4
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
   wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
   said prognosis being such that:
during a period of time of 30 months after the diagnosis of said lung cancer
   when said tumor is ADK:
      if none of the 23 genes of said set is expressed, the patient survival rate is about 86%, or more
      if at least one of the genes of the set B is expressed, the patient survival rate is about 63%,
   when said tumor is BAS:
      if none of the 23 genes of said set is expressed, the patient survival rate is about 65%, or more
      if at least one of the genes of the set A is expressed, the patient survival rate is about 33%,
   when said tumor is SQC:
      if none of the 23 genes of said set is expressed, the patient survival rate is about 88%, or more
      if at least one of the genes of the set C is expressed, the patient survival rate is about 64%
   when said tumor is LCNE:
      if none of the 23 genes of said set is expressed, the patient survival rate is about 65%, or more
      if at least one of the genes of the set D is expressed, the patient survival rate is about 7%,
         or,
during a period of time of 60 months after the diagnosis of said lung cancer
   when said tumor is ADK:
      if none of the 23 genes of said set is expressed, the patient survival rate is about 82%, or more
      if at least one of the genes of the set B is expressed, the patient survival rate is about 46%,
   when said tumor is BAS:
      if none of the 23 genes of said set is expressed, the patient survival rate is about 35%, or more
      if at least one of the genes of the set A is expressed, the patient survival rate is about 7%,
   when said tumor is SQC:
      if none of the 23 genes of said set is expressed, the patient survival rate is about 77%, or more
      if at least one of the genes of the set C is expressed, the patient survival rate is about 42%
   when said tumor is LCNE:
      if none of the 23 genes of said set is expressed, the patient survival rate is about 55%, or more
      if at least one of the genes of the set D is expressed, the patient survival rate is about 4%,
         or,
during a period of time of 120 months after the diagnosis of said lung cancer
   when said tumor is ADK:
      if none of the 23 genes of said set is expressed, the patient survival rate is about 76%, or more
      if at least one of the genes of the set B is expressed, the patient survival rate is about 39%,
   when said tumor is BAS:
      if none of the 23 genes of said set is expressed, the patient survival rate is about 19%, or more
      if at least one of the genes of the set A is expressed, the patient survival rate is about 0%,
   when said tumor is SQC:
      if none of the 23 genes of said set is expressed, the patient survival rate is about 58%, or more
      if at least one of the genes of the set C is expressed, the patient survival rate is about 33%,
   when said tumor is LCNE:
      if none of the 23 genes of said set is expressed, the patient survival rate is about 40%, or more
      if at least one of the genes of the set D is expressed, the patient survival rate is about 0%.

In one advantageous embodiment, the invention relates to the prognosis method as defined above, wherein
a. said at least one gene belongs to a first set A of 4 genes comprising or consisting of the nucleic acid sequences SEQ ID NO: 1 to 4, is chosen among the nucleic acid sequence SEQ ID NO: 1 to 3,
b. at least one gene belongs to a second set B of 3 genes comprising or consisting of the nucleic acid sequences SEQ ID NO: 5 to 7, is chosen among the nucleic acid sequence SEQ ID NO: 5 or 6,
c. at least one gene belongs to a third set C of 6 genes comprising or consisting of the nucleic acid sequences SEQ ID NO: 8 to 13, is chosen among the nucleic acid sequence SEQ ID NO: 8 to 11, and
d. at least one gene belongs to a fourth set D of 9 genes comprising or consisting of the nucleic acid sequences SEQ ID NO: 14-23, is chosen among the nucleic acid sequence SEQ ID NO: 16 to 20.

In one another advantageous embodiment, the invention relates to the prognosis method previously defined, wherein
during a period of time of 30 months after the diagnosis of said lung cancer
   when said tumor is SQC:
      if none of the 23 genes of said set is expressed, the patient survival rate is about 88%
      if one of the genes of the set C is expressed, the patient survival rate is about 73%,
      if at least two of the genes of the set C are expressed, the patient survival rate is about 50%.
during a period of time of 60 months after the diagnosis of said lung cancer
   when said tumor is SQC:
      if none of the 23 genes of said set is expressed, the patient survival rate is about 77%
      if one of the genes of the set C is expressed, the patient survival rate is about 59%,
      if at least two of the genes of the set C are expressed, the patient survival rate is about 14%,
during a period of time of 120 months after the diagnosis of said lung cancer
   when said tumor is SQC:
      if none of the 23 genes of said set is expressed, the patient survival rate is about 58%
      if one of the genes of the set C is expressed, the patient survival rate is about 45%,
      if at least two of the genes of the set C are expressed, the patient survival rate is about 14%.

In another advantageous embodiment, the invention relates to a prognosis method previously defined, wherein the step of measuring is carried out by using a technique chosen among the set consisting of:
a. Quantitative PCR,
b. DNA CHIP, and
c. Northern blot.

In another advantageous embodiment, the invention relates to a prognosis method as previously defined, wherein the step of measuring is carried out by using nucleic acid molecules consisting of from 15 to 100 nucleotides molecules being complementary to said at least 4 genes.

In one advantageous embodiment, the invention relates to a prognosis method as defined above, wherein the step of measuring is carried out by DNA CHIP using
at least one nucleic acid probe comprising or being constituted by the nucleic acid sequence SEQ ID NO: 47 to SEQ ID NO: 50,
at least one nucleic acid probe comprising or being constituted by the nucleic acid sequence SEQ ID NO: 51 to SEQ ID NO: 53,
at least one nucleic acid probe comprising or being constituted by the nucleic acid sequence SEQ ID NO: 54 to SEQ ID NO: 59, and
at least one nucleic acid probe comprising or being constituted by the nucleic acid sequence SEQ ID NO: 60 to SEQ ID NO: 69.

In another advantageous embodiment, the invention relates to the method as defined above, wherein the step of measuring is carried out by DNA CHIP using at least 2, preferably at least 3 nucleic acid probe as defined above.

An advantageous embodiment of the invention relates to the above method wherein the nucleic acid probes comprising or being constituted by the nucleic acid sequences SEQ ID NO: 47 to 69 are used, together.

In the invention the correspondence between the genes and the nucleic acid probes are as follows: SEQ ID NO:1 is able to be detected by the nucleic acid probe comprising or being constituted by the nucleic acid sequence SEQ ID NO: 47, SEQ ID NO:2 is able to be detected by the nucleic acid probe comprising or being constituted by the nucleic acid sequence SEQ ID NO: 48, SEQ ID NO:3 is able to be detected by the nucleic acid probe comprising or being constituted by the nucleic acid sequence SEQ ID NO: 49, SEQ ID NO:4 is able to be detected by the nucleic acid probe comprising or being constituted by the nucleic acid sequence SEQ ID NO: 50, SEQ ID NO:5 is able to be detected by the nucleic acid probe comprising or being constituted by the nucleic acid sequence SEQ ID NO: 51, SEQ ID NO:6 is able to be detected by the nucleic acid probe comprising or being constituted by the nucleic acid sequence SEQ ID NO: 52, SEQ ID NO:7 is able to be detected by the nucleic acid probe comprising or being constituted by the nucleic acid sequence SEQ ID NO: 53, SEQ ID NO:8 is able to be detected by the nucleic acid probe comprising or being constituted by the nucleic acid sequence SEQ ID NO: 54, SEQ ID NO:9 is able to be detected by the nucleic acid probe comprising or being constituted by the nucleic acid sequence SEQ ID NO: 55, SEQ ID NO:10 is able to be detected by the nucleic acid probe comprising or being constituted by the nucleic acid sequence SEQ ID NO: 56, SEQ ID NO:11 is able to be detected by the nucleic acid probe comprising or being constituted by the nucleic acid sequence SEQ ID NO: 57, SEQ ID NO:12 is able to be detected by the nucleic acid probe comprising or being constituted by the nucleic acid sequence SEQ ID NO: 58, SEQ ID NO:13 is able to be detected by the nucleic acid probe comprising or being constituted by the nucleic acid sequence SEQ ID NO: 59, SEQ ID NO:14 is able to be detected by the nucleic acid probe comprising or being constituted by the nucleic acid sequence SEQ ID NO: 60, SEQ ID NO:15 is able to be detected by the nucleic acid probe comprising or being constituted by the nucleic acid sequence SEQ ID NO: 61, SEQ ID NO:16 is able to be detected by the nucleic acid probe comprising or being constituted by the nucleic acid sequence SEQ ID NO: 62, SEQ ID NO:17 is able to be detected by the nucleic acid probe comprising or being constituted by the nucleic acid sequence SEQ ID NO: 63, SEQ ID NO:18 is able to be detected by the nucleic acid probe comprising or being constituted by the nucleic acid sequence SEQ ID NO: 64, SEQ ID NO:19 is able to be detected by the nucleic acid probe comprising or being constituted by the nucleic acid sequence SEQ ID NO: 65, SEQ ID NO:20 is able to be detected by the nucleic acid probe comprising or being constituted by the nucleic acid sequence SEQ ID NO: 66, SEQ ID NO:21 is able to be detected by the nucleic acid probe comprising or being constituted by the nucleic acid sequence SEQ ID NO: 67, SEQ ID NO:22 is able to be detected by the nucleic acid probe comprising or being constituted by the nucleic acid sequence SEQ ID NO: 68, SEQ ID NO:23 is able to be detected by the nucleic acid probe comprising or being constituted by the nucleic acid sequence SEQ ID NO: 69.

The invention also relates to a prognosis method, preferably in vitro, of the survival rate of a patient afflicted by a lung tumour, from a biological sample containing said lung tumor, at a time from 30 to 120 months after the diagnosis of said lung cancer,

said method comprising a step of measuring, in said biological sample, the expression of
at least 4 proteins chosen among a set of 23 proteins coded by 23 genes comprising or consisting of the nucleic acid sequences SEQ ID NO 1 to 23,
or fragments of said proteins,
   said at least 4 proteins being such that
at least 4 proteins being such that
   a. at least one protein belongs to a first set AP of 4 proteins comprising or consisting of the amino acid sequences SEQ ID NO: 24-27,
   b. at least one protein belongs to a second set BP of 3 proteins comprising or consisting of the amino acid sequences SEQ ID NO: 28-30,
   c. at least one protein belongs to a third set CP of 6 proteins comprising or consisting of the amino acid sequences SEQ ID NO: 31-36,
   d. at least one protein belongs to a fourth set DP of 9 proteins comprising or consisting of the amino acid sequences SEQ ID NO: 37-46,
      wherein
the amino acid sequences SEQ ID NO: 24 to 27 are expressed in BAS,
the amino acid sequences SEQ ID NO: 28 to 30 are expressed in ADK,
the amino acid sequences SEQ ID NO: 31 to 36 are expressed in SQC,
the amino acid sequences SEQ ID NO: 37 to 46 are expressed in LCNE,
   said prognosis method being such that
if none of the proteins is expressed, the patient survival rate is from about 59% to about 78%, or more, and
if at least one protein of said set AP, BP, CP and DP is expressed, the patient survival rate from about 3% to about 70%.

In an advantageous embodiment the invention relates a prognosis method, preferably in vitro, of the survival rate of a patient afflicted by a lung tumour, from a biological sample containing said lung tumor, at a time from 30 to 120 months after the diagnosis of said lung cancer,

said method comprising a step of measuring, in said biological sample, the expression of
at least 4 proteins chosen among a set of 23 proteins coded by 23 genes comprising or consisting of the nucleic acid sequences SEQ ID NO 1 to 23,
or fragments of said proteins,
   said at least 4 proteins being such that
at least 4 proteins being such that
   a. at least one protein belongs to a first set AP of 4 proteins comprising or consisting of the amino acid sequences SEQ ID NO: 24-27,
   b. at least one protein belongs to a second set BP of 3 proteins comprising or consisting of the amino acid sequences SEQ ID NO: 28-30,
   c. at least one protein belongs to a third set CP of 6 proteins comprising or consisting of the amino acid sequences SEQ ID NO: 31-36,
   d. at least one protein belongs to a fourth set DP of 9 proteins comprising or consisting of the amino acid sequences SEQ ID NO: 37-46,
      wherein
the amino acid sequences SEQ ID NO: 24 to 27 are expressed in BAS,
the amino acid sequences SEQ ID NO: 28 to 30 are expressed in ADK,
the amino acid sequences SEQ ID NO: 31 to 36 are expressed in SQC,
the amino acid sequences SEQ ID NO: 37 to 46 are expressed in LCNE,
   said prognosis method being such that
during a period of time of 30 months after the diagnosis of said lung cancer
   when said tumor is ADK:
      if none of the 23 proteins of said set is expressed, the patient survival rate is about 86%, or more
      if at least one of the proteins of the set BP is expressed, the patient survival rate is about 63%,
   when said tumor is BAS:
      if none of the 23 proteins of said set is expressed, the patient survival rate is about 65%, or more
      if at least one of the proteins of the set AP is expressed, the patient survival rate is about 33%,
   when said tumor is SQC:
      if none of the 23 proteins of said set is expressed, the patient survival rate is about 88%, or more
      if at least one of the proteins of the set CP is expressed, the patient survival rate is about 64%
   when said tumor is LCNE:
      if none of the 23 proteins of said set is expressed, the patient survival rate is about 65%, or more
      if at least one of the proteins of the set DP is expressed, the patient survival rate is about 7%,
         or,
during a period of time of 60 months after the diagnosis of said lung cancer
   when said tumor is ADK:
      if none of the 23 proteins of said set is expressed, the patient survival rate is about 82%, or more
      if at least one of the proteins of the set BP is expressed, the patient survival rate is about 46%,
   when said tumor is BAS:
      if none of the 23 proteins of said set is expressed, the patient survival rate is about 35%, or more
      if at least one of the proteins of the set AP is expressed, the patient survival rate is about 7%,
   when said tumor is SQC:
      if none of the 23 proteins of said set is expressed, the patient survival rate is about 77%, or more
      if at least one of the proteins of the set CP is expressed, the patient survival rate is about 42%
   when said tumor is LCNE:
      if none of the 23 proteins of said set is expressed, the patient survival rate is about 55%, or more
      if at least one of the proteins of the set DP is expressed, the patient survival rate is about 4%,
         or,
during a period of time of 120 months after the diagnosis of said lung cancer
   when said tumor is ADK:
      if none of the 23 proteins of said set is expressed, the patient survival rate is about 76%, or more
      if at least one of the proteins of the set BP is expressed, the patient survival rate is about 39%,
   when said tumor is BAS:
      if none of the 23 proteins of said set is expressed, the patient survival rate is about 19%, or more
      if at least one of the proteins of the set AP is expressed, the patient survival rate is about 0%,
   when said tumor is SQC:
      if none of the 23 proteins of said set is expressed, the patient survival rate is about 58%, or more
      if at least one of the proteins of the set CP is expressed, the patient survival rate is about 33%,
   when said tumor is LCNE:
      if none of the 23 proteins of said set is expressed, the patient survival rate is about 40%, or more
      if at least one of the proteins of the set DP is expressed, the patient survival rate is about 0%.

In one advantageous embodiment, the invention relates to the prognosis method, as defined above, wherein
a. said at least one gene belongs to a first set AP of 4 genes comprising or consisting of the nucleic acid sequences SEQ ID NO: 24 to 27, is chosen among the nucleic acid sequence SEQ ID NO: 24 to 27,
b. at least one gene belongs to a second set BP of 3 genes comprising or consisting of the nucleic acid sequences SEQ ID NO: 28 to 30, is chosen among the nucleic acid sequence SEQ ID NO: 28 or 29,
c. at least one gene belongs to a third set CP of 6 genes comprising or consisting of the nucleic acid sequences SEQ ID NO: 31 to 36, is chosen among the nucleic acid sequence SEQ ID NO: 31 to 34, and
d. at least one gene belongs to a fourth set DP of 9 genes comprising or consisting of the nucleic acid sequences SEQ ID NO: 37 to 46, is chosen among the nucleic acid sequence SEQ ID NO: 37 to 43.

In another advantageous embodiment, the invention relates to the prognosis method as defined above, wherein
during a period of time of 30 months after the diagnosis of said lung cancer
   when said tumor is SQC:
      if none of the 23 proteins of said set is expressed, the patient survival rate is about 88%
      if one of the proteins of the set CP is expressed, the patient survival rate is about 73%,
      if at least two of the proteins of the set CP are expressed, the patient survival rate is about 50%.
during a period of time of 60 months after the diagnosis of said lung cancer
   when said tumor is SQC:
      if none of the 23 proteins of said set is expressed, the patient survival rate is about 77%
      if one of the proteins of the set CP is expressed, the patient survival rate is about 59%,
      if at least two of the proteins of the set CP are expressed, the patient survival rate is about 14%,
during a period of time of 120 months after the diagnosis of said lung cancer
   when said tumor is SQC:
      if none of the 23 proteins of said set is expressed, the patient survival rate is about 58%
      if one of the proteins of the set CP is expressed, the patient survival rate is about 45%,
      if at least two of the proteins of the set CP are expressed, the patient survival rate is about 14%.

In one advantageous embodiment, the invention relates to the prognosis method as defined above wherein the step of measuring is carried out by using a technique chosen among the set consisting of:
a. western Blot,
b. ELISA,
c. Immunofluorescence, and
d. Immunohistochemistry.

In one other advantageous embodiment, the invention relates to a prognosis method, as defined above, wherein the step of measuring is carried out by using antibodies directed against said at least 4 proteins coded by said at least two genes.

Another advantageous embodiment of the invention relates to a prognosis method, as defined above, further comprising a step of comparison of said measured expression to the expression in at least one control sample.

The invention also concern a kit comprising a DNA CHIP comprising at least the nucleic acid probes comprising or being constituted by the nucleic acid sequences SEQ ID NO: 47 to 69.

### FIGURES

FIG. 1 represents the cumulative survival rate (Kaplan Mayer curve) over 60 months of patients expressing (A) or not (B) the gene SEQ ID NO: 4 (gene 391). Y-axis represents cumulative survival in %, X-axis represents time in months.

FIG. 2 represents the cumulative survival rate (Kaplan Mayer curve) over 60 months of patients expressing (A) or not (B) the gene SEQ ID NO: 3 (gene 417). Y-axis represents cumulative survival in %, X-axis represents time in months.

FIG. 3 represents the cumulative survival rate (Kaplan Mayer curve) over 60 months of patients expressing (A) or not (B) the gene SEQ ID NO: 2 (gene 1261). Y-axis represents cumulative survival in %, X-axis represents time in months.

FIG. 4 represents the cumulative survival rate (Kaplan Mayer curve) over 60 months of patients expressing (A) or not (B) the following genes: SEQ ID NO: 1 (gene 1292). Y-axis represents cumulative survival in %, X-axis represents time in months.

FIG. 5 represents the cumulative survival rate (Kaplan Mayer curve) over 60 months of patients expressing (A) or not (B) at least one of the following genes: SEQ ID NO: 1 (gene 1292), SEQ ID NO: 2 (gene 1261), SEQ ID NO: 3 (gene 417) and SEQ ID NO: 4 (gene 391). Y-axis represents cumulative survival in %, X-axis represents time in months.

FIG. 6 represents the cumulative survival rate (Kaplan Mayer curve) over 60 months of patients expressing (A) or not (B) the following genes: SEQ ID NO: 7 (gene 59). Y-axis represents cumulative survival in %, X-axis represents time in months.

FIG. 7 represents the cumulative survival rate (Kaplan Mayer curve) over 60 months of patients expressing (A) or not (B) the following genes: SEQ ID NO: 6 (gene 222). Y-axis represents cumulative survival in %, X-axis represents time in months.

FIG. 8 represents the cumulative survival rate (Kaplan Mayer curve) over 60 months of patients expressing (A) or not (B) the following genes: SEQ ID NO: 5 (gene 1371). Y-axis represents cumulative survival in %, X-axis represents time in months.

FIG. 9 represents the cumulative survival rate (Kaplan Mayer curve) over 60 months of patients expressing (A) or not (B) at least one of the following genes: SEQ ID NO: 5 (gene 1371), SEQ ID NO: 6 (gene 222) and SEQ ID NO: 7 (gene 59). Y-axis represents cumulative survival in %, X-axis represents time in months.

FIG. 10 represents the cumulative survival rate (Kaplan Mayer curve) over 60 months of patients expressing (A) or not (B) the gene SEQ ID NO: 12 (gene 440). Y-axis represents cumulative survival in %, X-axis represents time in months.

FIG. 11 represents the cumulative survival rate (Kaplan Mayer curve) over 60 months of patients expressing (A) or not (B) the gene SEQ ID NO: 9 (gene 442). Y-axis represents cumulative survival in %, X-axis represents time in months.

FIG. 12 represents the cumulative survival rate (Kaplan Mayer curve) over 60 months of patients expressing (A) or not (B) the gene SEQ ID NO: 8 (gene 266). Y-axis represents cumulative survival in %, X-axis represents time in months.

FIG. 13 represents the cumulative survival rate (Kaplan Mayer curve) over 60 months of patients expressing (A) or not (B) the gene SEQ ID NO: 13 (gene 1161). Y-axis represents cumulative survival in %, X-axis represents time in months.

FIG. 14 represents the cumulative survival rate (Kaplan Mayer curve) over 60 months of patients expressing (A) or not (B) the gene SEQ ID NO: 11 (gene 356). Y-axis represents cumulative survival in %, X-axis represents time in months.

FIG. 15 represents the cumulative survival rate (Kaplan Mayer curve) over 60 months of patients expressing (A) or not (B) the gene SEQ ID NO: 10 (gene 160). Y-axis represents cumulative survival in %, X-axis represents time in months.

FIG. 16 represents the cumulative survival rate (Kaplan Mayer curve) over 60 months of patients expressing (A) or not (B) at least one of the following genes: SEQ ID NO: 8 (gene 266), SEQ ID NO: 9 (gene 442), SEQ ID NO: 10 (gene 160), SEQ ID NO: 11 (gene 356), SEQ ID NO: 12 (gene 440) and SEQ ID NO: 13 (gene 1161). Y-axis represents cumulative survival in %, X-axis represents time in months.

FIG. 17 represents the cumulative survival rate (Kaplan Mayer curve) over 60 months of patients expressing one (B), or two or more (A) or no (C) genes chosen among SEQ ID NO: 8 (gene 266), SEQ ID NO: 9 (gene 442), SEQ ID NO: 10 (gene 160), SEQ ID NO: 11 (gene 356), SEQ ID NO: 12 (gene 440) and SEQ ID NO: 13 (gene 1161). Y-axis represents cumulative survival in %, X-axis represents time in months.

FIG. 18 represents the cumulative survival rate (Kaplan Mayer curve) over 60 months of patients expressing (A) or not (B) the gene SEQ ID NO: 22 (gene 117). Y-axis represents cumulative survival in %, X-axis represents time in months.

FIG. 19 represents the cumulative survival rate (Kaplan Mayer curve) over 60 months of patients expressing (A) or not (B) the gene SEQ ID NO: 21 (gene 118). Y-axis represents cumulative survival in %, X-axis represents time in months.

FIG. 20 represents the cumulative survival rate (Kaplan Mayer curve) over 60 months of patients expressing (A) or not (B) the gene SEQ ID NO: 18 (gene 144). Y-axis represents cumulative survival in %, X-axis represents time in months.

FIG. 21 represents the cumulative survival rate (Kaplan Mayer curve) over 60 months of patients expressing (A) or not (B) the gene SEQ ID NO: 16 (gene 424). Y-axis represents cumulative survival in %, X-axis represents time in months.

FIG. 22 represents the cumulative survival rate (Kaplan Mayer curve) over 60 months of patients expressing (A) or not (B) the gene SEQ ID NO: 19 (gene 437). Y-axis represents cumulative survival in %, X-axis represents time in months.

FIG. 23 represents the cumulative survival rate (Kaplan Mayer curve) over 60 months of patients expressing (A) or not (B) the gene SEQ ID NO: 17 (gene 125). Y-axis represents cumulative survival in %, X-axis represents time in months.

FIG. 24 represents the cumulative survival rate (Kaplan Mayer curve) over 60 months of patients expressing (A) or not (B) the gene SEQ ID NO: 20 (gene 766). Y-axis represents cumulative survival in %, X-axis represents time in months.

FIG. 25 represents the cumulative survival rate (Kaplan Mayer curve) over 60 months of patients expressing (A) or not (B) the gene SEQ ID NO: 14 (gene 71). Y-axis represents cumulative survival in %, X-axis represents time in months.

FIG. 26 represents the cumulative survival rate (Kaplan Mayer curve) over 60 months of patients expressing (A) or not (B) the gene SEQ ID NO: 23 (gene 390). Y-axis represents cumulative survival in %, X-axis represents time in months.

FIG. 27 represents the cumulative survival rate (Kaplan Mayer curve) over 60 months of patients expressing (A) or not (B) the gene SEQ ID NO: 15 (gene 1165). Y-axis represents cumulative survival in %, X-axis represents time in months.

FIG. 28 represents the cumulative survival rate (Kaplan Mayer curve) over 60 months of patients expressing (A) or not (B) at least one of the following genes: SEQ ID NO: 16 (gene 424), SEQ ID NO: 18 (gene 144), SEQ ID NO: 21 (gene 118) and SEQ ID NO: 22 (gene 117). Y-axis represents cumulative survival in %, X-axis represents time in months.

FIG. 29 represents the cumulative survival rate (Kaplan Mayer curve) over 60 months of patients expressing (A) or not (B) at least one of the following genes: SEQ ID NO: 16 (gene 424), SEQ ID NO: 17 (gene 125), SEQ ID NO: 18 (gene 144), SEQ ID NO: 21 (gene 118) and SEQ ID NO: 22 (gene 117). Y-axis represents cumulative survival in %, X-axis represents time in months.

FIG. 30 represents the cumulative survival rate (Kaplan Mayer curve) over 60 months of patients expressing one (B), or two or more (A) or no (C) genes chosen among SEQ ID NO: 16 (gene 424), SEQ ID NO: 17 (gene 125), SEQ ID NO: 18 (gene 144), SEQ ID NO: 21 (gene 118) and SEQ ID NO: 22 (gene 117). Y-axis represents cumulative survival in %, X-axis represents time in months.

FIG. 31 represents the cumulative survival rate (Kaplan Mayer curve) over 60 months of patients expressing (A) or not (B) at least one of the following genes: SEQ ID NO: 14 (gene 71), SEQ ID NO: 16 (gene 424), SEQ ID NO: 17 (gene 125), SEQ ID NO: 18 (gene 144), SEQ ID NO: 21 (gene 118) and SEQ ID NO: 22 (gene 117). Y-axis represents cumulative survival in %, X-axis represents time in months.

FIG. 32 represents the cumulative survival rate (Kaplan Mayer curve) over 60 months of patients expressing one (B), or two or more (A) or no (C) genes chosen among SEQ ID NO: 14 (gene 71), SEQ ID NO: 16 (gene 424), SEQ ID NO: 17 (gene 125), SEQ ID NO: 18 (gene 144), SEQ ID NO: 21 (gene 118) and SEQ ID NO: 22 (gene 117). Y-axis represents cumulative survival in %, X-axis represents time in months.

### EXAMPLE

This work is based on a group of 23 genes, which can be used to establish the survival prognosis of lung tumour patients. All these genes are actively repressed and silent in normal adult somatic cells, since their expression in strictly restricted to placenta or male germinal cells. The inventors have demonstrated that the aberrant expression in malignant cells of at least one of these genes is associated with significantly poorer prognosis for lung cancer patients. Moreover, the detection of the expression of several combinations of these genes allows predicting prognosis in lung tumour patients with higher significance and accuracy than with individual genes.

I—Methodological Approach

The following procedure allowed identifying the genes mentioned above.

### Overview

The expression of the 497 testis- and placenta-specific genes was studied in a series of 271 lung cancer samples by extracting the corresponding expression data from genome-wide transcriptomic data (the latter were obtained by C. and E. Brambilla supported by the Ligue CIT program (Carte d'Identite des Tumeurs)).

For each gene, the patients were divided into two groups,
those expressing the gene,
and those not expressing the gene.

For each of the 497 genes of the list, the global and disease free survival probabilities were compared between the patients expressing the gene and those not expressing the gene.

This was done considering the whole period of the study, as well as for 10 years, 5 years and 2.5 years of follow-up.

This was performed considering patients of the main histological subtypes of this population (ADK=adenocarcinoma; BAS=basaloid; LCNE=Large cell neuroendocrine; SQC=squamous cell tumour).

The genes discriminating the patients with good or bad prognosis with a significance corresponding to a p value=<0.05 (Logrank p value, obtained when comparing cumulative global survival and/or disease free survival over 5 years between patients expressing or not expressing the gene) were selected as candidate prognosis markers.

For all these genes, the correlation between their expression and prognosis was validated in at least one of the two following lung published cancer transcriptomic studies with survival clinical data using the same Affymetrix technology (website GEO: http://www.ncbi.nlm.nih.gov/geo, respectively GSE4576 and GSE8894).

These studies were selected as external populations of lung cancer patients in order to validate the survival data obtained by analysing the transcriptomic data of the Brambilla study.

### Detailed Procedure

1—Establishment of a List of Placenta and Testis Restricted Genes and Analysis of their Aberrant Expression in Lung Cancer Patients
1a—A list of 497 human genes whose expression was restricted to placenta or male germ cells was established as defined in WO2009/121878. These genes are never expressed in normal adult somatic tissues (adult somatic tissues comprise all tissues except germinal cells, foetal tissues and placenta).
1b—Expression data were extracted from a series of 112 normal adult somatic tissues randomly selected from a genome wide study of normal human tissues (GSE3526 on GEO, this study was chosen because it uses the same probes and measurement technology to detect gene expression as the Brambilla study: Affymetrix Human Genome U133 Plus 2.0 Array). The CEL files (raw data) from the control samples were downloaded from GEO. They were entered in the Genespring software and normalized (RMA algorithm) simultaneously with the CEL files from the Brambilla study.
1c—For each of the 497 genes/probes, the mean hybridization intensity signal value of the 112 control samples and 2 standard deviation was defined as the threshold for expression. This threshold was used to distinguish between the cancer samples expressing the gene and those not expressing the gene.

The measurement of expression of the genes using Affymetrix microarrays involves the hybridization of fluorescence labeled cDNAs from each tissue sample on microarrays containing gene-specific probes, the fluorescence intensity signal corresponding to each probe of the microarray is measured and changed into a raw value. The absolute value of the fluorescence intensity signal is highly variable and probe-dependent (different probes corresponding to the same gene can give different intensities of fluorescence). Therefore, on the basis of these absolute fluorescence intensity values it is generally not possible to determine whether a gene is expressed or not, and commonly people use this technique to assess variations of expression between samples.

The definition of a precise threshold for expression was possible because the selected 497 genes are NOT expressed in any normal adult somatic tissue. Therefore the signal values obtained in the 112 normal adult somatic control samples give a high confidence set of values corresponding to the background noise signal, which allow further analyses.

A threshold signal value for expression could not have been defined for genes, which do not have a restricted expression pattern.

Indeed in all these types of transcriptomic experiments the background noise signal value is highly dependent on the sequence of the probe. For instance several probes representative of the same gene generally give different signal values. In the case of non-restricted genes (most genes have a pattern of expression, which is not restricted to germinal cells or placenta), it is therefore impossible to use these signal values as “absolute” indicators of the presence or absence of expression. However, one can compare expression levels between two groups of tissues.

Therefore, in this particular study, since the Inventors have previously demonstrated that all the studied 497 genes are NOT expressed in normal adult somatic tissues, the Inventors were able to define a threshold differentiating expression (ON) and non-expression (OFF). This is a specific key feature of their approach.

1d—Based on this threshold, the expression of each of the 497 genes in each of the samples was defined as negative (OFF) or positive (ON) as follow.

In each cancer sample:
if the normalised signal value was above this threshold, the gene was considered as aberrantly expressed in this sample (gene ON),
if it was under this threshold, it was considered as not expressed (gene OFF).
   1e—From the Brambilla study (271 cases of lung cancer), the Inventors found that 130 of the 497 genes were aberrantly expressed in at least 1% of these lung cancer cases.
   2—Correlation Between the Expression of Each Individual Gene (of the List of 130 Genes) and the Prognosis for Survival in the Lung Cancer Patients, and Selection of 23 Genes Individually Associated with the Prognosis of a Particular Histological Subtype (These Subtypes being Either ADK or BAS or LCNE or SQC).
   2a—As a first step, using each of the 130 genes individually, we compared the global survival over a period of five years between the groups of patients expressing the gene (yes) versus those not expressing the gene (no). A Logrank Mantel-Cox test was performed and a p value was calculated. This analysis was performed with each one of the following populations: ADK cases (n=91), BAS cases (n=46), LCNE cases (n=47), SQC cases (n=62).
   2b—A total of 23 genes were selected, whose individual expression was significantly associated with a poorer prognosis (as measured by the cumulative global survival and/or disease-free survival over five years; p<0.05) in the Brambilla lung cancer study, as well as in at least one of the external validation populations (see table 1). The following table 1 lists the 23 genes associated with poor prognosis of specific histological subtypes of lung cancer, confirmed by external studies.

Gene ID corresponds to the nomenclature of the Inventors, Type corresponds to the histological subtype, SEQ ID NO gene correspond to the nucleic acid sequence according to the invention, SEQ ID NO prot corresponds to the corresponding amino acid sequence, Gene name corresponds to the name of the genes, ADK_, SQC_ BAS_ and LCNE_ corresponds to the Logrank p value for each histological type: i.e. significance of difference in cumulative global survival probabilities over 5 years between patients expressing the gene and those not expressing the gene, nb of ADK_, SQC_ BAS_ and LCNE_ corresponds to the number of patients expressing the genes.

TABLE 1

| | | SEQ | SEQ | | | nb | | nb | | nb | | nb |
|---|---|---|---|---|---|---|---|---|---|---|---|---|
| Gene | | ID NO | ID NO | | | ADK+ | | BAS+ | | LCNE+ | | SQC+ |
| ID | type | gene | prot | Gene Name | ADK | (/91) | BAS | (/46) | LCNE | (/41) | SQC | (/62) |
| 1292 | BAS | 1 | 24 | KRTAP21-1 | | | 0.068 | 7 | | | | |
| 1261 | BAS | 2 | 25 | C12orf37 | | | 0.033 | 4 | | | | |
| 417 | BAS | 3 | 26 | WBSCR28 | | | 0.021 | 3 | | | | |
| 391 | BAS | 4 | 27 | RBM46 | | | 0.017 | 7 | | | | |
| 1371 | ADK | 5 | 28 | DKFZp761D1918 | 0.033 | 10 | | | | | | |
| 222 | ADK | 6 | 29 | NBPF4 | 0.007 | 3 | | | | | | |
| 59 | ADK | 7 | 30 | ADAM12 | 0.007 | 38 | | | | | | |
| 266 | SQC | 8 | 31 | CDNA clone | | | | | | | 0.01 | 9 |
| | | | | IMAGE: 5299000 | | | | | | | | |
| 442 | SQC | 9 | 32 | SOX30 | | | | | | | 0.009 | 9 |
| 160 | SQC | 10 | 33 | CDNA clone | | | | | | | 0.048 | 25 |
| | | | | IMAGE: 5296886 | | | | | | | | |
| 356 | SQC | 11 | 34 | ASZ1 | | | | | | | 0.021 | 4 |
| 440 | SQC | 12 | 35 | SLC2A14 | | | | | | | <0.0001 | 4 |
| 1161 | SQC | 13 | 36 | COX8C | | | | | | | 0.017 | 7 |
| 71 | LCNE | 14 | 37 | TKTL2 | | | | | 0.052 | 3 | | |
| 1165 | LCNE | 15 | 38 | TPTEps1 | | | | | 0.07 | 3 | | |
| 424 | LCNE | 16 | 39 | C9orf11 | | | | | 0.002 | 19 | | |
| 125 | LCNE | 17 | 40 | PIWIL1 | | | | | 0.02 | 6 | | |
| 144 | LCNE | 18 | 41 | C19orf41 | | | | | 0.0003 | 3 | | |
| 437 | LCNE | 19 | 42 | RNF17 | | | | | 0.003 | 5 | | |
| 766 | LCNE | 20 | 43 | GALNTL5 | | | | | 0.028 | 4 | | |
| 118 | LCNE | 21 | 44 | KISS1 | | | | | <0.0001 | 3 | | |
| 117 | LCNE | 22 | 45 | IGFBP1 | | | | | <0.0001 | 5 | | |
| 390 | LCNE | 23 | 46 | MAGEB6 | | | | | 0.068 | 16 | | |

2c—A detailed quantitative evaluation of the prognosis is given in table 2A-D, using each of the 23 genes associated with poor prognosis in all lung cancer histological types. The corresponding Kaplan Meyer survival curves obtained using each of these genes are represented in figures.

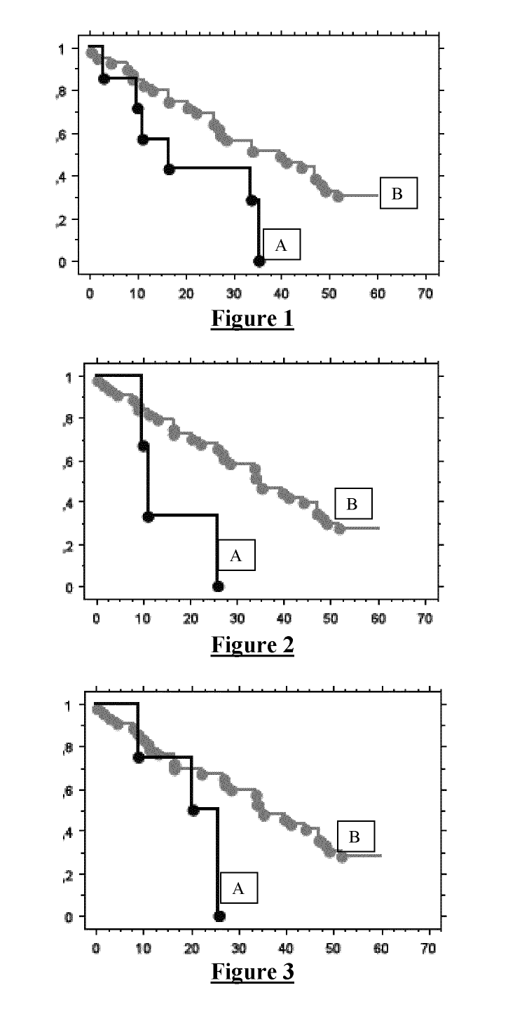

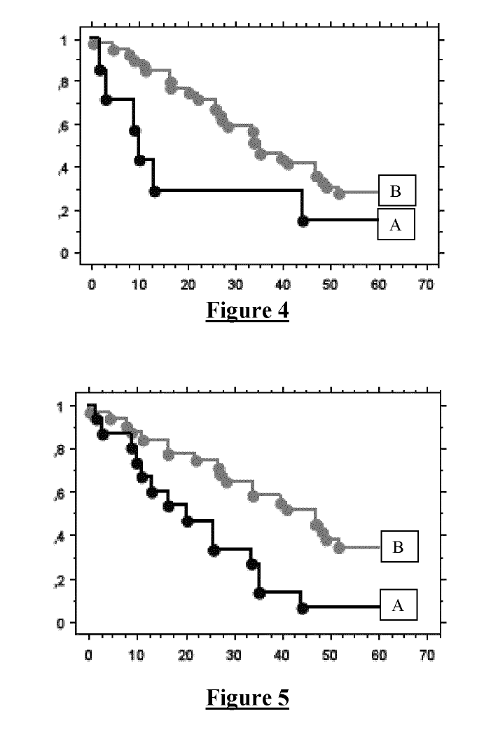

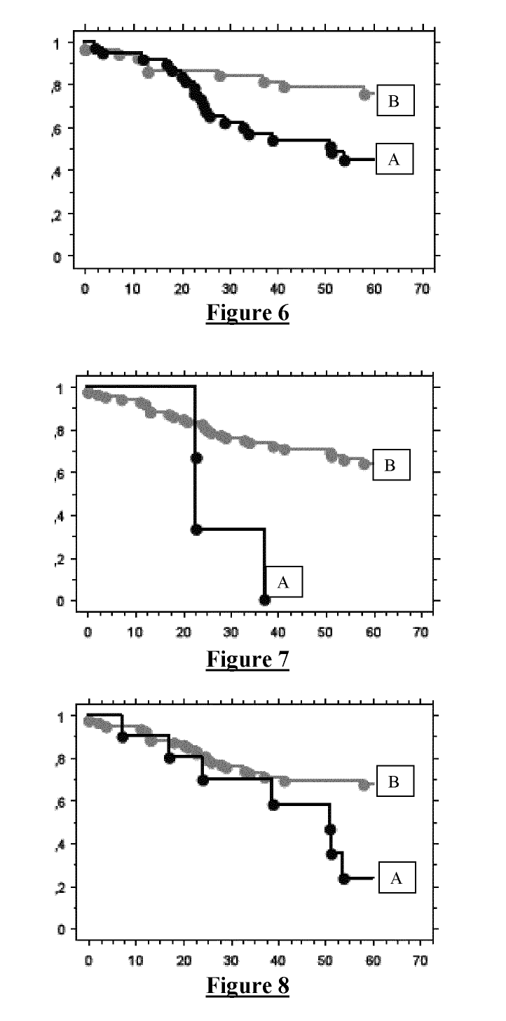

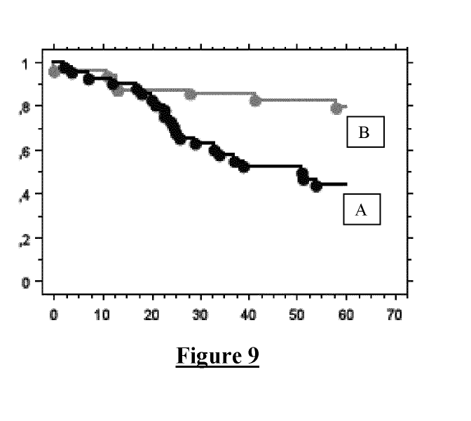

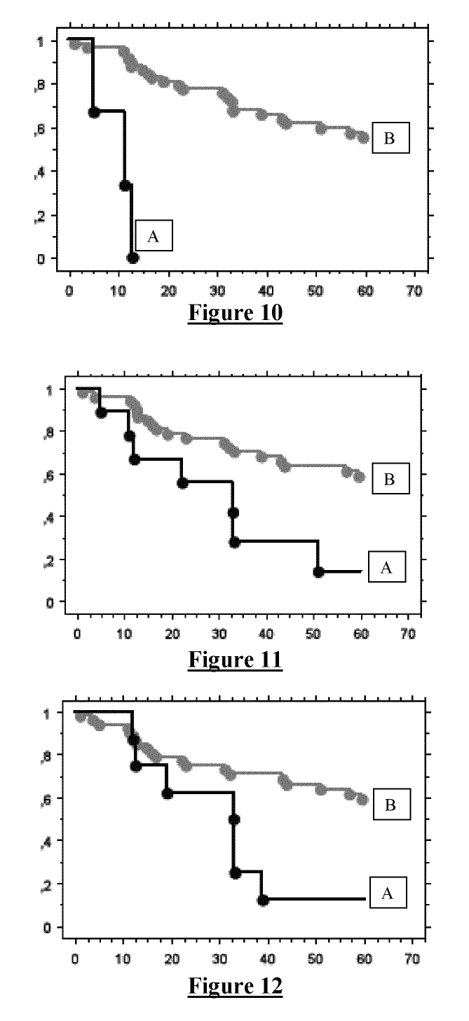

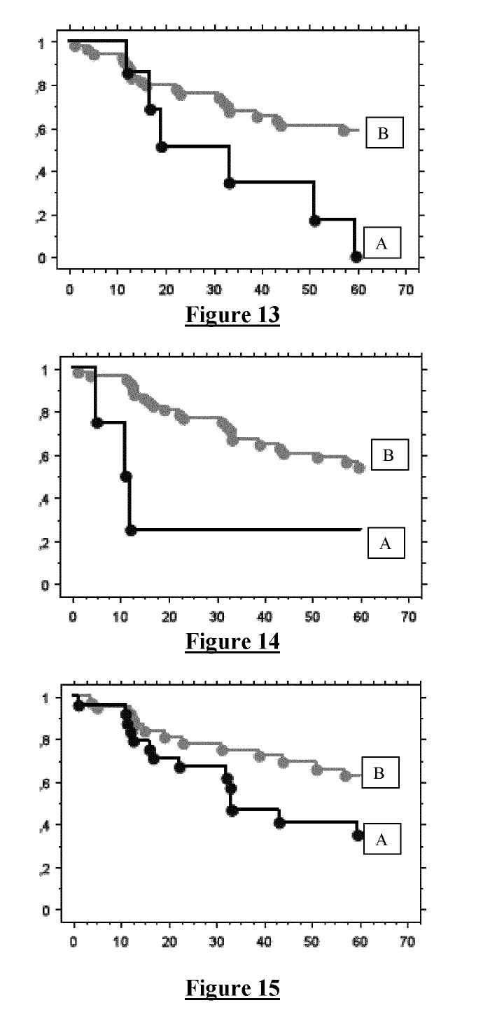

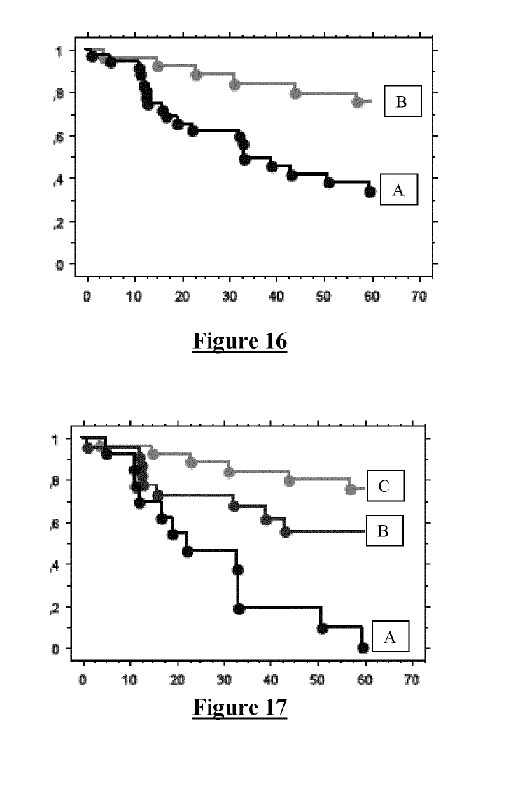

TABLE 2a

| | SEQ | | Total | Nb alive | % alive | Nb alive | % alive | Nb alive | % alive | p-value | Corresponding |
|---|---|---|---|---|---|---|---|---|---|---|---|
| Genes | ID NO | Classes | BAS | 30 m | 30 m | 60 m | 60 m | 120 m | 120 m | (Logrank Test) | figure |
| 391 | 4 | 0 | 39 | 22 | 56 | 12 | 31 | 6 | 15 | 0.017 | FIG. 1 |
| | | 1 | 7 | 3 | 43 | 0 | 0 | 0 | 0 | | |
| 417 | 3 | 0 | 43 | 25 | 58 | 12 | 28 | 6 | 14 | 0.021 | FIG. 2 |
| | | 1 | 3 | 0 | 0 | 0 | 0 | 0 | 0 | | |
| 1261 | 2 | 0 | 42 | 25 | 60 | 12 | 29 | 6 | 14 | 0.033 | FIG. 3 |
| | | 1 | 4 | 0 | 0 | 0 | 0 | 0 | 0 | | |
| 1292 | 1 | 0 | 39 | 23 | 59 | 11 | 28 | 6 | 15 | 0.068 | FIG. 4 |
| | | 1 | 7 | 2 | 29 | 1 | 14 | 0 | 0 | | |
| 391 + | 1 + | 0 | 31 | 20 | 65 | 11 | 35 | 6 | 19 | 0.003 | FIG. 5 |
| 417 + | 2 + | >=1 | 15 | 5 | 33 | 1 | 7 | 0 | 0 | | |
| 1261 + | 3 + | | | | | | | | | | |
| 1292 | 4 | | | | | | | | | | |

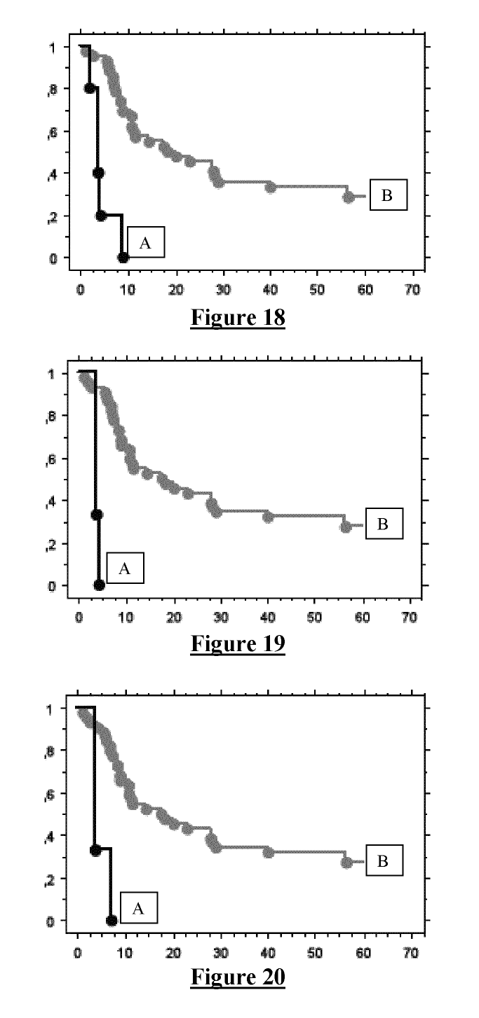

TABLE 2b

| | SEQ | | Total | Nb alive | % alive | Nb alive | % alive | Nb alive | % alive | p-value | Corresponding |
|---|---|---|---|---|---|---|---|---|---|---|---|
| Genes | ID NO | Classes | ADK | 30 m | 30 m | 60 m | 60 m | 120 m | 120 m | (Logrank Test) | figure |
| 59 | 7 | 0 | 53 | 45 | 85 | 42 | 79 | 39 | 74 | 0.007 | FIG. 6 |
| | | 1 | 38 | 24 | 63 | 18 | 47 | 15 | 39 | | |
| 222 | 6 | 0 | 88 | 68 | 77 | 60 | 68 | 54 | 61 | 0.007 | FIG. 7 |
| | | 1 | 3 | 1 | 33 | 0 | 0 | 0 | 0 | | |
| 1371 | 5 | 0 | 81 | 62 | 77 | 57 | 70 | 52 | 64 | 0.033 | FIG. 8 |
| | | 1 | 10 | 7 | 70 | 3 | 30 | 2 | 20 | | |
| 59 + | 7 + | 0 | 50 | 43 | 86 | 41 | 82 | 38 | 76 | 0.002 | FIG. 9 |
| 222 + | 6 + | >=1 | 41 | 26 | 63 | 19 | 46 | 16 | 39 | | |
| 1371 | 5 | | | | | | | | | | |

TABLE 2c

| | SEQ | | Total | Nb alive | % alive | Nb alive | % alive | Nb alive | % alive | p-value | Correspondin g |
|---|---|---|---|---|---|---|---|---|---|---|---|
| Genes | ID NO | Classes | SQC | 30 m | 30 m | 60 n | 60 m | 120 m | 120 m | (Logrank Test) | figure |
| 440 | 12 | 0 | 58 | 45 | 78 | 34 | 59 | 26 | 45 | <0.0001 | FIG. 10 |
| | | 1 | 4 | 1 | 25 | 1 | 25 | 1 | 25 | | |
| 442 | 9 | 0 | 53 | 41 | 77 | 33 | 62 | 25 | 47 | 0.009 | FIG. 11 |
| | | 1 | 9 | 5 | 56 | 2 | 22 | 2 | 22 | | |
| 266 | 8 | 0 | 53 | 40 | 75 | 33 | 62 | 25 | 47 | 0.01 | FIG. 12 |
| | | 1 | 9 | 6 | 67 | 2 | 22 | 2 | 22 | | |
| 1161 | 13 | 0 | 55 | 42 | 76 | 34 | 62 | 26 | 47 | 0.017 | FIG. 13 |
| | | 1 | 7 | 4 | 57 | 1 | 14 | 1 | 14 | | |
| 356 | 11 | 0 | 58 | 45 | 78 | 34 | 59 | 27 | 47 | 0.021 | FIG. 14 |
| | | 1 | 4 | 1 | 25 | 1 | 25 | 0 | 0 | | |
| 160 | 10 | 0 | 37 | 29 | 78 | 24 | 65 | 18 | 49 | 0.048 | FIG. 15 |
| | | 1 | 25 | 17 | 68 | 11 | 44 | 9 | 36 | | |
| 440 + | 12 + | 0 | 26 | 23 | 88 | 20 | 77 | 15 | 58 | 0.002 | FIG. 16 |
| 442 + | 9 + | | | | | | | | | | |
| 266 + | 8 + | | | | | | | | | | |
| 1161 + | 13 + | >=1 | 36 | 23 | 64 | 15 | 42 | 12 | 33 | | |
| 356 + | 11 + | | | | | | | | | | |
| 160 | 10 | | | | | | | | | | |
| 440 + | 12 + | 0 | 26 | 23 | 88 | 20 | 77 | 15 | 58 | <0.0001 | FIG. 17 |
| 442 + | 9 + | | | | | | | | | | |
| 266 + | 8 + | | | | | | | | | | |
| 1161 + | 13 + | 1 | 22 | 16 | 73 | 13 | 59 | 10 | 45 | | |
| 356 + | 11 + | | | | | | | | | | |
| 160 | 10 | >=2 | 14 | 7 | 50 | 2 | 14 | 2 | 14 | | |

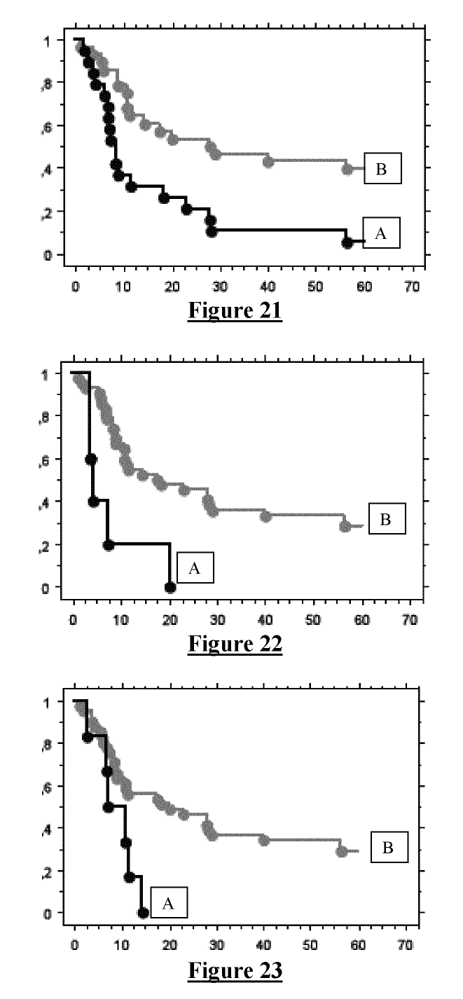

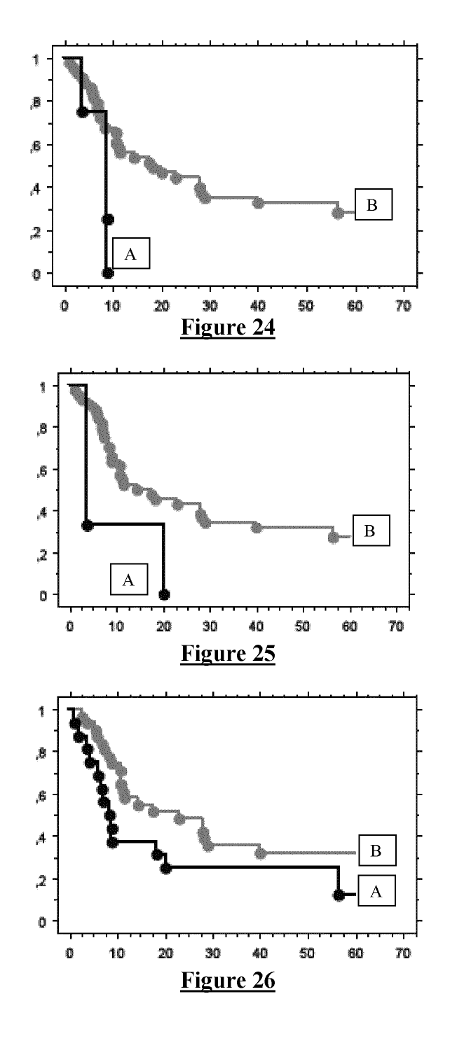

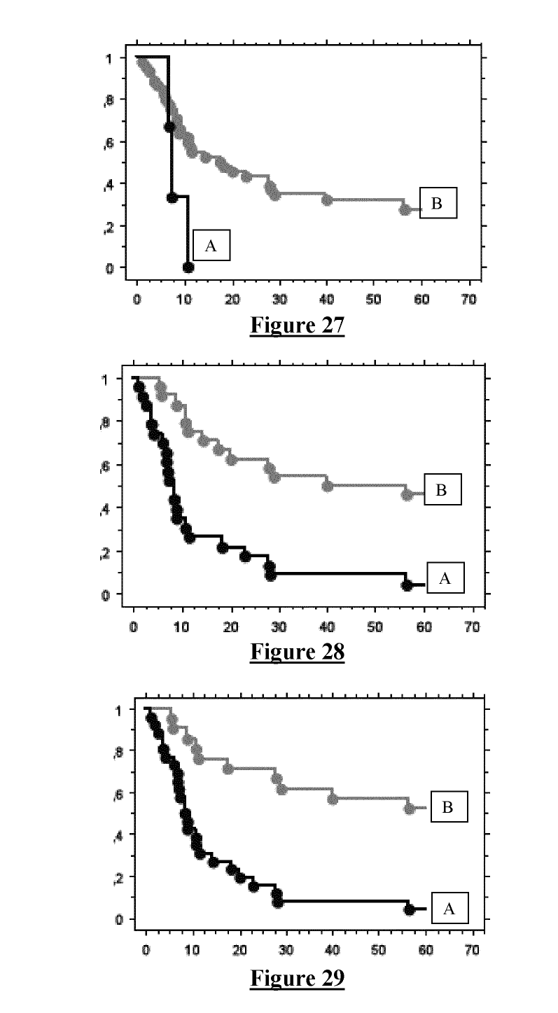

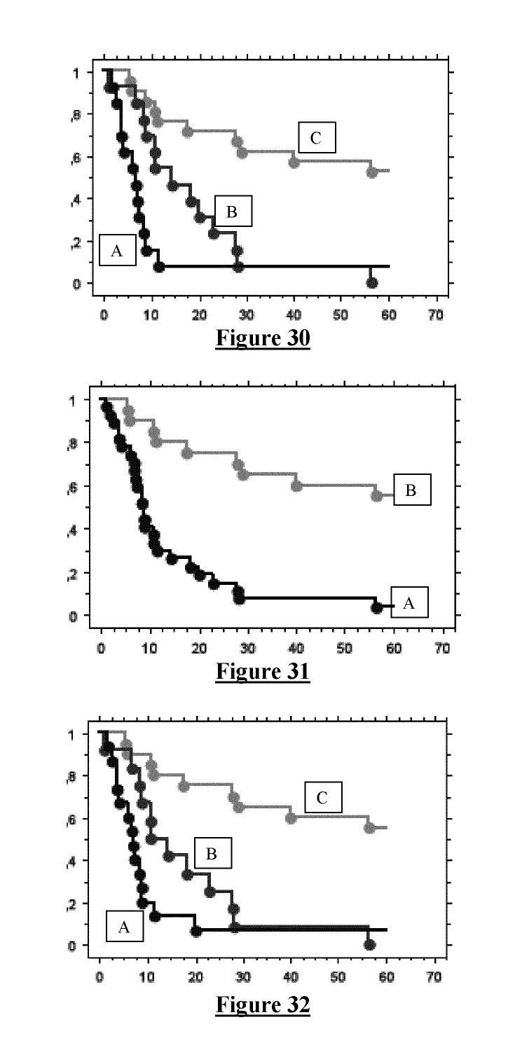

TABLE 2d

| | SEQ | | Total | Nb alive | % alive | Nb alive | % alive | Nb alive | % alive | p-value | Corresponding |
|---|---|---|---|---|---|---|---|---|---|---|---|
| Genes | ID NO | Classes | LCNE | 30 m | 30 m | 60 m | 60 m | 120 m | 120 m | (Logrank Test) | figure |
| 117 | 22 | 0 | 42 | 15 | 36 | 12 | 29 | 8 | 19 | <0.0001 | FIG. 18 |
| | | 1 | 5 | 0 | 0 | 0 | 0 | 0 | 0 | | |
| 118 | 21 | 0 | 44 | 15 | 34 | 12 | 27 | 8 | 18 | <0.0001 | FIG. 19 |
| | | 1 | 3 | 0 | 0 | 0 | 0 | 0 | 0 | | |
| 144 | 18 | 0 | 44 | 15 | 34 | 12 | 27 | 8 | 18 | 0.0003 | FIG. 20 |
| | | 1 | 3 | 0 | 0 | 0 | 0 | 0 | 0 | | |
| 424 | 16 | 0 | 28 | 13 | 46 | 11 | 39 | 8 | 29 | 0.002 | FIG. 21 |
| | | 1 | 19 | 2 | 11 | 1 | 5 | 0 | 0 | | |
| 437 | 19 | 0 | 42 | 15 | 36 | 12 | 29 | 8 | 19 | 0.003 | FIG. 22 |
| | | 1 | 5 | 0 | 0 | 0 | 0 | 0 | 0 | | |
| 125 | 17 | 0 | 41 | 15 | 37 | 12 | 29 | 8 | 20 | 0.02 | FIG. 23 |
| | | 1 | 6 | 0 | 0 | 0 | 0 | 0 | 0 | | |
| 766 | 20 | 0 | 43 | 15 | 35 | 12 | 28 | 8 | 19 | 0.028 | FIG. 24 |
| | | 1 | 4 | 0 | 0 | 0 | 0 | 0 | 0 | | |
| 71 | 14 | 0 | 44 | 15 | 34 | 12 | 27 | 8 | 18 | 0.052 | FIG. 25 |
| | | 1 | 3 | 0 | 0 | 0 | 0 | 0 | 0 | | |
| 390 | 23 | 0 | 31 | 11 | 35 | 10 | 32 | 7 | 23 | 0.068 | FIG. 26 |
| | | 1 | 16 | 4 | 25 | 2 | 13 | 1 | 6 | | |
| 1165 | 15 | 0 | 44 | 15 | 34 | 12 | 27 | 8 | 18 | 0.07 | FIG. 27 |
| | | 1 | 3 | 0 | 0 | 0 | 0 | 0 | 0 | | |
| 117 + | 22 + | 0 | 24 | 13 | 54 | 11 | 46 | 8 | 33 | <0.0001 | FIG. 28 |
| 118 + | 21 + | >=1 | 23 | 2 | 9 | 1 | 4 | 0 | 0 | | |
| 144 + | 18 + | | | | | | | | | | |
| 424 | 16 | | | | | | | | | | |
| 117 + | 22 + | 0 | 21 | 13 | 62 | 11 | 52 | 8 | 38 | <0.0001 | FIG. 29 |
| 118 + | 21 + | >=1 | 26 | 2 | 8 | 1 | 4 | 0 | 0 | | |
| 144 + | 18 + | | | | | | | | | | |
| 424 + | 16 + | | | | | | | | | | |
| 437 + | 19 + | | | | | | | | | | |
| 125 | 17 | | | | | | | | | | |
| 117 + | 22 + | 0 | 21 | 13 | 62 | 11 | 52 | 8 | 38 | <0.0001 | FIG. 30 |
| 118 + | 21 + | 1 | 13 | 1 | 8 | 0 | 0 | 0 | 0 | | |
| 144 + | 18 + | >=2 | 13 | 1 | 8 | 1 | 8 | 0 | 0 | | |
| 424 + | 16 + | | | | | | | | | | |
| 437 + | 19 + | | | | | | | | | | |
| 125 | 17 | | | | | | | | | | |
| 117 + | 22 + | 0 | 20 | 13 | 65 | 11 | 55 | 8 | 40 | <0.0001 | FIG. 31 |
| 118 + | 21 + | >=1 | 27 | 2 | 7 | 1 | 4 | 0 | 0 | | |
| 144 + | 18 + | | | | | | | | | | |
| 424 + | 16 + | | | | | | | | | | |
| 437 + | 19 + | | | | | | | | | | |
| 125 + | 17 + | | | | | | | | | | |
| 766 + | 20 + | | | | | | | | | | |
| 71 | 14 | | | | | | | | | | |
| 117 + | 22 + | 0 | 20 | 13 | 65 | 11 | 55 | 8 | 40 | <0.0001 | FIG. 32 |
| 118 + | 21 + | 1 | 12 | 1 | 8 | 0 | 0 | 0 | 0 | | |
| 144 + | 18 + | >=2 | 15 | 1 | 7 | 1 | 7 | 0 | 0 | | |
| 424 + | 16 + | | | | | | | | | | |
| 437 + | 19 + | | | | | | | | | | |
| 125 + | 17 + | | | | | | | | | | |
| 766 + | 20 + | | | | | | | | | | |
| 71 | 14 | | | | | | | | | | |

Legend of tables 2 A-D
Gene represents the inventors nomenclature of genes
SEQ ID NO corresponds to the nucleic acid molecules according to the invention
Classes correspond to Lung cancer patients not expressing (=0) or expressing (=1) the gene. In the case of combinations of several genes, some patients could express only one gene or at least one gene of the combination (=1), only 2 genes or 2 genes or more of the combination (=2) etc. . . .
Total BAS/ADK/SQC or LCNE corresponds to the number of patients of this class (considering the whole Brambilla study with 271 cases, including all histological types)
% or nb alive 30, 60, 120 month corresponds to percentage or number of patients of this class alive after 30, 60, 120 month
p-value corresponds to the significance of the difference in cumulative global survival probabilities between the different classes of patients over 2.5, 5 and 10 years (Logrank Test, considering survival curves of all the classes of patients).
Corresponding figures corresponds to the Kaplan Meyer curves.
4—Twenty Three Genes (Allow an Accurate Prognosis in Lung Cancer Patients with Specific Histological Subtypes, Either Individually or in Association
4a—Three genes, individually or in association, allow defining groups of different prognosis among ADK patients (table 2B).
4b—Four genes, individually or in association, allow defining groups of different prognosis among BAS patients (table 2A).
4c—Eleven genes, individually or in association, allow defining groups of different prognosis among LCNE patients (table 2C).
4d—Six genes, individually or in association, allow defining groups of different prognosis among SQC patients (table 2D).

### CONCLUSIONS

Using 23 genes aberrantly expressed in lung cancer, the Inventors are able to assess prognosis four lung cancer patients diagnosed with one of the four main histological subtypes of non-small cell lung cancer, including adenocarcinoma, squamous cell carcinoma, large cell neuro endocrine and basaloid tumours. These 23 genes therefore provide a basis for developing easy to use diagnosis tests for histologically classified lung tumours, in order to assess the survival probability of lung cancer patients independently from other prognosis parameters (histology, tumour size and stage . . . ) and adapt therapeutic strategies accordingly.

1. An element chosen among:
at least 4 genes chosen among a set of 23 genes comprising or consisting of the nucleic acid sequences SEQ ID NO 1 to 23,
or fragments of said least 4 genes chosen among a set of 23 genes comprising or consisting of the nucleic acid sequences SEQ ID NO 1 to 23,
or complementary sequences of said least 4 genes chosen among a set of 23 genes comprising or consisting of the nucleic acid sequences SEQ ID NO 1 to 23,
or sequences having at least 80% homology with said genes or fragment thereof,
or proteins coded by said least 4 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
said
at least 4 genes being such that
a. at least one gene belongs to a first set A of 4 genes comprising or consisting of the nucleic acid sequences SEQ ID NO: 1 to 4,
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
at least 4 proteins being such that
a. at least one protein belongs to a first set AP of 4 proteins comprising or consisting of the amino acid sequences SEQ ID NO: 24-27,
b. at least one protein belongs to a second set BP of 3 proteins comprising or consisting of the amino acid sequences SEQ ID NO: 28-30, and
c. at least one protein belongs to a third set CP of 6 proteins comprising or consisting of the amino acid sequences SEQ ID NO: 31-36,
d. at least one protein belongs to a fourth set DP of 9 proteins comprising or consisting of the amino acid sequences SEQ ID NO: 37-46,
at least 4 antibodies directed against said 4 proteins being such that
a. at least one antibody that interact with at least one protein that belongs to a first set AP of 4 proteins comprising or consisting of the amino acid sequences SEQ ID NO: 24-27,
b. at least one antibody that interact with at least one protein that belongs a second set BP of 3 proteins comprising or consisting of the amino acid sequences SEQ ID NO: 28-30,
c. at least one antibody that interact with at least one protein that belongs a third set CP of 6 proteins comprising or consisting of the amino acid sequences SEQ ID NO: 31-36, and
d. at least one antibody that interact with at least one protein that belongs a fourth set DP of 9 proteins comprising or consisting of the amino acid sequences SEQ ID NO: 37-46,
wherein said element is suitable for the implementation of a prognosis method, preferably in vitro, of the survival rate of a patient afflicted by a lung cancer, said lung cancer being previously classified as a lung tumour belonging from the group consisting of squamous cell carcinoma (SQC), adenocarcinoma (ADK), large cell neuro-endocrine tumours (LCNE) and basaloid tumours (BAS),
wherein
the nucleic acid sequences SEQ ID NO: 1 to 4 and/or the amino acid sequences SEQ ID NO: 24 to 27 are expressed in BAS,
the nucleic acid sequences SEQ ID NO: 5 to 7 and/or the amino acid sequences SEQ ID NO: 28 to 30 are expressed in ADK,
the nucleic acid sequences SEQ ID NO: 8 to 13 and/or the amino acid sequences SEQ ID NO: 31 to 36 are expressed in SQC,
the nucleic acid sequences SEQ ID NO: 14 to 23 and/or the amino acid sequences SEQ ID NO: 37 to 46 are expressed in LCNE,
said prognosis being such that:
either
during a period of time of 30 months after the diagnosis of said lung cancer
when said tumor is ADK:
if none of the 23 genes, or of the 23 proteins, or of the 23 antibodies of said set is expressed, the patient survival rate is about 86%,
if at least one of the genes, or of the proteins, or of the antibodies of the set B is expressed, the patient survival rate is about 63%,
when said tumor is BAS:
if none of the 23 genes, or of the 23 proteins, or of the 23 antibodies of said set is expressed, the patient survival rate is about 65%
if at least one of the genes, or of the proteins, or of the antibodies of the set A is expressed, the patient survival rate is about 33%,
when said tumor is SQC:
if none of the 23 genes, or of the 23 proteins, or of the 23 antibodies of said set is expressed, the patient survival rate is about 88%
if at least one of the genes, or of the proteins, or of the antibodies of the set C is expressed, the patient survival rate is about 64%
when said tumor is LCNE:
if none of the 23 genes, or of the 23 proteins, or of the 23 antibodies of said set is expressed, the patient survival rate is about 65%
if at least one of the genes, or of the proteins, or of the antibodies of the set D is expressed, the patient survival rate is about 7%,
or,
during a period of time of 60 months after the diagnosis of said lung cancer
when said tumor is ADK:
if none of the 23 genes, or of the 23 proteins, or of the 23 antibodies of said set is expressed, the patient survival rate is about 82%,
if at least one of the genes, or of the proteins, or of the antibodies of the set B is expressed, the patient survival rate is about 46%,
when said tumor is BAS:
if none of the 23 genes, or of the 23 proteins, or of the 23 antibodies of said set is expressed, the patient survival rate is about 35%,
if at least one of the genes, or of the proteins, or of the antibodies of the set A is expressed, the patient survival rate is about 7%,
when said tumor is SQC:
if none of the 23 genes, or of the 23 proteins, or of the 23 antibodies of said set is expressed, the patient survival rate is about 77%,
if at least one of the genes, or of the proteins, or of the antibodies of the set C is expressed, the patient survival rate is about 42%
when said tumor is LCNE:
if none of the 23 genes, or of the 23 proteins, or of the 23 antibodies of said set is expressed, the patient survival rate is about 55%,
if at least one of the genes, or of the proteins, or of the antibodies of the set D is expressed, the patient survival rate is about 4%,
or,
during a period of time of 120 months after the diagnosis of said lung cancer
when said tumor is ADK:
if none of the 23 genes, or of the 23 proteins, or of the 23 antibodies of said set is expressed, the patient survival rate is about 76%,
if at least one of the genes, or of the proteins, or of the antibodies of the set B is expressed, the patient survival rate is about 39%,
when said tumor is BAS:
if none of the 23 genes, or of the 23 proteins, or of the 23 antibodies of said set is expressed, the patient survival rate is about 19%,
if at least one of the genes, or of the proteins, or of the antibodies of the set A is expressed, the patient survival rate is about 0%,
when said tumor is SQC:
if none of the 23 genes, or of the 23 proteins, or of the 23 antibodies of said set is expressed, the patient survival rate is about 58%,
if at least one of the genes, or of the proteins, or of the antibodies of the set C is expressed, the patient survival rate is about 33%,
when said tumor is LCNE:
if none of the 23 genes, or of the 23 proteins, or of the 23 antibodies of said set is expressed, the patient survival rate is about 40%,
if at least one of the genes, or of the proteins, or of the antibodies of the set D is expressed, the patient survival rate is about 0%,
wherein said gene, protein or antibody is determined as being expressed when:
either it is expressed in a sample of a patient afflicted by a lung cancer but not in a control sample of an healthy individual,
or it is expressed above a threshold corresponding to a background signal observed in a series of reference healthy tissues for each detection method, and
said gene, protein or antibody is determined as being not expressed when:
it is neither expressed in a sample of a patient afflicted by a lung cancer nor in a control sample of an healthy individual,
or it is expressed in a sample of a patient afflicted by a lung cancer at a level substantially equal or inferior to the level in a control sample of an healthy individual.

2. The element according to claim 1 of:
at least 4 genes chosen among a group of 23 genes comprising or consisting of the nucleic acid sequences SEQ ID NO 1 to 23,
or fragments of said genes
or complementary sequences of said genes
or sequences having at least 80% homology with said genes or fragment thereof,
or protein coded by said genes, said proteins comprising or consisting of the nucleic acid sequences SEQ ID NO 24 to 46,
said at least 4 genes being such that
a. at least one gene belongs to a first set A of 4 genes comprising or consisting of the nucleic acid sequences SEQ ID NO: 1 to 4,
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
wherein said element is suitable for the implementation of a prognosis method, preferably in vitro, of the survival rate of a patient afflicted by a lung cancer, said lung cancer being previously classified as a lung tumour belonging from the group consisting of ADK, SQC, BAS et LCNE,
wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
said prognosis being such that:
during a period of time of 30 months after the diagnosis of said lung cancer
when said tumor is ADK:
if none of the 23 genes of said set is expressed, the patient survival rate is about 86%,
if at least one of the genes of the set B is expressed, the patient survival rate is about 63%,
when said tumor is BAS:
if none of the 23 genes of said set is expressed, the patient survival rate is about 65%,
if at least one of the genes of the set A is expressed, the patient survival rate is about 33%,
when said tumor is SQC:
if none of the 23 genes of said set is expressed, the patient survival rate is about 88%,
if at least one of the genes of the set C is expressed, the patient survival rate is about 64%
when said tumor is LCNE:
if none of the 23 genes of said set is expressed, the patient survival rate is about 65%,
if at least one of the genes of the set D is expressed, the patient survival rate is about 7%,
or,
during a period of time of 60 months after the diagnosis of said lung cancer
when said tumor is ADK:
if none of the 23 genes of said set is expressed, the patient survival rate is about 82%,
if at least one of the genes of the set B is expressed, the patient survival rate is about 46%,
when said tumor is BAS:
if none of the 23 genes of said set is expressed, the patient survival rate is about 35%,
if at least one of the genes of the set A is expressed, the patient survival rate is about 7%,
when said tumor is SQC:
if none of the 23 genes of said set is expressed, the patient survival rate is about 77%,
if at least one of the genes of the set C is expressed, the patient survival rate is about 42%
when said tumor is LCNE:
if none of the 23 genes of said set is expressed, the patient survival rate is about 55%,
if at least one of the genes of the set D is expressed, the patient survival rate is about 4%,
or,
during a period of time of 120 months after the diagnosis of said lung cancer
when said tumor is ADK:
if none of the 23 genes of said set is expressed, the patient survival rate is about 76%,
if at least one of the genes of the set B is expressed, the patient survival rate is about 39%,
when said tumor is BAS:
if none of the 23 genes of said set is expressed, the patient survival rate is about 19%,
if at least one of the genes of the set A is expressed, the patient survival rate is about 0%,
when said tumor is SQC:
if none of the 23 genes of said set is expressed, the patient survival rate is about 58%,
if at least one of the genes of the set C is expressed, the patient survival rate is about 33%,
when said tumor is LCNE:
if none of the 23 genes of said set is expressed, the patient survival rate is about 40%,
if at least one of the genes of the set D is expressed, the patient survival rate is about 0%.

3. The element according to claim 2, wherein
a. said at least one gene belongs to a first set A of 4 genes comprising or consisting of the nucleic acid sequences SEQ ID NO: 1 to 4, is chosen among the nucleic acid sequence SEQ ID NO: 1 to 3,
b. at least one gene belongs to a second set B of 3 genes comprising or consisting of the nucleic acid sequences SEQ ID NO: 5 to 7, is chosen among the nucleic acid sequence SEQ ID NO: 5 or 6,
c. at least one gene belongs to a third set C of 6 genes comprising or consisting of the nucleic acid sequences SEQ ID NO: 8 to 13, is chosen among the nucleic acid sequence SEQ ID NO: 8 to 11, and
d. at least one gene belongs to a fourth set D of 9 genes comprising or consisting of the nucleic acid sequences SEQ ID NO: 14-23, is chosen among the nucleic acid sequence SEQ ID NO: 16 to 20.

4. The element according to claim 2, wherein
during a period of time of 30 months after the diagnosis of said lung cancer
when said tumor is SQC:
if none of the 23 genes of said set is expressed, the patient survival rate is about 88%
if one of the genes of the set C is expressed, the patient survival rate is about 73%,
if at least two of the genes of the set C are expressed, the patient survival rate is about 50%.
during a period of time of 60 months after the diagnosis of said lung cancer
when said tumor is SQC:
if none of the 23 genes of said set is expressed, the patient survival rate is about 77%
if one of the genes of the set C is expressed, the patient survival rate is about 59%,
if at least two of the genes of the set C are expressed, the patient survival rate is about 14%,
during a period of time of 120 months after the diagnosis of said lung cancer
when said tumor is SQC:
if none of the 23 genes of said set is expressed, the patient survival rate is about 58%
if one of the genes of the set C is expressed, the patient survival rate is about 45%,
if at least two of the genes of the set C are expressed, the patient survival rate is about 14%.

5. Prognosis method, preferably in vitro, of the survival rate of a patient afflicted by a lung tumour, from a biological sample containing said lung tumor belonging from the group consisting of ADK, SQC, BAS et LCNE, at a time from 30 to 120 months after the diagnosis of said lung cancer,
said method comprising a step of measuring, in said biological sample, the expression of
at least 4 genes chosen among a group of 23 genes comprising or consisting of the nucleic acid sequences SEQ ID NO 1 to 23,
or fragments of said genes
or complementary sequences of said genes said at least 4 genes being such that
a. at least one gene belongs to a first set A of 4 genes comprising or consisting of the nucleic acid sequences SEQ ID NO: 1 to 4
b. at least one gene belongs to a second set B of 3 genes comprising or consisting of the nucleic acid sequences SEQ ID NO: 5 to 7
c. at least one gene belongs to a third set C of 6 genes comprising or consisting of the nucleic acid sequences SEQ ID NO: 8 to 13, and
d. at least one gene belongs to a fourth set D of 9 genes comprising or consisting of the nucleic acid sequences SEQ ID NO: 14-23,
wherein
the nucleic acid sequences SEQ ID NO: 1 to 4 are expressed in BAS,
the nucleic acid sequences SEQ ID NO: 5 to 7 are expressed in ADK,
the nucleic acid sequences SEQ ID NO: 8 to 13 are expressed in SQC,
the nucleic acid sequences SEQ ID NO: 14 to 23 are expressed in LCNE,
said prognosis being such that:
during a period of time of 30 months after the diagnosis of said lung cancer
when said tumor is ADK:
if none of the 23 genes of said set is expressed, the patient survival rate is about 86%,
if at least one of the genes of the set B is expressed, the patient survival rate is about 63%,
when said tumor is BAS:
if none of the 23 genes of said set is expressed, the patient survival rate is about 65%,
if at least one of the genes of the set A is expressed, the patient survival rate is about 33%,
when said tumor is SQC:
if none of the 23 genes of said set is expressed, the patient survival rate is about 88%,
if at least one of the genes of the set C is expressed, the patient survival rate is about 64%
when said tumor is LCNE:
if none of the 23 genes of said set is expressed, the patient survival rate is about 65%,
if at least one of the genes of the set D is expressed, the patient survival rate is about 7%,
or,
during a period of time of 60 months after the diagnosis of said lung cancer
when said tumor is ADK:
if none of the 23 genes of said set is expressed, the patient survival rate is about 82%,
if at least one of the genes of the set B is expressed, the patient survival rate is about 46%,
when said tumor is BAS:
if none of the 23 genes of said set is expressed, the patient survival rate is about 35%,
if at least one of the genes of the set A is expressed, the patient survival rate is about 7%,
when said tumor is SQC:
if none of the 23 genes of said set is expressed, the patient survival rate is about 77%,
if at least one of the genes of the set C is expressed, the patient survival rate is about 42%
when said tumor is LCNE:
if none of the 23 genes of said set is expressed, the patient survival rate is about 55%,
if at least one of the genes of the set D is expressed, the patient survival rate is about 4%,
or,
during a period of time of 120 months after the diagnosis of said lung cancer
when said tumor is ADK:
if none of the 23 genes of said set is expressed, the patient survival rate is about 76%,
if at least one of the genes of the set B is expressed, the patient survival rate is about 39%,
when said tumor is BAS:
if none of the 23 genes of said set is expressed, the patient survival rate is about 19%,
if at least one of the genes of the set A is expressed, the patient survival rate is about 0%,
when said tumor is SQC:
if none of the 23 genes of said set is expressed, the patient survival rate is about 58%,
if at least one of the genes of the set C is expressed, the patient survival rate is about 33%,
when said tumor is LCNE:
if none of the 23 genes of said set is expressed, the patient survival rate is about 40%,
if at least one of the genes of the set D is expressed, the patient survival rate is about 0%.

6. Prognosis method, according to claim 5, wherein
a. said at least one gene belongs to a first set A of 4 genes comprising or consisting of the nucleic acid sequences SEQ ID NO: 1 to 4, is chosen among the nucleic acid sequence SEQ ID NO: 1 to 3,
b. at least one gene belongs to a second set B of 3 genes comprising or consisting of the nucleic acid sequences SEQ ID NO: 5 to 7, is chosen among the nucleic acid sequence SEQ ID NO: 5 or 6,
c. at least one gene belongs to a third set C of 6 genes comprising or consisting of the nucleic acid sequences SEQ ID NO: 8 to 13, is chosen among the nucleic acid sequence SEQ ID NO: 8 to 11, and
d. at least one gene belongs to a fourth set D of 9 genes comprising or consisting of the nucleic acid sequences SEQ ID NO: 14-23, is chosen among the nucleic acid sequence SEQ ID NO: 16 to 20.

7. Prognosis method, according to claim 5, wherein
during a period of time of 30 months after the diagnosis of said lung cancer
when said tumor is SQC:
if none of the 23 genes of said set is expressed, the patient survival rate is about 88%
if one of the genes of the set C is expressed, the patient survival rate is about 73%,
if at least two of the genes of the set C are expressed, the patient survival rate is about 50%.
during a period of time of 60 months after the diagnosis of said lung cancer
when said tumor is SQC:
if none of the 23 genes of said set is expressed, the patient survival rate is about 77%
if one of the genes of the set C is expressed, the patient survival rate is about 59%,
if at least two of the genes of the set C are expressed, the patient survival rate is about 14%,
during a period of time of 120 months after the diagnosis of said lung cancer
when said tumor is SQC:
if none of the 23 genes of said set is expressed, the patient survival rate is about 58%
if one of the genes of the set C is expressed, the patient survival rate is about 45%,
if at least two of the genes of the set C are expressed, the patient survival rate is about 14%.

8. Prognosis method, according to claim 5, wherein the step of measuring is carried out by using a technique chosen among the set consisting of:
a. Quantitative PCR,
b. DNA CHIP, and
c. Northern blot.

9. Prognosis method, according to claim 5, wherein the step of measuring is carried out by using nucleic acid molecules consisting of from 15 to 100 nucleotides molecules being complementary to said at least 4 genes.

10. Prognosis method, preferably in vitro, of the survival rate of a patient afflicted by a lung tumour, from a biological sample containing said lung tumor, at a time from 30 to 120 months after the diagnosis of said lung cancer,
said method comprising a step of measuring, in said biological sample, the expression of
at least 4 proteins chosen among a set of 23 proteins coded by 23 genes comprising or consisting of the nucleic acid sequences SEQ ID NO 1 to 23,
or fragments of said proteins,
said at least 4 proteins being such that
at least 4 proteins being such that
a. at least one protein belongs to a first set AP of 4 proteins comprising or consisting of the amino acid sequences SEQ ID NO: 24-27,
b. at least one protein belongs to a second set BP of 3 proteins comprising or consisting of the amino acid sequences SEQ ID NO: 28-30,
c. at least one protein belongs to a third set CP of 6 proteins comprising or consisting of the amino acid sequences SEQ ID NO: 31-36,
d. at least one protein belongs to a fourth set DP of 9 proteins comprising or consisting of the amino acid sequences SEQ ID NO: 37-46,
wherein
the amino acid sequences SEQ ID NO: 24 to 27 are expressed in BAS,
the amino acid sequences SEQ ID NO: 28 to 30 are expressed in ADK,
the amino acid sequences SEQ ID NO: 31 to 36 are expressed in SQC,
the amino acid sequences SEQ ID NO: 37 to 46 are expressed in LCNE,
said prognosis method being such that
during a period of time of 30 months after the diagnosis of said lung cancer
when said tumor is ADK:
if none of the 23 proteins of said set is expressed, the patient survival rate is about 86%,
if at least one of the proteins of the set BP is expressed, the patient survival rate is about 63%,
when said tumor is BAS:
if none of the 23 proteins of said set is expressed, the patient survival rate is about 65%
if at least one of the proteins of the set AP is expressed, the patient survival rate is about 33%,
when said tumor is SQC:
if none of the 23 proteins of said set is expressed, the patient survival rate is about 88%
if at least one of the proteins of the set CP is expressed, the patient survival rate is about 64%
when said tumor is LCNE:
if none of the 23 proteins of said set is expressed, the patient survival rate is about 65%
if at least one of the proteins of the set DP is expressed, the patient survival rate is about 7%,
or,
during a period of time of 60 months after the diagnosis of said lung cancer
when said tumor is ADK:
if none of the 23 proteins of said set is expressed, the patient survival rate is about 82%,
if at least one of the proteins of the set BP is expressed, the patient survival rate is about 46%,
when said tumor is BAS:
if none of the 23 proteins of said set is expressed, the patient survival rate is about 35%,
if at least one of the proteins of the set AP is expressed, the patient survival rate is about 7%,
when said tumor is SQC:
if none of the 23 proteins of said set is expressed, the patient survival rate is about 77%,
if at least one of the proteins of the set CP is expressed, the patient survival rate is about 42%
when said tumor is LCNE:
if none of the 23 proteins of said set is expressed, the patient survival rate is about 55%,
if at least one of the proteins of the set DP is expressed, the patient survival rate is about 4%,
or,
during a period of time of 120 months after the diagnosis of said lung cancer
when said tumor is ADK:
if none of the 23 proteins of said set is expressed, the patient survival rate is about 76%,
if at least one of the proteins of the set BP is expressed, the patient survival rate is about 39%,
when said tumor is BAS:
if none of the 23 proteins of said set is expressed, the patient survival rate is about 19%,
if at least one of the proteins of the set AP is expressed, the patient survival rate is about 0%,
when said tumor is SQC:
if none of the 23 proteins of said set is expressed, the patient survival rate is about 58%,
if at least one of the proteins of the set CP is expressed, the patient survival rate is about 33%,
when said tumor is LCNE:
if none of the 23 proteins of said set is expressed, the patient survival rate is about 40%,
if at least one of the proteins of the set DP is expressed, the patient survival rate is about 0%.

11. Prognosis method, according to claim 10, wherein
e. said at least one gene belongs to a first set AP of 4 genes comprising or consisting of the nucleic acid sequences SEQ ID NO: 24 to 27, is chosen among the nucleic acid sequence SEQ ID NO: 24 to 27,
f. at least one gene belongs to a second set BP of 3 genes comprising or consisting of the nucleic acid sequences SEQ ID NO: 28 to 30, is chosen among the nucleic acid sequence SEQ ID NO: 28 or 29,
g. at least one gene belongs to a third set CP of 6 genes comprising or consisting of the nucleic acid sequences SEQ ID NO: 31 to 36, is chosen among the nucleic acid sequence SEQ ID NO: 31 to 34, and
h. at least one gene belongs to a fourth set DP of 9 genes comprising or consisting of the nucleic acid sequences SEQ ID NO: 37 to 46, is chosen among the nucleic acid sequence SEQ ID NO: 37 to 43.

12. Prognosis method, according to claim 10, wherein
during a period of time of 30 months after the diagnosis of said lung cancer
when said tumor is SQC:
if none of the 23 proteins of said set is expressed, the patient survival rate is about 88%
if one of the proteins of the set CP is expressed, the patient survival rate is about 73%,
if at least two of the proteins of the set CP are expressed, the patient survival rate is about 50%.
during a period of time of 60 months after the diagnosis of said lung cancer
when said tumor is SQC:
if none of the 23 proteins of said set is expressed, the patient survival rate is about 77%
if one of the proteins of the set CP is expressed, the patient survival rate is about 59%,
if at least two of the proteins of the set CP are expressed, the patient survival rate is about 14%,
during a period of time of 120 months after the diagnosis of said lung cancer
when said tumor is SQC:
if none of the 23 proteins of said set is expressed, the patient survival rate is about 58%
if one of the proteins of the set CP is expressed, the patient survival rate is about 45%,
if at least two of the proteins of the set CP are expressed, the patient survival rate is about 14%.

13. Prognosis method, according to claim 10, wherein the step of measuring is carried out by using a technique chosen among the set consisting of:
a. western Blot,
b. ELISA,
c. Immunofluorescence, and
d. Immunohistochemistry.

14. Prognosis method, according to claim 10, wherein the step of measuring is carried out by using antibodies directed against said at least 2 proteins coded by said at least two genes.

15. Prognosis method, according to claim 10, further comprising a step of comparison of said measured expression to the expression in at least one control sample.


SEQUENCE LISTING (69 sequences)

SEQ ID NO: 1 - DNA, 240 bp, Homo Sapiens
atgtgttgcaactactacggcaactcctgtggctatggctccggctgtggctgtggctatggctctggctctggctgtggctgtggctatggaactggctatggctgtgggtatggctgtgggtttggctcccattatggctgtggttatggaactggctatggctgtggatatggctctggctctggctactgtggctaccggccattttgctttagaagatgctattcttcctgctaa

SEQ ID NO: 2 - DNA, 1405 bp, Homo Sapiens
atcttaagaggcgttcctttttgcatagttcccatgagcatgagagaagaagcaatgcacgctccggcagattcctaggaaccaaatacctctgaggagcaccagatttcagcttatgggatgctttgattgctctgtggctgcatttaggagaaggaagctgcagtcatgcgtcatcactgccagcctcacatctcttgacagttaaagccttagggtggagcaagggaaaatttaaaataacaaatgaagcaaaagcaagaggtgatgttccaaagcagaggaaggctaagtttatatatacaaatgtcaagtgtgtatagtgcaaaactaggaccagttggtggaatctgtggtcaaaaacaaaagccttcctttttttttttcaaggcccagtcccaagacgcaagaccacttgcgccagcagcgtgcatcagcaagatagcaaaagcaggacgagagctgcccggaagacatctacctggccagaagacacctaccctggccggaagacatgtacccctgaagatagagaaagaggccatcgtgtactacgtagcagtcatgtcagactgggacacttcctgtttacagaggactataaaacccctgtcctgtcctcacttggggctgacgccatcttaggcctcagcccgcctgcagccaggcgttcgttaaaacagcatgttgctccacaccgccttgtattgtttgttggtcccactctctgggctcgaaccaatacaagcacctttcaagcagtatattcttcagtgtcttgatcctccaaataactctcttctaattcctcctgaccacaaaaagcacttatactctaggatgactgattccagcccagtggcctggcaagggtgaattacaccttgcatatcacactcttgacatttgtgtgcgctagcataagaattataattgaaacagggatttaagtatctcctctctaggtgcctaccctccttggactcaggtcaaatttattaaaggaagttttgtttctagataggttgtttgaaataaaataacagaatgttcaagtaacacagtgtacctacagcttttaacaaaattgaggacttgggtctcgaaacaatttcctttgattttcaggtattttatctataaaaagggagataaagcattagttcataggacagttatatgtttaaatgtgataatgtatattaaccaccttgcatgtattcaaatgtgttttgaaatctaacgtctacattttgatagtttaactcttctacataagtgacttacaacaggcattaaatattgtttggcattttcatatatctgtaactgtatcttaatctacaatgagcttaattttaagtgtagcataaaacagaaccttcaataaagtggtaatattagg

SEQ ID NO: 3 - DNA, 857 bp, Homo Sapiens
gcttctcccagctggagtaggtgggggaggccagacatggaggcccttcctccagtcagatccagccttttggggatcctgttgcaggttacgaggctctcagtgctgttggttcagaaccgagatcacctctataatttcctgctcctcaagatcaacctcttcaaccactgggtgtcagggctggcccaggaggcccgggggtcctgtaactggcaggcccacctacccctgggagctgcagcctgccccctgggccaggctctctgggctgggctggctctgatacaggtccccgtatggctggtgctacagggacccaggctgatgtgggctggcatgtggggcagcaccaagggcctgggcctggccttgctcagtgcctgggagcagctgggcctgtctgtggccatctggacagatctgtttttgtcatgtctgcacggcctgatgttggtggccttgctcttggtggtagtgacctggagggtgtgtcagaagtcccactgcttccgactgggcaggcagctcagtaaggccttgcaagtgaactgcgtggtaaggaagctcctggtacagctgagacgtctgtattggtgggtggagactatgactgccctcacctcctggcacctggcctatctcatcacctggaccacctgcctggcctcccacctgctgcaggctgcctttgagcacacgacccagctggccgaggcccaggaggttgaaccccaggaggtctcagggtcttccttgctgccctcactgtctgcgtcctcggactcagagtctggaacagttttgccagagcaagaaactcccagagaataaatgtatccccatctgcctctaaa

SEQ ID NO: 4 - DNA, 2521 bp, Homo Sapiens
aaacgagtggagacacgaggaccagcgcgagcggtcccggtgggctaccctccccctgcgacgaccccccctcgctctgaccgactggtcccctaaacggtggcggcggtttttggtcgttgggccccgggatttaggaccaacatttgaagacccgaaggggaactgcaaccatgaatgaagaaaatatagatggaacaaatggatgcagtaaagttcgaactggtattcagaatgaagcagcattacttgctttgatggaaaagactggttacaacatggttcaggaaaatggacaaaggaaatttggcggtcctcctccaggttgggaaggtccacctccacctagaggctgtgaagtttttgtaggaaaaatacctcgtgatatgtatgaagatgagttagttcctgtatttgaaagagctgggaagatatatgaatttcgacttatgatggaatttagtggtgaaaatcgaggttatgcttttgtgatgtacactacaaaagaagaagcccaattagccatcagaattcttaataattatgaaattcgaccagggaagtttattggtgtgtgtgtaagcctggataattgtagattatttattggagctattcccaaggaaaagaagaaagaagaaattttagatgaaatgaagaaagttacagaaggagttgtagatgtcattgtttatccaagtgcaactgataagaccaaaaatcgtggttttgcatttgtggaatatgaatctcacagagctgctgctatggcaaggaggaaactaattccaggaacattccaactatggggccacaccattcaggtagattgggctgacccagagaaagaggtggatgaggaaaccatgcagagagttaaagttctttatgtaagaaatttaatgatctcaactacagaggaaacaattaaagcagaattcaataaatttaagcctggtgcagttgaacgggtaaagaaacttagagattatgcttttgttcactttttcaaccgagaagatgcagtggctgccatgtctgttatgaatggaaaatgcattgatggagcaagtattgaggtaacactagctaaaccagtaaataaagaaaacacttggagacagcatcttaatggtcagattagtccaaattctgaaaatctgattgtgtttgctaacaaagaagagagccacccaaaaactctaggcaagctgccaactcttcctgctcgtctcaatggtcagcatagcccaagtccgcctgaagttgaaagatgcacttaccctttttatcctggaacaaagcttactccaattagtatgtattctttaaaatccaatcattttaattctgcagtaatgcatttggattattactgcaacaaaaataactgggcaccaccagaatattatttatattcaacaacaagtcaagatgggaaagtactcttggtgtataagatagttattcctgctattgcaaatggatcccagagttacttcatgccagacaaactctgtactacgttagaagatgcaaaggaactggcagcccagtttacattacttcatttggactacaatttccatcgcagctcaataaatagtctttcccctgttagtgctaccctctcttctgggactcccagcgtgcttccttatacttcaaggccttattcttatccaggctatcctttgtcaccaacaatatcacttgctaatggcagccatgttggacagcggctatgtatctccaatcaggcctccttcttctgaagaaaatactaacattagtatgaaaatttgtgtaaatttgtagtatgaaaacttgcaaattaaaatattgttttattttagaatcgggtttgcatatttggttttaaaaaggtatttattccaaagtactaaacatcagctataattcagaataacatggagttgtagaatttataaaaatgcaaagtttaaaaagttattcagtggtttctcttgataaaggtacagcaaactactattctttttaaacttctaggattttcttctactttctgagtgggcaatagaacctagtcatttatgtttttttttttttttgcataattttactaaatagtatttcacaaatattaaagcacttgaagacaatggttatagtagatttgattaccaaggatcactatctgtactggagattagaacaattatatgaccagaagcatctaaccattatgtaaaaagaaatgatgagacaaaaagattaagatacaaattttgtgcagtactaaagaaaaagcagtctaccattgtggtccttgaaaataactatagatatttttgttatttgttagacacaaattataattttgttgttaatgtatttaagcattttatagttatgctttgtgtttttgatattctttgtattgttaataacaagtgttatgggtttttaatgttgaaatcatgtgttaatttttgtacttgaattcaaattttttgacattaaatatgtgatgcttcta

SEQ ID NO: 5 - DNA, 3305 bp, Homo Sapiens
cacaggcaggccgtacctcgaaatcccagcggggacacctgtcagggagacgcaccctcacaccgcccatgcatgcccgacgcatctttgggatccttagggatcacgtggtgcttccttgaatctccgcttgaggtttcttcaggcaggttcggccttgcccgactcctaggatcgcaggaccatggcgacgatcccgcggagaggggacgcactgccacagacgcctgggggccaagccgctggggccagtcccccggcaacggtggcggttactgtgacgcgagtcccccctcagcgctagcgcctggtgatcgcgcctgggctcttcctgcgtctccaagctcaggggctcctgcttctcagcactgctgtctggaaaaggcgggaacgaggactaaggcaagcccagtgtggggcagagatggcaacacctggaactagcttttcatgggggcagataatgtggagagtgtcccaattacatctccctttattctatcccatactactaaaccccaccagctatttgcctacaaactgtgtatcagtcaggacatcaggacaccaagataaaggactagagtttggtaatcaactactggctcctccagcagctcagaatgtggcctgtatgcatcacttcacttaattcattcaagaaatgctgactgagttcctgctgtgtgttggacactttcttagaagtgggacacactgcagtgtgtgggacaaaggctgggccgctcattgcttatgttctagttgaagagagagtcaatcaacaacattctactttcagacattgataaatgctgtcatgaaaagacaaggaacatgaaaaggtaagaaaagggatggagagagatgggatgaggggtgcctgggcagacaatgcaatgatctggcaaaagagtaggacaatcatgagactgcatgtgttccagtttctgctgctgtaaaacagcatccacaggttacatctttactattctgttacatacatactattatttaaataaaaaatagtactagttaactgtgaaaattgcaattaatagggcagattaaacaaatcctttaaatttaaacaaatgttttatggttttttatcctctgatacattgtttcaactttaaaaaaccctctttatttgatatttaaactctatattaatgtatattcttaagatacataaatttaagatttttttcgagacagggtctcattctgtcacacagtctggagtgctgtggtgcaattatggttcactgcagactccacctctccaggctcagataatcctcctgcctcagactcctgagtatctgggaccacaggtgtgcatcaccacacccagctaatttcttgtatttttttgttgggatggggttttgcgatgttgcccaggctggtctcaaactcctgggctcaagtgatctgcctgccttggtgtctcaaagtgttgggatcacaggcgtgagccaccacatccagcctaatttaagatttttaaaaagcagaatgtaaatatggtcatgcattgtgtaattctgtggatactttctgagtaatgcatggttaggtgacttcatcattatgtgaacattatagagtgtacatacacaaacctagatgatgtagcctgatggtacagcctatcacttcgaggctacaaatccaaattgcctggatactgtaaacaattatactcaatggtatttgtgtatctaaacatgtctaaacatagaaagggtacagtaaaaatatggtataaaagataagaaatcgtataccggtatacagcacttaccgtggatggagcttgcaagactggaagttactccgggtgagtcagtgactgagcggtgagtgaatgtgaaggctagggcagtactgtatactactgtagactttatatgtactgtatgcttaggctacactaaatttatttgaatttttttgtcggtaataaattaaccttagcttactataatttctttataaaccttttaatttttaaaaatgttttgactctcttgtaataaattagcttcaaacacattgtatagctgtacacaaatattttctttatatccttattctataatcttctacttaaaaatttttttaatttttttattttaatttaattttattttattttgagacagagtctcactctctcacccaggctggagtgcagtggcgtgatcttaactcactgcaacctccgcttcccaggtaatattatttaaataaaaatagtactagttaactgtgaaaattgcaattaatagggcagatcaagcaattctcttgcctcagcctccagacttttttgttaaaaactaagacacaaacacattagcctaggtacacacagtgccaggatcatcaaaatgtcattaggcagtataaacttttcagctccataaaagttggaaaaaaaataataataataaatacaattttataaaaagaaaattttcagctctgctataatcttatgggaccactactgtatatgtggtccctcattgaccaaaacatcattatgtggcacatgattataacaaatgatggatggcgaggttgtgaagaaactggaacccttgtgcattactggttgtaatataaaaaggtacagccactgtaaaaaaaaaaacacagtttggttatttcttcataagttaatcatagaattaccatataatccagcaatttcacttctaggtatatgcccaaaataattgaataaggtgtttaaacataaacttgtacaagaatgttcatagcagtacatttcacaatactcagataatcctctcattggaagcaactcaaatgtccaccaactgatggtattacccatgcaatggaatattattcagtcataaaaaggaatgaagtattgaggcatgttacaacatgaaggaactttgaggacagtttgttaatacgctatatactgtgtaattctatttatatgaaatatccagaataggtaaatcattagagactagagacagcagattagtggatgccaggagtttggggaaggaaggggtgggaagaatgggaagtgactgcttaatggatacagggtttctttttggggtgatgaaaaatttctggaactagattgtagtgatggttgcacaacaccgtaagtgtaataatgtcactgaattatacactttaaagtgatttaaatggtaaattgtattatttatgtattttaccacaataaagaagtcacctatcaaaaaaaaaaaaaaaaaagaaaaaaaaaaaaaaaaaaa

SEQ ID NO: 6 - DNA, 2499 bp, Homo Sapiens
cttctaattctgttattgcaactgcagaccgttacctggtacgctggctgctacctccctcactcttgtcagagtcggagctacaggcagtgccttcagctctgagctcaggcatcccggtccctgtttttgcggttaaggactctaaagtgttgtgtcgtgttcatcaactttttctcaacttccctggctctacctcttctgccacaaacgtcagcatggtggtatctgccgaccctttgtccagcgagagggcagagatgaacatcctagaaatcaaccaggaattgcgctcgcagctggcagagagcaatcagcagttccgagacctcaaagagaaattccttataactcaagctactgcctactccctggccaaccagctgaagaaatacaagtgtgaagagtacaaagacatcatagactctgtgctgagggatgaactgcagtccatggagaagctggcagagaagctcaggcaagctgaggagctcaggcagtataaagccctggttcactctcaggcaaaagagctgacccagttacgggagaagttacgggaagggagagatgcctcccgctggctgaacaagcatctgaaaaccctcctcactcctgatgaccctgacaagtcccagggtcaggacctccgagagcagctggctgaggggcacaggctggcagagcaccttgttcacaagctgagcccagaaaatgatgaagatgaagatgaggatgaagacgacaaagacgaggaggttgagaaagtacaggaatcacctgcccccagagaggtgcagaagactgaagaaaaggaagtccctcaggactcactggaggaatgtgctgtcacttgttcaaatagtcacaacccttctaactccaaccagcctcacaggagcaccaaaatcacatttaaggaacacgaagtcgactctgctctggttgtagagagtgaacaccctcatgatgaagaggaggaagctctaaacattcccccagaaaatcaaaatgaccatgaggaggaggaggggaaagcgccagtgccccccagacaccatgacaagtccaactcttaccggcatcgtgaagtctctttcttggcattggatgaacagaaagtttgctccgctcaggatgttgccagggattactccaatcccaaatgggatgaaacctcacttggcttcctcgaaaagcaaagtgatcttgaagaggtgaaaggacaagaaacagttgctcccaggctcagcaggggaccgctgagagtggacaagcatgaaatcccccaggagtcactggatggatgttgcttgactccttccatccttcctgacctgactccctcctaccacccttattggagcactttgtactcttttgaagacaagcaagtcagcttggctcttgtagacaaaattaaaaaggatcaagaggagatagaagaccaaagcccaccatgccccaggctcagccaggagctgccagaggtgaaggagcaggaagtcccagaggactctgtgaatgaagtttacttgactccctcagttcaccatgacgtgtctgactgccaccagccttatagcagcaccttgtcctcattggaggatcagcttgcctgctctgctctggatgtagcctcccccaccgaggcggcctgtccccaagggacttggagtggagacttgagccaccaccagtcagaggtgcaagtttcacaggcacagctggaaccaagcaccctggtgcccagttgtctgcgactacagctggatcaagggttccactgtgggaacggcttggcccagcggggcctttcctccaccacctgcagcttctcagccaatgctgattctgggaaccaatggcccttccaagagctggttttagagccctctctggggatgaagaaccctccccagctggaagatgatgcacttgaaggctcagcaagcaacacacaagggcgtcaagtcactggccggattcgtgcctcccttgtcctgatactgaagaccatcagaagaagactcccgttcagcaagtggagactggcattcagattcgctggcccgcatgctgagagcgcagagataccaaatactgctggaaggacgcaaaggatggcaggatgaaagaatgtcacaaaaagcagcttttccacttgataaaaacaactaaaacagcaaagcaagtttaagtccaaacacaatactgcaggggtccttcactgaggattgaatttcagacacagaatactcttgatgacttcaagccactatgctcctttgatttgagaagccacattccatccccctccaattgtgatcaatacctagggagaccaatgcccagatggacaaatagcattgaccggcgttagccctgtttctcaattcccatcgtgtagagaacaggagtccgcagctgctggcaggagacagcatgtcagccgggactctgccagggcagagtatgagcaatgccatgttcttgctg

SEQ ID NO: 7 - DNA, 6093 bp, Homo Sapiens
acgccgccaactggcgggggtgcgggggagacaataatttgttccgcggtaataagaacggtgactgctggccgtggatccatttcacaggcctgccttctctcactaacgctcttcctagtccccgggccaactcggacagtttgctcatttattgcaacggtcaaggctggcttgtgccagaacggcgcgcgcgcgcgcacgcacgcacacacacggggggaaacttttttaaaaatgaaaggctagaagagctcagcggcggcgcgggcgctgcgcgagggctccggagctgactcgccgaggcaggaaatccctccggtcgcgacgcccggccccggctcggcgcccgcgtgggatggtgcagcgctcgccgccgggcccgagagctgctgcactgaaggccggcgacgatggcagcgcgcccgctgcccgtgtcccccgcccgcgccctcctgctcgccctggccggtgctctgctcgcgccctgcgaggcccgaggggtgagcttatggaaccaaggaagagctgatgaagttgtcagtgcctctgttgggagtggggacctctggatcccagtgaagagcttcgactccaagaatcatccagaagtgctgaatattcgactacaacgggaaagcaaagaactgatcataaatctggaaagaaatgaaggtctcattgccagcagtttcacggaaacccactatctgcaagacggtactgatgtctccctcgctcgaaattacacggtaattctgggtcactgttactaccatggacatgtacggggatattctgattcagcagtcagtctcagcacgtgttctggtctcaggggacttattgtgtttgaaaatgaaagctatgtcttagaaccaatgaaaagtgcaaccaacagatacaaactcttcccagcgaagaagctgaaaagcgtccggggatcatgtggatcacatcacaacacaccaaacctcgctgcaaagaatgtgtttccaccaccctctcagacatgggcaagaaggcataaaagagagaccctcaaggcaactaagtatgtggagctggtgatcgtggcagacaaccgagagtttcagaggcaaggaaaagatctggaaaaagttaagcagcgattaatagagattgctaatcacgttgacaagttttacagaccactgaacattcggatcgtgttggtaggcgtggaagtgtggaatgacatggacaaatgctctgtaagtcaggacccattcaccagcctccatgaatttctggactggaggaagatgaagcttctacctcgcaaatcccatgacaatgcgcagcttgtcagtggggtttatttccaagggaccaccatcggcatggccccaatcatgagcatgtgcacggcagaccagtctgggggaattgtcatggaccattcagacaatccccttggtgcagccgtgaccctggcacatgagctgggccacaatttcgggatgaatcatgacacactggacaggggctgtagctgtcaaatggcggttgagaaaggaggctgcatcatgaacgcttccaccgggtacccatttcccatggtgttcagcagttgcagcaggaaggacttggagaccagcctggagaaaggaatgggggtgtgcctgtttaacctgccggaagtcagggagtctttcgggggccagaagtgtgggaacagatttgtggaagaaggagaggagtgtgactgtggggagccagaggaatgtatgaatcgctgctgcaatgccaccacctgtaccctgaagccggacgctgtgtgcgcacatgggctgtgctgtgaagactgccagctgaagcctgcaggaacagcgtgcagggactccagcaactcctgtgacctcccagagttctgcacaggggccagccctcactgcccagccaacgtgtacctgcacgatgggcactcatgtcaggatgtggacggctactgctacaatggcatctgccagactcacgagcagcagtgtgtcacgctctggggaccaggtgctaaacctgcccctgggatctgctttgagagagtcaattctgcaggtgatccttatggcaactgtggcaaagtctcgaagagttcctttgccaaatgcgagatgagagatgctaaatgtggaaaaatccagtgtcaaggaggtgccagccggccagtcattggtaccaatgccgtttccatagaaacaaacatccccctgcagcaaggaggccggattctgtgccgggggacccacgtgtacttgggcgatgacatgccggacccagggcttgtgcttgcaggcacaaagtgtgcagatggaaaaatctgcctgaatcgtcaatgtcaaaatattagtgtctttggggttcacgagtgtgcaatgcagtgccacggcagaggggtgtgcaacaacaggaagaactgccactgcgaggcccactgggcacctcccttctgtgacaagtttggctttggaggaagcacagacagcggccccatccggcaagcagataaccaaggtttaaccataggaattctggtgaccatcctgtgtcttcttgctgccggatttgtggtttatctcaaaaggaagaccttgatacgactgctgtttacaaataagaagaccaccattgaaaaactaaggtgtgtgcgcccttcccggccaccccgtggcttccaaccctgtcaggctcacctcggccaccttggaaaaggcctgatgaggaagccgccagattcctacccaccgaaggacaatcccaggagattgctgcagtgtcagaatgttgacatcagcagacccctcaacggcctgaatgtccctcagccccagtcaactcagcgagtgcttcctcccctccaccgggctccacgtgcacctagcgtccctgccagacccctgccagccaagcctgcacttaggcaggcccaggggacctgtaagccaaacccccctcagaagcctctgcctgcagatcctctggccagaacaactcggctcactcatgccttggccaggaccccaggacaatgggagactgggctccgcctggcacccctcagacctgctccacaatatccacaccaagtgcccagatccacccacaccgcctatattaagtgagaagccgacaccttttttcaacagtgaagacagaagtttgcactatctttcagctccagttggagttttttgtaccaacttttaggattttttttaatgtttaaaacatcattactataagaactttgagctactgccgtcagtgctgtgctgtgctatggtgctctgtctacttgctcaggtacttgtaaattattaatttatgcagaatgttgattacagtgcagtgcgctgtagtaggcatttttaccatcactgagttttccatggcaggaaggcttgttgtgcttttagtattttagtgaacttgaaatatcctgcttgatgggattctggacaggatgtgtttgctttctgatcaaggccttattggaaagcagtcccccaactacccccagctgtgcttatggtaccagatgcagctcaagagatcccaagtagaatctcagttgattttctggattccccatctcaggccagagccaaggggcttcaggtccaggctgtgtttggctttcagggaggccctgtgccccttgacaactggcaggcaggctcccagggacacctgggagaaatctggcttctggccaggaagctttggtgagaacctgggttgcagacaggaatcttaaggtgtagccacaccaggatagagactggaacactagacaagccagaacttgaccctgagctgaccagccgtgagcatgtttggaaggggtctgtagtgtcactcaaggcggtgcttgatagaaatgccaagcacttctttttctcgctgtcctttctagagcactgccaccagtaggttatttagcttgggaaaggtggtgtttctgtaagaaacctactgcccaggcactgcaaaccgccacctccctatactgcttggagctgagcaaatcaccacaaactgtaatacaatgatcctgtattcagacagatgaggctttccatgggaccacaactattttcagatgtgaaccattaaccagatctagtcaatcaagtctgtttactgcaaggttcaacttattaacaattaggcagactctttatgcttgcaaaaactacaaccaatggaatgtgatgttcatgggtatagttcatgtctgctatcattattcgtagatattggacaaagaaccttctctatggggcatcctctttttccaacttggctgcaggaatctttaaaagatgcttttaacagagtctgaacctatttcttaaacacttgcaacctacctgttgagcatcacagaatgtgataaggaaatcaacttgcttatcaacttcctaaatattatgagatgctggcttgggcagcatccccttgaactcttcactcttcaaatgcctgactagggagccatgtttcacaaggtctttaaagtgactaatggcatgagaaatacaaaaatactcagataaggtaaaatgccatgatgcctctgtcttctggactggttttcacattagaagacaattgacaacagttacataattcactctgagtgttttatgagaaagccttcttttgggggtcaacagttttcctatgctttgaaacagaaaaatatgtaccaagaatcttggtttgccttccagaaaacaaaactgcatttcactttcccggtgttccccactgtatctaggcaacatagtattcatgactatggataaactaaacacgtgacacaaacacacacaaaagggaacccagctctaatacattccaactcgtatagcatgcatctgtttattctatagttattaagttctttaaaatgtaaagccatgctggaaaataatactgctgagatacatacagaattactgtaactgattacacttggtaattgtactaaagccaaacatatatatactattaaaaaggtttacagaattttatggtgcattacgtgggcattgtctttttagatgcccaaatccttagatctggcatgttagcccttcctccaattataagaggatatgaactgagtttttcttttgttgtttgttcttagctgtaattcctatgcttctatttcagagagccaggagagtttgatattaaaggaggttaaaactgtgatcttatgccatgtcatcaatggccacttaggggccatggctgatgacacattcttatctctacagtactaatgtgttattatagagccatgcattttatttctgaataagaacatatttaaactaatattcccttacaatatggacagtattaatccttccaagatgcagtatttatcaagtgaagcatatttagcagcaaattccattttaacataacttaggaaccaataaccagggtgttttgtggttgggggaggcacggggtggagtattcttttttatatcctcaaaacaaaaaaaatcaatacttatatttcaatggcaatctagtatttttttaaaagactgtataggcatgaataatagaggtggtttgagttttgtagggccatcacctggaaagtcaatgtgactagacacaaagtagcccagaggctacttttcttcctacagcttattatagttgtaggttctatgacctcacttcatgggttccaggcaattccgctgaaaggtttgtctcctgaaattttttaagtttgttttcctgacacatgtaatcagatgtgtagcaaccgagggaaacgaagcctaacattctccattgtggaaatacacacaggaggttacatttcacagcgtggatttttccagcttacacatgtgggatgacatcacagaaaccacaaaagcagcaaattaaactgtaggagagtcaatactcctgacgagtctcggggggggggcatttttatgccttcttaactttatgagaattctcaggctgaactataggccattgttcccaggcaaatcaatacatcaatgcatcctcaaaaaaaaa

SEQ ID NO: 8 - DNA, 2714 bp, Homo Sapiens
ggggctggcattcagtgaggctgggggagaagcttcctaaaagcattgccattgaaaagcagggcatacctatctgatactgaggatgtagaatgcagtttggagatgcttcctagctacctaatcttccaaggcaaaccatgcactcttcatcctgatatttgtgatgcctccctgggctacatgacagagggtgcactgcaaaggatgtgaaggaaagtcaccacagggcaaaggtggaaacccaggggcctttccccagtgagactttctggctgttcctttggaacttccacctgctagttgcagatgtttgccactggatggggccccatgctacgtgggaactgttcaggactgtgggtacttagttccagcagagctggctccaccagggcaccaggactgcaggcttctcaactcctggcctggagaggcagcttcagcaaccagtgcctgggaatccactagccctttctctgtgagtcacgtgagggcagaatctgctcaaagtcagagggctggagcaacctggattgtgccagagtgagggcccagctcactggaggtcagagtaagagagtcaaggatgggaagaaacagggttttgctcagtcttgcagatactgaggcactcaggaggctcaggatggcagcaacagggcatgtgctaaacttaccctagcctcttccttttaggcagccaccacagaggacatctgtttgcctctgtaccctaccccatctccatgctggtagatatagtctggggatacctttaaaacaaacaaacaaacaaacaaaacaggatctgtctatcacctgtccaaatcctgaagcaaaatgagccttctttatggccaccttggaaaggcctgagtccccagcatcacctgggatacaaagtgttctgaggtgggggcagagtgccctcctgcccagatgccacactccacaatatctcttcctggcagacaggtttctcttaacttcctttggcaaatttggtttcacttccttttgcaactttagaacctttctcaggtcagtcataaggtagtggggaagccaggatttgaacccattctgtctgattccagagctcatgcattaaccacctgactgtggtgcctccccatcatatgtgcatcaagtatgatcatttttgtagttctcagagtacttagcaccatgcacatctgtaataagtacagccactgcctactgggaggtctaaaggttcaactcataagggacccacaggccacactgggctccagtctccttaaaagatgtgggacggagatcaacaattgtcagcagaaccccatcaggcatttgtcttctcactgagccttgcagcaaaccacacagctgtctataacctatctgttcccagattcaagaacacaagctgctcatcagttgggtgcaggatccactctagtacaaaagctgtgtgtctcaaggcataattccttatgattacttcatagctctgcttttgtcttgagacaaaagaaatttccttcaattcctacttttgctgagatattgttttttgaaaagcaattaaatcttgtcaatcacttttcccacctctttgagatacttacatggcttttcttctttattctcttagtgtggtaaattacattgatttattttaaatatgaaaccagccttgaattccaggaacaaactccatttggacatgctagaacaaccttttatatttgtttagatttgatttcctgaagtggtgttaaaaggttttcatatctatgtctgtgagggatattaatctgtacatttttttattactattttctggtgttatggtaatgctagtctcatagaatgaattaggaaaagttgcctccttctgtattttctgagagtttgtgtggcactggtaaaatattttctgtatatgtttgacataattcaccaggaaaattatttgtacctacaagttccctcctggaaagatgttgataatcacttattatcctttcattgtctgccagatctgtggcattgcttttttctctggtattagtaatttaaattttcccttatttcttgatcaattttggcagagatttatcaattttattaatctcttttaaaaatctgtattgagctttgttaattttctctattacatactttttatttctactcttacgattgctatttctttatttctccttcggatttagcccatttttctatttgtctaattttaggatttaaaattcaatcattatttttagatatttttatttttaaatataaacatgaaagttgcaatattaccttgggcactgttttgactgcattccacaagtttttatttgtcatgttttaatcaccattcagttcaaaatactttctaatgctccttgttatttcttctttgacccattggttatataaaagtacgttttgctttatttctaaatatgtgaagactttctagatatctcagttatttattactaatttaattttattgtagccagagaaaatacactgtatgtgttcaatctttggaaatttcttgagacttattttttgacccagtatgtttgccctcatggacattccatattcactagaaaaagaatgtgtgttcaaataatgttggatatagtgttatgcaaatgccactaaatcaagataaatcttttgcaaaaaaaaaaaaaaaa

SEQ ID NO: 9 - DNA, 3280 bp, Homo Sapiens
gggggagggatgactaaagacaacggctgtaagagaactccacaagagagtcagaacgaaaatgtgcaacaagtgcggcggctcctaccatggcaggtgattcgaggactcagcacgaggcgagagtagggaccaggaagagccggaaaacccgcctgtgattggccgtccacgggtatcggtcgttgtgattgggtgaggcccagacagacagctgcgttttgaaccgcgtagggttctgggtagcaaaggccttgcaaggctcttaaccgaaagggggagggggaaggtcgccaacaaacggctgagctcacaatcctggccggggcgtccccctcccccatggagagagccagacccgagccgccgcctcagccgcgcccgttgcgtcccgctccgcccccgctgccggtcgagggcacctccttttgggcagcagccatggagccccctccgtcgtctcccacactgagcgcggcagccagtgcgaccttggcctcgtcgtgcggggaggcagtggcgtccggcttacagcccgcggtgcggcggctgctgcaggtgaagccagagcaggtgttgctgctaccacagcctcaggcccagaacgaggaagccgctgcctcgtccgcgcaggcgcggctgttgcagttcaggcccgacctgcggctcctgcagccgccgacagcgtcagacggcgccacctccaggcccgagttgcacccggtgcagcccctggcgctgcatgtcaaggccaagaagcagaagctggggcccagcctggatcagtcagtggggcctcgaggggccgtcgaaaccggtcctagagcctccagggtggtcaagttggaaggccccgggccggccctcggctacttccgaggggacgagaagggcaagctggaggcggaggaggtcatgagagactcgatgcaaggcggggcaggcaaaagcccggcagccatccgagaaggtgtgatcaaaacggaggaacccgagagactcctcgaggactgcaggctcggcgcggagcccgcgtccaatggcctggttcatggcagcgcggaggtcatcttggccccaacgtccggtgcctttgggccgcaccagcaagaccttaggatccctttgacgctccacacggtcccccctggggcccggatccagtttcagggagctccgccttcagagctgataagattgaccaaggtccccctgacaccagtgcctactaaaatgcagtccctactggagccttctgtaaaaattgaaaccaaagatgtcccgctcaccgtgttgccctcagatgcaggcataccagatactcccttcagtaaggacagaaatggtcatgtgaagcgacccatgaacgcatttatggtttgggcaaggatccaccgaccagcactagccaaagctaacccagcagccaacaatgcagaaatcagtgtccagcttgggttagagtggaacaaacttagtgaagaacaaaagaaaccctattacgatgaagcacaaaagattaaggaaaagcacagagaggaatttcctggttgggtttatcagcctcgtccagggaagcgaaaacgattccctctaagtgtttccaatgtattttctggtaccacacagaatatcatctctacaaatcctacaacagtttatccttaccgctcacctacgtactctgtggtaattcccagcctacagaatcccatcactcatccagttggtgaaacctcacctgctatccagctgcccacacctgcagtccagagcccaagccctgtcacacttttccagcccagcgtctccagtgctgctcaggtggctgtccaggatccaagtctacctgtctatccagcactcccaccccaacgctttactgggccttcccaaacagacactcatcagctgcattctgaagccactcacactgtgaagcaacccactcctgtctctctagagagcgccaacaggatttcaagtagtgcaagtactgcccatgccagatttgcaacttcgaccatccaacctcctagggagtattccagcgtttccccttgtcccagaagtgctccaatcccccaggcttctcccattccacacccacatgtctaccagccccctccccttggccatccagccacactgttcgggacaccaccaagattctcttttcatcacccttacttcctacccggacctcactacttcccatcaagtacatgcccttacagtcggcctccctttggctatggaaattttccgagttcaatgccagaatgccttagttattatgaagacaggtacccaaaacatgagggtatcttttcaactttaaatagagactattcttttagagactactcaagtgaatgcacacacagtgaaaattctcggagttgtgagaacatgaatggaacttcttactataacagtcatagccacagtggggaagaaaacttaaaccctgtgcctcagctggacattggaaccttggagaatgtcttcacagccccgacatcaactccttctagcatccagcaagtcaatgtcaccgacagtgatgaggaggaagaagaaaaagtgctcagggatttataattttaaaacaaatatgcacagaaaataaacatttcttaaaatatattctgggtcagttggtatgagaaaaaaaaaagcctagaattctttgttgaaagttttcagtcgtgatttgaggagttaaaaccaaatgcaatttatgtcttcataaaattttgattagtgaaactagagtctggatgtttcattgtaggaatatttaagttattaagtagtttaattttaatggctgaaatttgcatcaacatgtattattattactttatcctggaacatgcaaaatactgaagcctcacagttgtatgtgaggggaaaggggaaataaatctagcatagtgtgatttttattttatctcaggatacattttttaaatgattttttgtttgctttttatgtaatacttatggatgttgtcaatttttgatgtaacattttgaaagtattttgacaactcctagtgaacttggacttggttgctaaatttaacttacactaataaccaattataagttccaaatgtgttttaatggcacctgggtgattcttcagctaaatttagtcatttctgtttctaaatatttttatcattttaaaatattttttttccatttggcatacatcgttctttgttgtaattaaataaacatagataaaattgttaaaaaaaaaaaaaaa

SEQ ID NO: 10 - DNA, 2919 bp, Homo Sapiens
agttgagattcagaccttgacagttgtaaccgatagactgggctggggcaacgctgatgaaaggcaaattcctgagagaaaaatcacccagactttctagaagttgttatgagtggacctgactacctagagagagtaatacagaagctccgtgccgatcacagctacctcattaccatgttcctccaagatggccgcccctgaggagaatcatcaactgggctttacggaagagagaaatccaaacgaccatcagcaacaaatgaaagactgaagttgaaaggaaatccataatttttgaatgctggccgaggtctgtgacctctgactgaatggtttccaagctccgaattcctaggtgatgttttcatttgttcacacttgctgaagaagggaaaggctctctgatttacgtgtaagcaaaacaatctaaacagtaggataaaatcatgttggaatggagttctgcgtggatgctcctttgaaccaaatcagttccacaactgaccctgtcagggacatataaatttagcattttggtgtggatacagaagacgagattaacacagatcaagtaacatggaaataaaaaaggagacaatgctggatgatacaggggcagccaacaagaggagaaaacccaagcattggcaaatgcctcattatccaaagaacactgaggtgtggagcaaaactgtgttgaatgaactgtagatggaaattctctccttcccaccgaggagccgctgcaggttgcagactcccagaatgccacactgagaagaggagggctaaaattctagagcagaatccctggggcagcccctgctgaagaagggtgcatctgttgagaatctcatcaccgcccccaccccgcccagccttcaatttagaggaatttctagttcacgcagaatgaagacaagatcacataagtgaggacgacgccccatttacaagtgtgatttggaagactgaagtggaaaccttgggaaggggcctcagagtatgtcagctggggtcagacgggaacaggagtctggctcaacacacatttattgcactcctgtttcatgccaagaaatgtaccaaaatctgggactgtgtaacacagcaaaggaaaaggctcatctccaggaagcccccttgtctaggaagagaaattgaatatcagcataaggtggtgaaaagctgtgaaggagatgagtgccaggagtccatggaggaaggattctagcctggacaggtgaggaatggctttctggaagaagcagatgccccagctgaactttaaaatgcccatgtgaaatgaactgtctgtatcttcaatcagcaaatggagtaggtggccactgtttaattctggaatggggaagtgtaaaggtgtggtgtgtgtgtttacccctacttagggattcaaaacagacagtaaagaacaaaagtctagccctttgagcattctccatgggacatttgaggtctagctggccccagagaaagagagacagaagcaaggtcatatccatttgagaaaatcaaagtgtctgctccaattgagtctggtgtccttattcctggaatagccacccctactgctctggtgctgatttacctccggcagttcagtgcttcctggaaggtgattgtggtgaatagtcatgagaatctgggaactggctcctgctctgtctatcttggagatgcatcttttgtgagaagccagggcaagaatagggaggcttaggcaatgggaagtttctgagtagataaagcagatggcagagcaaagggacagagcctggcgagaggctactccctgtagcaacctgcctaagagtgagaggaaagcaaggcatggctatgagcacgtgtagtctcagctacttgggaagctgaggctgggggatcacttgagcccaggagttcaagaccagcctaggcaacatagtgagaccatgtctctttaaaaaaaaaaaaaaaagaaaaagggagagagagaaaaaaaggtgacaattcccccaacacacataaagtacataagagttaacaaggacacagtggcagaaaatagtcagaactgagcaagcatcagtgaggaaatgttttcctgaggagacttgagaagtctcaaaaatgaaaagaaagagtatttggtgtagaaagaatggcatgagatgtgaaagctcattcatccatttattccaggtatatctttttcttcagtactttgggaggcaataggtgtaatagcaagaacattcaggttagtgggttagtgtgagtctgccatatgtatgagggttgaatacacattaggtgtggtgactgtcatatgggaggcactgcaaggtgaaggagacatgtcctcacatttggtctttgggaagcgaagtaggcagaaacccaagtgcaaagtggccatttgttttcctaatgctagataagtacctgatgtgttggaagggtggagtagaactagaaactcctggaagttcttttaagaacgaatgcaggacctcatcagcccgagtctttaaataagtcacacatttttggtcaccacattctggaagggggacaaaaagatctgtcacagacaaaatttggagatatttaaggctttgttgactttggcaaatgcttgattatagttctgtggtctgtttgttttctagagtctccatgaattgcaagggaaaacaagtggtacactggtatttctatttgataacctgttctctttggaattccggttagaaatcaaaggcttacttgagttttaatgctttcgggacttcaaatgcttttctatcttcctaataatatattaacattttctgaagacaacatatatgtggaaaacaacattatttactcaaataattttttttgaaaaatggaccaaaaaaaaaaaaaaaa

SEQ ID NO: 11 - DNA, 1865 bp, Homo Sapiens
ggtgcgcatgcgcccggagagctgaagggcgcgcggtgccgacagggagcttccttggctggcatggcggcgagcgcgctgcgaggcctgccagtggctggcggaggcgagagtagcgagagcgaggatgatggctgggagattgggtatctcgaccggacgtctcagaaattgaaaaggctattacccattgaagaaaagaaagaaaaatttaagaaagcaatgaccatcggagatgtttcattggtccaggagctcctagattctggcattagtgtagattccaactttcagtatggatggactccccttatgtatgctgctagtgttgccaatgcagagctggttcgggtccttttggacagaggtgctaatgcaagctttgagaaggataagcaaagtattttgataactgcatgttctgctcatggctcagaggaacagatcttgaagtgtgtagaactactactttcaagaaatgctgatccaaatgttgcttgtaggagacttatgaccccaatcatgtatgctgctcgagatggtcacacccaggttgttgctctccttgttgctcatggagcagaagttaatacccaggatgagaatggttacactgctttaacgtgggcagcacgtcagggtcataaaaatatagttttgaagttgcttgaacttggagctaataaaatgctacaaaccaaagatggaaagatgccaagtgagattgcaaaaagaaacaaacatcatgagatcttcaacttactttcttttactttaaatccattggaaggaaaacttcaacagctaactaaagaagacactatttgtaaaatattgacaacagattctgatagagaaaaagatcacatttttagttcatatacagcatttggagatctggaagtatttttacatggtcttggacttgaacatatgacagatttactaaaggaaagggatataacgttaagacatcttttgaccatgagggaagatgaatttacaaagaatggaattaccagtaaagaccagcagaaaattctggctgctcttaaagaactacaggtagaagagatacaatttggagagctatctgaagagacaaagttggaaatcagtggtgatgagttcctcaactttcttctcaaattaaataaacagtgtggccatttaataacagctgtacagaatgttattactgagttacctgtaaattctcaaaagataacactggaatgggcttctccccagaattttacttcagtttgtgaagaattggttaataatgttgaagatttgagtgaaaaggtctgcaaactaaaagacctaattcaaaagttgcaaaatgaacgggaaaatgatccaactcatatacaattaagggaagaagtatctacatggaatagtagaattttgaagaggacagctattaccatatgcggattcggttttcttcttttcatttgcaagctaactttccagaggaaataatccaaatatttgttttaagttatatatacacatcttattgaaccatccaaaaatcattgcttttaactgtttgcagcacaatataactttacattctttgccaatattccataggaaaaattttgacaatgtggagtttttttttaaaaagtggtttgttactttgacttataaatacaatttgttatgtttatgttaattgtgattaggtagtaaagtataagtaattttttggtttgatcgtatctaaatcacattcaaaattaaagttatatctaatatctttgacattttttaaagtgtgttcatttgtcagtgaatcgtacaggatttgtaaagtgttcataaattgagctttcttttgtttctttttt

SEQ ID NO: 12 - DNA, 2195 bp, Homo Sapiens
ggctgcaggcggggccggcgggggcgcagcggcccgggaggaagcagtaccttgaagagaaattggagagggagtcaattcctaggatagcagagagatggacaacagacagaatagatggagtttcacaatggtggccatgtgtctggaattggtgggttcttggtctcactgacttcaagaatgaagccgcacaccctcgcagtcaccccagctctgatctttgccatcacagttgctacaatcggctctttccagtttggctacaacactggggtcatcaatgctcctgagacgatcataaaggaatttatcaataaaactttgacggacaaggcaaatgcccctccctctgaggtgctgctcacgaatctctggtccttgtctgtggccatattttccgtcgggggtatgatcggctccttttccgtcggactctttgttaaccgctttggcaggcgcaattcaatgctgattgtcaacctgttggctgccactggtggctgccttatgggactgtgtaaaatagctgagtcagttgaaatgctgatcctgggccgcttggttattggcctcttctgcggactctgcacaggttttgtgcccatgtacattggagagatctcgcctactgccctgaggggtgcctttggcactctcaaccagctgggcatagttattggaattctggtggcccagatctttggtctggaactcatccttgggtctgaagagctatggccggtgctattaggctttaccatccttccagctatcctgcaaagtgcagcccttccatgttgccctgaaagtcccagatttttgctcattaacagaaaaaaagaggagaatgctacgcggatcctccagcggttgtggggcacccaggatgtatcccaagacatccaggagatgaaagatgagagtgcaaggatgtcacaagaaaagcaagtcaccgtgctggagctctttagagtgtccagctaccgacagcccatcatcatttccattgtgctccagctctctcagcagctctctgggatcaatgctgtgttctattactcaacaggaatcttcaaggatgcaggtgttcaacagcccatctatgccaccatcagcgcgggtgtggttaatactatcttcactttactttctctatttctggtggaaagggcaggaagaaggactctgcatatgataggccttggagggatggctttttgttccacgctcatgactgtttctttgttattaaagaatcactataatgggatgagctttgtctgtattggggctatcttggtctttgtggcctgttttgaaattggaccaggccccattccctggtttattgtggccgaactcttcagccagggcccccgcccagctgcgatggcagtggccggctgctccaactggacctccaacttcctagtcggattgctcttcccctctgctgcttactatttaggagcctacgtttttattatcttcaccggcttcctcattaccttcttggcctttaccttcttcaaagtccctgagacccgtggcaggacttttgaggatatcacacgggcctttgaagggcaggcacacggtgcagatagatctgggaaggacggcgtcatggggatgaacagcatcgagcctgctaaggagaccaccaccaatgtctaagtcatgcctccttccacctccctcccggcatgggaaagccacctctccctcaacaagggagagactttatcaggatgaacccaggactgcttctgaatgctgctacttgatttctttctcatcccacgcactccatgagcaccccaaggctgcagtttgttggatcttcaatggctttttaaattttatttcctggacatcctcttctgcttaggagagaccgagtgaacctaccttcatttcaggagggattggccgcttggcacatgacaactttgccagcttttcctcccttgggttctgatattgccgcactagaggatataggagaggaaaagtaaggtgcagttgccccaacctcagacttaccaggaagcagatacatatgagtgtggaagccggagggtgtttatgtaagagcaccttcctcacttccatacagctctacgcggcaaattaacttgagttttatttatcttatcctctggtttaattacataaatatttatttttta

SEQ ID NO: 13 - DNA, 531 bp, Homo Sapiens
acgagcactggagcttgcgttacttggcctcacctcacctgtgctgtccacgcctggctttgtctcacctgacgcgatatgcctctcctgcgtgggcgctgtcctgcccgccgccactaccgccgcttggccctgctcggcctgcagcccgctccccgcttcgcccactcggggcccccgcgccagcggcccctgtctgccgcggaaatggctgttggacttgtggtgttttttacgaccttcttaacaccagctgcatatgtgctaggcaacctgaagcagttcagaaggaattagatggaagatgatgttgaacagctgttaacgtccaaaaaactttcagaaaaagctgtgtttttgttaacgagcaaaattgcctagttgagttgatgcaaccattgtggtattcactttcctcatgtttatgatgaatattttgcacttttttagtactgtgcattatatagatgtatagtcaaaaatgttctgcttaagtgttaaataaaacggaaacacttattcgtgcttggt

SEQ ID NO: 14 - DNA, 2837 bp, Homo Sapiens
agtctcgcgggaagctccgttgtgggcgccccggctggtggctgagctcaggccttcaggcagaggggaggcgagggcggggcggtcacgtgagagcactgccgcggtgggttgtgggggtgctgcggcgccgtttgctttgccaaaccgacaaaagagagatgatggccaacgacgccaagcccgacgtgaagaccgtgcaggtgctgcgggacacagccaaccgcctgcggatccattccatcagggccacgtgtgcctctggttctggccagctcacgtcgtgctgcagtgcagcggaggtcgtgtctgtcctcttcttccacacgatgaagtataaacagacagacccagaacacccggacaacgaccggttcatcctctccaggggacatgctgctcctatcctctatgctgcttgggtggaggtgggtgacatcagtgaatctgacttgctgaacctgaggaaacttcacagcgacttggagagacaccctaccccccgattgccgtttgttgacgtggcaacagggtccctaggtcagggattaggtactgcatgtggaatggcttatactggcaagtaccttgacaaggccagctaccgggtgttctgccttatgggagatggcgaatcctcagaaggctctgtgtgggaggcttttgcttttgcctcccactacaacttggacaatctcgtggcggtcttcgacgtgaaccgcttgggacaaagtggccctgcaccccttgagcatggcgcagacatctaccagaattgctgtgaagcctttggatggaatacttacttagtggatggccatgatgtggaggccttgtgccaagcattttggcaagcaagtcaagtgaagaacaagcctactgctatagttgccaagaccttcaaaggtcggggtattccaaatattgaggatgcagaaaattggcatggaaagccagtgccaaaagaaagagcagatgcaattgtcaaattaattgagagtcagatacagaccaatgagaatctcataccaaaatcgcctgtggaagactcacctcaaataagcatcacagatataaaaatgacctccccacctgcttacaaagttggtgacaagatagctactcagaaaacatatggtttggctctggctaaactgggccgtgcaaatgaaagagttattgttctgagtggtgacacgatgaactccaccttttctgagatattcaggaaagaacaccctgagcgtttcatagagtgtattattgctgaacaaaacatggtaagtgtggcactaggctgtgctacacgtggtcgaaccattgcttttgctggtgcttttgctgccttttttactagagcattcgatcagctccgaatgggagccatttctcaagccaatatcaaccttattggttcccactgtggggtatccactggagaagatggagtctcccagatggccctggaggatctagccatgttccgaagcattcccaattgtactgttttctatccaagtgatgccatctcgacagagcatgctatttatctagccgccaataccaagggaatgtgcttcattcgaaccagccaaccagaaactgcagttatttataccccacaagaaaattttgagattggccaggccaaggtggtccgccacggtgtcaatgataaagtcacagtaattggagctggagttactctccatgaagccttagaagctgctgaccatctttctcaacaaggtatttctgtccgtgtcatcgacccatttaccattaaacccctggatgccgccaccatcatctccagtgcaaaagccacaggcggccgagttatcacagtggaggatcactacagggaaggtggcattggagaagctgtttgtgcagctgtctccagggagcctgatatccttgttcatcaactggcagtgtcaggagtgcctcaacgtgggaaaactagtgaattgctggatatgtttggaatcagtaccagacacattatagcagccgtaacacttactttaatgaagtaaactaggcttatttctaaaaagtcaagtctattggctttggcccaaaagcactggtatctttgtattaaattcatgtttattgtcacaaaaccattatttatacctatacagttgtactgtttcttttaaagcaaagccatttaacatctttcttcattcctaatttggaaattaaagtttacctttctgttaatctatgtataaatgttactctgagttattaatgtggattttaaaattgtaagcaatagaataggaaataaaacaactacctaatacaaatatttctgataagactacaaatatctgactgagctggggattaaagtagaggtaactgtatcttaaatgagtatgatttccttgtaagttaaaaaaattgaaatttaattgtagacttcaatagtccaagttttgaaggatgtttgagcttttgtataatgccatttatacctgcagttttacagataatgtttgactgcagttgccttggaaattcctccaaagtttgccttcatctctcctctacagtttggaggtgatggtgcagcagtggaacatctcttgatgcaccacactacttgtgttctgtgaagtgatgaaagtataactggttctagtttgcacactacacacatagttttgtgaagcttcagaaatgttttttcttttccttgtggccaaaccagtttgttaatctgattatattcatctgctaatgatactaaagttaatgtaataaagcatttaaaaatcagaaaaaaaaaaaaaaaaaaaaaaaaaaaaaaaaaaaaa

SEQ ID NO: 15 - DNA, 3848 bp, Homo Sapiens
agcgttgctgctgccttgcagtttgatctcagactgctgtgctagcaatcagcgagactccgtgggcgtaggaccctccaagccaggtgaaagagcttatgatcctaagcacttccataatagggtcagtagaatcatgatcgatgatcataatgtccccactctacgggagatggtagcattctccaaggaagtgttggagtggatggctcaagattctgaaaacatcgtagtgattcactgtaaaggaggcaaagaatagatatgttggatattttgcacaagtgaaacatagctacaactggaatctccctccaagaaaaacactgtttataaaaagattcgttatttattcgattcatggtgttggaacaggcgatggatatgatctaaaagtccaaatagtaatgaagaaaaagattgtcttttcctgtacttccttaaagaattgtcgggtatttcatgacactgaaacagacagggtaataactgatgtgttcaactgtccacctctgtatgatgatgtgaaagtgcaagcttcctcttcaagagaagagggcagcacacctcgcagggctaactggaagggggagccatccaggagacctgtgctcaactgatgggtggggagcaatagcgagaacgagggagggacctgagagtggaagcctttattgggatgtaaggtgttacctgagcaggtttcctacggggaggtctaactggtggatttaatgcaagcagtcatgagttccatggagtcatgctgtgactgagaggtggtcattgatatatccacatggtccatgcagagtacgggggtctgtagggaggttatatctagctgtcccataatgaagtagtcaccaacagaaggttgtataaggcagatactgggatcagtcacattgagaaacctggaggaggtgaactggaaactgtcaagggtgactgaaccctgcttctgatatcagaaagtccaatttatatttgaaagggatgctgaggcacaaaaaaattgtaagaattcactacaaaaatacttggctatatataagcataggtccttagtagattctgtttagcactatctaaaccagattcaaatttcagcatttaaattaaatatctatcatggaaaataaactattccttgaaaattttggtagaaacagcaagagaaagcaatagcattttcttaagcctcctcctctgtgtcttgagtgtgttattatagaatgcagagtgctacctattgaatggttataattatttgataaatatataaaggaataaaggaaggaactttgatttctttggaatgatagttcttggcatcaattttacttttaaaatatttttttttctttttaggattttcctaaatactatcacaactacccttttttcttctggtttaacacatctttaatacaaaataacgggtatggatataatatcaacccatagaaacaacctaatcttcaatgtctatgtataagatgtaatggcaagtcttttgctggttgtcataagcttaatttatagaaaacaaaaaatccttgagccaccattgttcattgccttactccttttacgttggctattttaaaaatacagttgttcttgagacccccagttgcagtatcctcaaggtccatgccataggactgtgttatgagctcaaaagtattataatcagatcttaagtgtggaagtaaattcctcccagagaagttcaatatgaatctgctcagtaccttcaacatgtcaggtcctcagtaggtgctgatttaccaatgacgaaccaccaccaaattttgtgctaaagtaagggaggacctagggaagcttcagctagctgaaaagctgactgacacacttatatctaggagaagttacaagacacagtaagtattaagaaatacagctaaaaaatcattaaaattggtagtctcccatttaaacatgggtttctaataactgaattgggaaaactttcttaaaaactattaattggaggctgggtgtggtggctcatgcctgtaatcctagcactttgggaggctgaggcgggcggatcacctaaggttggaagttcgagactagcctggccaacatggtaaaactccgtctctactaaaaatacaaaaatcagccaggcgtggtggcacatgcctgtaatcccagctactcaggaggctgagccagtagaatcgcttgaacccaggaggcagattgcagtgagccgagatcgcaccactacactccagcctgggcgacagagtgagactctgtctaaagaaaaaagcaaaaaaacagaacaactattacttggatttggagattattgttcccagaaaaccttctgccatatttggaaacttatttctcagtctagaagttctccactttaagtagcatttgttctgtgctggtgaaaaactgagatttttttgtattaaccatactcttcaatacaaaaggagaaaatatttttaaaatgcttcaggtcacagttgaggcagttgctatgattgcatgtggcatgaattggtagttattgttacaaccagttctagtcttttcttcaaatctgagctggatctaataactccttaagtccagcaaggcaacagtaaattaaacctctggtctacacacttgcaatacatacacatttaatagattttgatagagtgaactttggattggatggaaattttttaaaaatttgtttcttggatgcatacaaacaataagctttgactcctaacatgagcaaagtccctcaattgtgagagctgggtggagcttcatttgttgctgctcctcaaattgattcttggtaaaggatacagatttttcctttgaaacaccatgttcattttggggaagcaataagttagatcacctttattttcacttttatataaatttctaaagatttctgtaatatttaaatttatatactattggtaaagctgtttttcttagttgtgaaattgttgtttagccaaaaatgccaacttctgtcttttagaacactaggcataaatgggttaaccaatttatgcctagtgttccattattggaatgctaagcatgtgggatttatttatatcctactgctcaaggtcatcgccaagggctgtttgcaaaaattcaaaaaattgcaacctcaggcataaattaaaagagatatagtattttattattgggttttgatacatgtctaatcagactgatttctgtcacatatagaaatttagatactgtattaaacctggatgtcattaattccataaaaagcaacgttaaaagaatcagtagcatgtgttactgatgtgttgctgaagattaagatatttttaagtctcaccgaaaaggtagaaggagccaactgagacacaaaaaggggctgaggttctattcatggtgagcaagtctttttttttgtttgtttcttcaagctctaacaagggtgcctactacatggcttttcagttagccccaaaataagatgtaacaatttttttttctattcttaggctttatctacaaagaaatgaattggataatcttcataaacaaaaaacatggaaaatttatcaaccagaatatgcagtagagatatattttaatgagaaatgacttaagttatgttgtaactggtagctgattaagtatagttccctgcaccccttctgggaaagaattatgttctttctaaccctgccacatagttatatgttctaaatcttccttgctggtacatctatattgatatatgtatacacatgttctttataaatctattaaatatatacagaaaaaaaaaaaaaaaa

SEQ ID NO: 16 - DNA, 1049 bp, Homo Sapiens
gttagacacaggacctgctgggccacagaaaggaggctctgggtagacgcactagattactggataaatcacttcaatttcccaatgaattttatattgtttatttttatacctggagttttttccttaaaaagtagcactttgaagcctactattgaagcattgcctaatgtgctacctttaaatgaagatgttaataagcaggaagaaaagaatgaagatcatactcccaattatgctcctgctaatgagaaaaatggcaattattataaagatataaaacaatatgtgttcacaacacaaaatccaaatggcactgagtctgaaatatctgtgagagccacaactgacctgaattttgctctaaaaaacgataaaactgtcaatgcaactacatatgaaaaatccaccattgaagaagaaacaactactagcgaaccctctcataaaaatattcaaagatcaaccccaaacgtgcctgcattttggacaatgttagctaaagctataaatggaacagcagtggtcatggatgataaagatcaattatttcacccaattccagagtctgatgtgaatgctacacagggagaaaatcagccagatctagaggatctgaagatcaaaataatgctgggaatctcgttgatgaccctcctcctctttgtggtcctcttggcattctgtagtgctacactgtacaaactgaggcatctgagttataaaagttgtgagagtcagtactctgtcaacccagagctggccacgatgtcttactttcatccatcagaaggtgtttcagatacatccttttccaagagtgcagagagcagcacatttttgggtaccacttcttcagatatgagaagatcaggcacaagaacatcagaatctaagataatgacggatatcatttccataggctcagataatgagatgcatgaaaacgatgagtcggttacccggtgaagaaatcaaggaacccggtgaagaaatcttattgatgaataaataactttaattattttgtcatcaaaaaaaaaaaaaaa

SEQ ID NO: 17 - DNA, 3591 bp, Homo Sapiens
ttactgggcgtatggcgtacagacacgaggccggcgcccgggaggcggtgttcatccgcccgggaaaagagcgcctgttgctcgctgcccgcgtgtccctggctctctcgggaacccagcgccgaaggcgaggtgggcgcgggccgaaggaggtcctgggaggtcggcggcgcggagggatctccgcgggagccgttggggctgttggcctcgggctgaggtgcaaggaccaggactagggcgagggcagcggtccaagaaatagaaaacaatgactgggagagcccgagccagagccagaggaagggcccgcggtcaggagacagcgcagctggtgggctccactgccagtcagcaacctggttatattcagcctaggcctcagccgccaccagcagagggggaattatttggccgtggacggcagagaggaacagcaggaggaacagccaagtcacaaggactccagatatctgctggatttcaggagttatcgttagcagagagaggaggtcgtcgtagagattttcatgatcttggtgtgaatacaaggcagaacctagaccatgttaaagaatcaaaaacaggttcttcaggcattatagtaaggttaagcactaaccatttccggctgacatcccgtccccagtgggccttatatcagtatcacattgactataacccactgatggaagccagaagactccgttcagctcttctttttcaacacgaagatctaattggaaagtgtcatgcttttgatggaacgatattatttttacctaaaagactacagcaaaaggttactgaagtttttagtaagacccggaatggagaggatgtgaggataacgatcactttaacaaatgaacttccacctacatcaccaacttgtttgcagttctataatattattttcaggaggcttttgaaaatcatgaatttgcaacaaattggacgaaattattataacccaaatgacccaattgatattccaagtcacaggttggtgatttggcctggcttcactacttccatccttcagtatgaaaacagcatcatgctctgcactgacgttagccataaagtccttcgaagtgagactgttttggatttcatgttcaacttttatcatcagacagaagaacataaatttcaagaacaagtttccaaagaactaataggtttagttgttcttaccaagtataacaataagacatacagagtggatgatattgactgggaccagaatcccaagagcacctttaagaaagccgacggctctgaagtcagcttcttagaatactacaggaagcaatacaaccaagagatcaccgacttgaagcagcctgtcttggtcagccagcccaagagaaggcggggccctggggggacactgccagggcctgccatgctcattcctgagctctgctatcttacaggtctaactgataaaatgcgtaatgattttaacgtgatgaaagacttagccgttcatacaagactaactccagagcaaaggcagcgtgaagtgggacgactcattgattacattcataaaaacgataatgttcaaagggagcttcgagactggggtttgagctttgattccaacttactgtccttctcaggaagaattttgcaaacagaaaagattcaccaaggtggaaaaacatttgattacaatccacaatttgcagattggtccaaagaaacaagaggtgcaccattaattagtgttaagccactagataactggctgttgatctatacgcgaagaaattatgaagcagccaattcattgatacaaaatctatttaaagttacaccagccatgggcatgcaaatgagaaaagcaataatgattgaagtggatgacagaactgaagcctacttaagagtcttacagcaaaaggtcacagcagacacccagatagttgtctgtctgttgtcaagtaatcggaaggacaaatacgatgctattaaaaaatacctgtgtacagattgccctaccccaagtcagtgtgtggtggcccgaaccttaggcaaacagcaaactgtcatggccattgctacaaagattgccctacagatgaactgcaagatgggaggagagctctggagggtggacatccccctgaagctcgtgatgatcgttggcatcgattgttaccatgacatgacagctgggcggaggtcaatcgcaggatttgttgccagcatcaatgaagggatgacccgctggttctcacgctgcatatttcaggatagaggacaggagctggtagatgggctcaaagtctgcctgcaagcggctctgagggcttggaatagctgcaatgagtacatgcccagccggatcatcgtgtaccgcgatggcgtaggagacggccagctgaaaacactggtgaactacgaagtgccacagtttttggattgtctaaaatccattggtagaggttacaaccctagactaacggtaattgtggtgaagaaaagagtgaacaccagattttttgctcagtctggaggaagacttcagaatccacttcctggaacagttattgatgtagaggttaccagaccagaatggtatgacttttttatcgtgagccaggctgtgagaagtggtagtgtttctcccacacattacaatgtcatctatgacaacagcggcctgaagccagaccacatacagcgcttgacctacaagctgtgccacatctattacaactggccaggtgtcattcgtgttcctgctccttgccagtacgcccacaagctggcttttcttgttggccagagtattcacagagagccaaatctgtcactgtcaaaccgcctttactacctctaacctgcagaagacgatgcagccgcttttctttttgaaatgactttgggatttttttaagcttttatttactttttttttaactgttatctttctggatgaaacttgggaaggggattaggagatctagcattttatttctagcattgctattcaccggcttccttattttatacgtaaaaattaagattttatattttatcttcttgtttctcatagatattttgtgagcatttttttgtttattttgaagaaatgtggataagatacttggtagtataaaacagactctctgagagtatttgaaatgtgtttggagatttacttaaacgtactttcaggagtgagcaagtcctacttataaacctatattaactttatttttgagatacctgttttgaatttaaaggagataagaggcgtaaagtaggatgctcactacaaccataggtggggtttcagctcatatcttaaagataaaaggtactattatataacctatacacaagatacaggagaaaatatgcttgatttttatttggcaggggggctaggttgtatgggagtaaaaaaaacattgaaaatttttaaattgtccaaagaaacattttaagactctttaacaaaaaaggccatgagtaaatctctatattaacattactatttattttgttttggaactgggacatgattctatttgttataaaataaaattgatgtgattgtcaccttatttg

SEQ ID NO: 18 - DNA, 819 bp, Homo Sapiens
gtgatgctccctgggcctcctgaccgcgccctcgcctgggaggcggggcgggccgggttctctctgtgacgtcacaaaggccccgccatgcctctggctttgacccttctgctgctctcgggcttgggcgcccccggaggctggggctgcctgcagtgcgaccccttggtgctggaggccctgggtcacctgcgctccgccctcatccccagtcgcttccagttggagcagctgcaggcgcgcgccggggccgtgctgatgggcatggaggggcctttcttccgggactacgcgctgaacgtgtttgtggggaaagtggagacaaatcaactggaccttgtggcgtcctttgtcaagaaccaaacgcagcacttaatgggtaactctctgaaagatgagcctctgctggaagagctggtgaccctcagggcgaatgtgatcaaggaattcaagaaagttttaatttcatatgaattaaaagcctgcaaccccaaactttgccgcttgctaaaagaagaggtgttggactgtttacattgccagaggatcactcccaagtgtatccacaaaaagtactgctttgtcgaccggcaaccccgcgtggccctgcagtaccagatggacagcaaatacccgaggaaccaggcgctgttgggcatcctcatttctgtgtctctggctgtctttgtcttcgtggtcatcgtggtctcggcttgtacatacagacaaaaccgaaaactcctgctgcagtaggacggtggtttgggggtaaggagaaaggaaaataaatttaataaaattggtgacaaatccaaaaaa

SEQ ID NO: 19 - DNA, 5120 bp, Homo Sapiens
ggactcgcactcggcggttgttccagaagaaagagacagcgatggcggcagaggcttcgaagactgggccttctaggtcttcctaccagcgaatggggaggaagagtcagccctggggtgccgctgaaatccagtgcaccaggtgtggaaggagggtatccagatcatccggtcaccattgtgaacttcaatgtggacatgctttttgtgaactatgcttgttaatgactgaagaatgcaccacaattatatgccctgattgtgaggttgctacagctgtaaatactagacaacgctactacccaatggctggatatattaaggaagactccataatggaaaaactgcagcctaagacgataaagaattgttctcaggactttaagaagactgctgatcagctaactactggtttagaacgttcagcctccacagacaagactcttttgaactcatcagctgtaatgttggacactaatactgcagaagaaattgatgaagcattgaatacagcacaccatagtttcgaacagttaagcattgctggaaaagcacttgaacacatgcagaagcaaacgatagaggaaagagaaagagttatagaagttgtggagaaacagtttgaccaacttttggctttttttgattccaggaaaaagaacctgtgtgaagaatttgcaagaactactgatgattatctatcaaatttaataaaggctaaaagctacattgaagagaaaaaaaataatttgaatgcagctatgaacatagcaagagcattacaattatcgccttctctaagaacatactgtgacctgaatcagattatccggactttgcagttaacttcagatagtgaattagcacaagttagttctccacaactaaggaaccctcccaggttgagtgtgaattgcagtgagatcatctgtatgttcaacaatatgggaaagattgaatttagggactcaacaaaatgttatccccaagaaaatgaaattagacagaatgttcaaaagaaatataataacaaaaaggaactttcttgttacgatacatacccaccgctagaaaagaaaaaggttgacatgtctgtcctaaccagtgaagcaccaccacctcctttgcaacctgagacaaatgatgtacatttagaagcaaaaaacttccagccacagaaagacgttgcaacagcatcccctaaaaccattgctgtgttacctcagatgggatctagccctgatgtgataattgaagaaattattgaagacaacgtggaaagttctgcagagctagtttttgtaagccatgtaatagatccttgccatttctacattcggaagtattcacaaataaaagacgccaaagtactggagaagaaggtgaatgaattttgcaataggagttcacaccttgatccttcagacattttggaactaggtgcaagaatatttgtcagcagtattaaaaatggaatgtggtgtcgaggaactatcacagaattaattccaatagagggtagaaataccagaaaaccttgtagtccaaccagattatttgtccatgaagttgcactaatacaaatattcatggtagattttggaaattctgaagtcctgattgtcactggagttgttgatacccatgtgagaccagaacactctgctaagcaacatattgcactaaatgatttatgtctggttctaaggaaatctgaaccatatactgaagggctgctaaaagacatccagccattagcacaaccatgctcattgaaagacattgttccacagaattcaaatgaaggctgggaagaggaagctaaagtggaatttttgaaaatggtaaataacaaggctgtttcaatgaaagtttttagagaagaagatggtgtgcttattgtagatctgcaaaaaccaccaccgaataaaataagcagtgatatgcctgtgtctcttagagatgcgctagtttttatggaactagcaaagtttaagtcacaatcactaagaagtcactttgaaaaaaatactactttacactatcatccacctattttgcctaaagaaatgacagatgtttcagtaacggtttgtcatataaatagtcctggagatttctatcttcagttgatagagggcctggatattttatttctattaaagacaatcgaggaattctataaaagtgaagatggagaaaatctggaaatcctctgtccagttcaagatcaagcctgtgtagctaaatttgaagatggaatttggtaccgagcaaaagttatcggattgcctggacatcaggaagttgaagttaaatatgtggactttggtaatactgcaaaaataacaatcaaagacgtgcgtaaaataaaggatgagtttctgaatgccccagagaaggcaattaaatgtaagttggcctatattgaaccatataaaaggacaatgcagtggtccaaagaagctaaagaaaaatttgaagaaaaggctcaagataaatttatgacatgttcagttatcaaaattctggaagataatgtgctcttagttgagcttttcgattctcttggtgctcctgaaatgactactactagtattaatgaccagctagttaaagagggcctagcatcttatgaaataggatacatcctcaaagataattctcaaaagcatattgaagtttgggatccttctccagaagaaattatttcaaatgaagtacacaacttaaatcctgtgtctgcaaaatctctacctaatgagaattttcagtcactttataataaggaattgcctgtgcatatctgtaatgtaatatctcctgagaagatttatgttcagtggttgttaactgaaaacttacttaatagtttagaagaaaagatgatagctgcttatgaaaactcaaaatgggaacctgttaaatgggaaaatgatatgcactgtgctgttaagatccaagataaaaatcagtggcgaagaggccagatcatcagaatggttacagacacattggtagaggtcttgctgtatgatgtgggtgttgaactagtagtgaatgttgactgtttaagaaaacttgaagaaaatctaaagacaatgggaagactctctttggaatgttctctggttgacataagaccagctggtgggagtgacaagtggacagcaacagcttgtgactgtctttcattgtacctgactggagctgtagcaactataatcttacaggtggatagtgaggaaaacaacacaacatggccattacctgtgaaaattttctgcagagatgaaaaaggagagcgtgttgatgtttctaaatatttgattaaaaagggtttggctttgagagaaaggagaattaataacttagataacagccattcattatctgagaagtctctggaagtccccctggaacaggaagattcagtagttactaactgtattaaaactaactttgaccctgacaagaaaactgctgacataatcagtgaacagaaagtgtctgaatttcaggagaaaattctagaaccaagaaccactagagggtataagccaccagctattcctaacatgaacgtatttgaggcaacagtcagctgtgttggtgatgatggaactatatttgtagtacctaaactatcagaatttgagctaataaaaatgacaaatgaaattcaaagtaatttaaaatgccttggtcttttggagccttatttctggaaaaaaggagaagcatgtgcagtaagaggatccgatactctgtggtatcgtggcaaggtgatggaggttgtaggtggcgctgtcagagtacaatatttagatcatggattcactgaaaagattccgcagtgccatctttaccctattttgctgtatcctgatataccccagttttgtattccttgtcagctccataataccacacctgttgggaatgtctggcaaccagatgcaatagaagttcttcaacaactgctttcaaagagacaggtggacattcacattatggagttacctaaaaatccatgggagaaattgtctattcacctctattttgatggaatgtcactttcttattttatggcatactataaatactgtacttctgaacatactgaggagatgttgaaagaaaaaccaagatcagatcatgataaaaagtatgaagaggaacaatgggaaataaggtttgaggaattgctttcggctgaaacagacactcctcttttaccaccatatttgtcttcatctctgccttccccaggagaactctatgctgttcaagttaagcacgttgtctcacctaatgaagtgtatatttgccttgattctatagaaacttctaaccagtctaaccagcatagtgacacagatgatagtggagtcagcggggaatcagaatccgagagccttgatgaagcactgcagagggttaataagaaggtagaggcgcttcctcctctgacggattttagaacagaaatgccttgccttgcagaatatgatgatggcttatggtatagagcgaagattgttgccattaaagaatttaatcctttatctatcttagtacaatttgttgattatggatcaactgcaaagctgacattaaacagactgtgccaaattccttctcatcttatgcggtatccagctcgagccataaaggttctcttggcagggtttaaacctcccttaagggatctaggggagacaagaataccatattgtcccaaatggagcatggaggcactgtgggctatgatagactgtcttcaaggaaaacaactctatgctgtgtccatggctccagcaccagaacagatagtgacattatatgacgatgaacagcatccagttcatatgccgttggtagaaatggggcttgcagataaagatgaataagtgcctaagtgtatacagtgagagcatctatagaagcctagaagaattctgttatgtttagactatgtcttatctttagactatttcaggcttaattttcctaacttgttcagcactagtgctttacctctcatttttaattgaactgttaggaattgtgtggggaaaaaaagtaaataaatgttcgcttccaaaaaaaaaaaaaaa

SEQ ID NO: 20 - DNA, 1738 bp, Homo Sapiens
gatgtcatcaggctttgtgagggggactgtactgccctttgagtactactgtgtggccccgcaacccagcacaaccaggtatctgcttggaacccagccaccataaagcctgctagctaaaaaaaattttacatctctcagttcattcggcacagacccctgcctcattcagctgtgactctgcttggaaaattcatcagttacaaagcagccaatgcaattatctcaagggaaattgaaaaatggacctttgaaaatgctagatttacaatgagaaatgccataattcaaggtttattctatgggtccttgacatttgggatctggacagctctgttattcatatatttgcaccataatcatgtgagcagctggcagaagaaaagccaggagcctctgtcagcttggtcccctggaaaaaaagtgcatcagcaaattatctatggctcagagcaaataccaaaacctcatgtaatagtcaaaaggactgatgaagataaagcaaagtctatgttaggtacagattttaaccatacaaacccagaacttcataaagaacttttaaaatatggatttaatgtgattatcagtagaagcttgggcatcgaaagagaagtgccagataccaggagtaaaatgtgtcttcaaaaacattacccagcccgcctcccgactgccagcattgtcatttgcttctataatgaagaatgtaatgccttgtttcagaccatgtccagtgtcacgaacctcacgccacactattttcttgaagaaattattttggtagatgacatgagcaaagttgatgatttgaaagaaaaactagactatcacctggaaacttttcggggaaaggttaaaataataagaaacaaaaagagagaggggctgattcgagcaaggctgattggagcttctcatgcttcaggggatgttctggtgttcctggacagccactgtgaggtgaacagagtatggctggagcccctgctgcatgccattgccaaggaccccaaaatggtggtgtgccccctgatagatgtcattgatgatagaactctggagtataagccctctcctcttgtaaggggaacttttgattggaacctacaatttaaatgggataatgttttctcttatgagatggatggaccagaaggatctactaaaccaatccggtcacctgcaatgtctggaggaatttttgctatacgtcggcattattttaatgaaattggacagtatgacaaggatatggatttttggggaagagaaaatttggaactttcactaaggatctggatgtgtggaggccaactctttataatcccctgctctcgagtaggacatatcagtaagaaacaaactggaaaaccttctacaatcatcagtgctatgacacataactacctaagactggtgcacgtttggctggatgaatataaggagcagttttttcttcgaaagcctggtctgaaatatgtcacctacggaaatattcgcgagcgtgttgagttaaggaaacgactgggttgcaagtcatttcagtggtatttggataatgtcttcccagagttggaggcatctgtgaacagcctgtgaaaggaaaacaaatcactttcattaataaagggttaaaagtctcctagtcattcaacatagtgtcacaagagtgtaagtttggaacatcgtggaattacgtgaaatgcaattaaaaaaatatgaccagacgtgaaaa

SEQ ID NO: 21 - DNA, 731 bp, Homo Sapiens
gagaactcttgagaccgggagcccagctgcccaccctctggacattcacccagccaggtggtctcgtcacctcagaggctccgccagactcctgcccaggccaggactgaggcaagcctcaaggcacttctaggacctgcctcttctcaccaagatgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagcccccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctgagggcgcaggtgcggggcagtgaacttcagaccccaaaggagtcagagcatgcggggcgggggcggggggcggggacgtagggctaagggagggggcgctggagcttccaacccgaggcaataaaagaaatgttgcgtaactcaaaaaaaaaaaaaaaaaa

SEQ ID NO: 22 - DNA, 1660 bp, Homo Sapiens
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 23 - DNA, 1949 bp, Homo Sapiens
aataaaggggtctgagccggtcgcctgagcctgaaaagtgctgtcacgtcagcggaaggaggcgtcccagatcttctcagctgtcttggtgccagccttcctagtcttcctacccacactcctacctgctgtcacaggccacagccatcatgcctcggggtcacaagagtaagctccgtacctgtgagaaacgccaagagaccaatggtcagccacagggtctcacgggtccccaggccactgcagagaagcaggaagagtcccactcttcctcatcctcttctcgcgcttgtctgggtgattgtcgtaggtcttctgatgcctccattcctcaggagtctcagggagtgtcacccactgggtctcctgatgcagttgtttcatattcaaaatccgatgtggctgccaacggccaagatgagaaaagtccaagcacctcccgtgatgcctccgttcctcaggagtctcagggagcttcacccactggctctcctgatgcaggtgtttcaggctcaaaatatgatgtggctgccaacggccaagatgagaaaagtccaagcacttcccatgatgtctccgttcctcaggagtctcagggagcttcacccactggctcgcctgatgcaggtgtttcaggctcaaaatatgatgtggctgccgagggtgaagatgaggaaagtgtaagcgcctcacagaaagccatcatttttaagcgcttaagcaaagatgctgtaaagaagaaggcgtgcacgttggcgcaattcctgcagaagaagtttgagaagaaagagtccattttgaaggcagacatgctgaagtgtgtccgcagagagtacaagccctacttccctcagatcctcaacagaacctcccaacatttggtggtggcctttggcgttgaattgaaagaaatggattccagcggcgagtcctacacccttgtcagcaagctaggcctccccagtgaaggaattctgagtggtgataatgcgctgccgaagtcgggtctcctgatgtcgctcctggttgtgatcttcatgaacggcaactgtgccactgaagaggaggtctgggagttcctgggtctgttggggatatatgatgggatcctgcattcaatctatggggatgctcggaagatcattactgaagatttggtgcaagataagtacgtggtttaccggcaggtgtgcaacagtgatcctccatgctatgagttcctgtggggtccacgagcctatgctgaaaccaccaagatgagagtcctgcgtgttttggccgacagcagtaacaccagtcccggtttatacccacatctgtatgaagacgctttgatagatgaggtagagagagcattgagactgagagcttaaggcagggctggcactatttccttggccagggtaccttatggggccatatcctacagatcctcccatttctagggaggtctgaagtagaattttcactttatgttagaagagagtagtgagctttctaagtagtgcagtatagtagaggctggagggaacaagatatgtatctttcttttgttacacatgagtaacttgcagatttatgttttatctctgtcagttatcaacattgttcctgttaagtgaaggtttattttgcttcagattatacaattatcaataacatagctctcacattcatggctgtttaaccaatctgaaagttacggtttgggaattaataaaacaaagtcatacaacacattttctttgtaattgagaactagataacatggtaacagagaattgattttcatatgaatcttaactccacagtaaaatagttgacatcataatatgaagagaaagaaaaggaaaaacagaaatgtaaaagttgtttaattcttggtttgcctaattcgttttcctatttcttttcatacaaataaaggatacctggatttatttaggtta

SEQ ID NO: 24 - protein, 79 aa, Homo Sapiens
MCCNYYGNSCGYGSGCGCGYGSGSGCGCGYGTGYGCGYGCGFGSHYGCGYGTGYGCGYGSGSGYCGYRPFCFRRCYSSC

SEQ ID NO: 25 - protein, 70 aa, Homo Sapiens
MKQKQEVMFQSRGRLSLYIQMSSVYSAKLGPVGGICGQKQKPSFFFFKAQSQDARPLAPAACISKIAKAG

SEQ ID NO: 26 - protein, 265 aa, Homo Sapiens
MEALPPVRSSLLGILLQVTRLSVLLVQNRDHLYNFLLLKINLFNHWVSGLAQEARGSCNWQAHLPLGAAACPLGQALWAGLALIQVPVWLVLQGPRLMWAGMWGSTKGLGLALLSAWEQLGLSVAIWTDLFLSCLHGLMLVALLLVVVTWRVCQKSHCFRLGRQLSKALQVNCVVRKLLVQLRRLYWWVETMTALTSWHLAYLITWTTCLASHLLQAAFEHTTQLAEAQEVEPQEVSGSSLLPSLSASSDSESGTVLPEQETPRE

SEQ ID NO: 27 - protein, 533 aa, Homo Sapiens
MNEENIDGTNGCSKVRTGIQNEAALLALMEKTGYNMVQENGQRKFGGPPPGWEGPPPPRGCEVFVGKIPRDMYEDELVPVFERAGKIYEFRLMMEFSGENRGYAFVMYTTKEEAQLAIRILNNYEIRPGKFIGVCVSLDNCRLFIGAIPKEKKKEEILDEMKKVTEGVVDVIVYPSATDKTKNRGFAFVEYESHRAAAMARRKLIPGTFQLWGHTIQVDWADPEKEVDEETMQRVKVLYVRNLMISTTEETIKAEFNKFKPGAVERVKKLRDYAFVHFFNREDAVAAMSVMNGKCIDGASIEVTLAKPVNKENTWRQHLNGQISPNSENLIVFANKEESHPKTLGKLPTLPARLNGQHSPSPPEVERCTYPFYPGTKLTPISMYSLKSNHFNSAVMHLDYYCNKNNWAPPEYYLYSTTSQDGKVLLVYKIVIPAIANGSQSYFMPDKLCTTLEDAKELAAQFTLLHLDYNFHRSSINSLSPVSATLSSGTPSVLPYTSRPYSYPGYPLSPTISLANGSHVGQRLCISNQASFF

SEQ ID NO: 28 - protein, 123 aa, Homo Sapiens
MPDASLGSLGITWCFLESPLEVSSGRFGLARLLGSQDHGDDPAERGRTATDAWGPSRWGQSPGNGGGYCDASPPSALAPGDRAWALPASPSSGAPASQHCCLEKAGTRTKASPVWGRDGNTWN

SEQ ID NO: 29 - protein, 638 aa, Homo Sapiens
MVVSADPLSSERAEMNILEINQELRSQLAESNQQFRDLKEKFLITQATAYSLANQLKKYKCEEYKDIIDSVLRDELQSMEKLAEKLRQAEELRQYKALVHSQAKELTQLREKLREGRDASRWLNKHLKTLLTPDDPDKSQGQDLREQLAEGHRLAEHLVHKLSPENDEDEDEDEDDKDEEVEKVQESPAPREVQKTEEKEVPQDSLEECAVTCSNSHNPSNSNQPHRSTKITFKEHEVDSALVVESEHPHDEEEEALNIPPENQNDHEEEEGKAPVPPRHHDKSNSYRHREVSFLALDEQKVCSAQDVARDYSNPKWDETSLGFLEKQSDLEEVKGQETVAPRLSRGPLRVDKHEIPQESLDGCCLTPSILPDLTPSYHPYWSTLYSFEDKQVSLALVDKIKKDQEEIEDQSPPCPRLSQELPEVKEQEVPEDSVNEVYLTPSVHHDVSDCHQPYSSTLSSLEDQLACSALDVASPTEAACPQGTWSGDLSHHQSEVQVSQAQLEPSTLVPSCLRLQLDQGFHCGNGLAQRGLSSTTCSFSANADSGNQWPFQELVLEPSLGMKNPPQLEDDALEGSASNTQGRQVTGRIRASLVLILKTIRRRLPFSKWRLAFRFAGPHAESAEIPNTAGRTQRMAG

SEQ ID NO: 30 - protein, 909 aa, Homo Sapiens
MAARPLPVSPARALLLALAGALLAPCEARGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 31 - protein, 123 aa, Homo Sapiens
MVDPVGELAAKRSGLTVEDVRANVPEELSIWIDPFGVSYQIGEKGAVKVLYLDDSDGCGAPELDMKIKSSFTPDDQMLFLLGSQDSSLSNSPSLCFAHHPAPPSSSASPSPSPSPWTPLLPPN

SEQ ID NO: 32 - protein, 753 aa, Homo Sapiens [sequence omitted]

SEQ ID NO: 33 - protein, 113 aa, Homo Sapiens
MEIKKETMLDDTGADNKRREPKHWQMRHYPKNTGPEYVCQLGSDRNRSLTQHTFIALLLHAKKCTKIWDCVTQRRKRLISRRPPCLGRETEHQHKVVKSYEGGECHESTEEGF

SEQ ID NO: 34 - protein, 475 aa, Homo Sapiens
MAASALRGLPVAGGGESSESEDDGWEIGYLDRTSQKLKRLLPIEEKKEKFKKAMTIGDVSLVQELLDSGISVDSNFQYGWTPLMYAASVANAELVRVLLDRGANASFEKDKQSILITACSAHGSEEQILKCVELLLSRNADPNVACRRLMTPIMYAARDGHTQVVALLVAHGAEVNTQDENGYTALTWAARQGHKNIVLKLLELGANKMLQTKDGKMPSEIAKRNKHHEIFNLLSFTLNPLEGKLQQLTKEDTICKILTTDSDREKDHIFSSYTAFGDLEVFLHGLGLEHMTDLLKERDITLRHLLTMREDEFTKNGITSKDQQKILAALKELQVEEIQFGELSEETKLEISGDEFLNFLLKLNKQCGHLITAVQNVITELPVNSQKITLEWASPQNFTSVCEELVNNVEDLSEKVCKLKDLIQKLQNERENDPTHIQLREEVSTWNSRILKRTAITICGFGFLLFICKLTFQRK

SEQ ID NO: 35 - protein, 520 aa, Homo Sapiens
MEFHNGGHVSGIGGFLVSLTSRMKPHTLAVTPALIFAITVATIGSFQFGYNTGVINAPETIIKEFINKTLTDKANAPPSEVLLTNLWSLSVAIFSVGGMIGSFSVGLFVNRFGRRNSMLIVNLLAATGGCLMGLCKIAESVEMLILGRLVIGLFCGLCTGFVPMYIGEISPTALRGAFGTLNQLGIVIGILVAQIFGLELILGSEELWPVLLGFTILPAILQSAALPCCPESPRFLLINRKKEENATRILQRLWGTQDVSQDIQEMKDESARMSQEKQVTVLELFRVSSYRQPIIISIVLQLSQQLSGINAVFYYSTGIFKDAGVQQPIYATISAGVVNTIFTLLSLFLVERAGRRTLHMIGLGGMAFCSTLMTVSLLLKNHYNGMSFVCIGAILVFVACFEIGPGPIPWFIVAELFSQGPRPAAMAVAGCSNWTSNFLVGLLFPSAAYYLGAYVFIIFTGFLITFLAFTFFKVPETRGRTFEDITRAFEGQAHGADRSGKDGVMGMNSIEPAKETTTNV

SEQ ID NO: 36 - protein, 72 aa, Homo Sapiens
MPLLRGRCPARRHYRRLALLGLQPAPRFAHSGPPRQRPLSAAEMAVGLVVFFTTFLTPAAYVLGNLKQFRRN

SEQ ID NO: 37 - protein, 626 aa, Homo Sapiens
MMANDAKPDVKTVQVLRDTANRLRIHSIRATCASGSGQLTSCCSAAEVVSVLFFHTMKYKQTDPEHPDNDRFILSRGHAAPILYAAWVEVGDISESDLLNLRKLHSDLERHPTPRLPFVDVATGSLGQGLGTACGMAYTGKYLDKASYRVFCLMGDGESSEGSVWEAFAFASHYNLDNLVAVFDVNRLGQSGPAPLEHGADIYQNCCEAFGWNTYLVDGHDVEALCQAFWQASQVKNKPTAIVAKTFKGRGIPNIEDAENWHGKPVPKERADAIVKLIESQIQTNENLIPKSPVEDSPQISITDIKMTSPPAYKVGDKIATQKTYGLALAKLGRANERVIVLSGDTMNSTFSEIFRKEHPERFIECIIAEQNMVSVALGCATRGRTIAFAGAFAAFFTRAFDQLRMGAISQANINLIGSHCGVSTGEDGVSQMALEDLAMFRSIPNCTVFYPSDAISTEHAIYLAANTKGMCFIRTSQPETAVIYTPQENFEIGQAKVVRHGVNDKVTVIGAGVTLHEALEAADHLSQQGISVRVIDPFTIKPLDAATIISSAKATGGRVITVEDHYREGGIGEAVCAAVSREPDILVHQLAVSGVPQRGKTSELLDMFGISTRHIIAAVTLTLMK

SEQ ID NO: 38 - protein, 522 aa, Homo Sapiens [sequence omitted]

SEQ ID NO: 39 - protein, 294 aa, Homo Sapiens
MNFILFIFIPGVFSLKSSTLKPTIEALPNVLPLNEDVNKQEEKNEDHTPNYAPANEKNGNYYKDIKQYVFTTQNPNGTESEISVRATTDLNFALKNDKTVNATTYEKSTIEEETTTSEPSHKNIQRSTPNVPAFWTMLAKAINGTAVVMDDKDQLFHPIPESDVNATQGENQPDLEDLKIKIMLGISLMTLLLFVVLLAFCSATLYKLRHLSYKSCESQYSVNPELATMSYFHPSEGVSDTSFSKSAESSTFLGTTSSDMRRSGTRTSESKIMTDIISIGSDNEMHENDESVTR

SEQ ID NO: 40 - protein, 861 aa, Homo Sapiens
MTGRARARARGRARGQETAQLVGSTASQQPGYIQPRPQPPPAEGELFGRGRQRGTAGGTAKSQGLQISAGFQELSLAERGGRRRDFHDLGVNTRQNLDHVKESKTGSSGIIVRLSTNHFRLTSRPQWALYQYHIDYNPLMEARRLRSALLFQHEDLIGKCHAFDGTILFLPKRLQQKVTEVFSKTRNGEDVRITITLTNELPPTSPTCLQFYNIIFRRLLKIMNLQQIGRNYYNPNDPIDIPSHRLVIWPGFTTSILQYENSIMLCTDVSHKVLRSETVLDFMFNFYHQTEEHKFQEQVSKELIGLVVLTKYNNKTYRVDDIDWDQNPKSTFKKADGSEVSFLEYYRKQYNQEITDLKQPVLVSQPKRRRGPGGTLPGPAMLIPELCYLTGLTDKMRNDFNVMKDLAVHTRLTPEQRQREVGRLIDYIHKNDNVQRELRDWGLSFDSNLLSFSGRILQTEKIHQGGKTFDYNPQFADWSKETRGAPLISVKPLDNWLLIYTRRNYEAANSLIQNLFKVTPAMGMQMRKAIMIEVDDRTEAYLRVLQQKVTADTQIVVCLLSSNRKDKYDAIKKYLCTDCPTPSQCVVARTLGKQQTVMAIATKIALQMNCKMGGELWRVDIPLKLVMIVGIDCYHDMTAGRRSIAGFVASINEGMTRWFSRCIFQDRGQELVDGLKVCLQAALRAWNSCNEYMPSRIIVYRDGVGDGQLKTLVNYEVPQFLDCLKSIGRGYNPRLTVIVVKKRVNTRFFAQSGGRLQNPLPGTVIDVEVTRPEWYDFFIVSQAVRSGSVSPTHYNVIYDNSGLKPDHIQRLTYKLCHIYYNWPGVIRVPAPCQYAHKLAFLVGQSIHREPNLSLSNRLYYL

SEQ ID NO: 41 - protein, 221 aa, Homo Sapiens
MPLALTLLLLSGLGAPGGWGCLQCDPLVLEALGHLRSALIPSRFQLEQLQARAGAVLMGMEGPFFRDYALNVFVGKVETNQLDLVASFVKNQTQHLMGNSLKDEPLLEELVTLRANVIKEFKKVLISYELKACNPKLCRLLKEEVLDCLHCQRITPKCIHKKYCFVDRQPRVALQYQMDSKYPRNQALLGILISVSLAVFVFVVIVVSACTYRQNRKLLLQ

SEQ ID NO: 42 - protein, 1623 aa, Homo Sapiens
MAAEASKTGPSRSSYQRMGRKSQPWGAAEIQCTRCGRRVSRSSGHHCELQCGHAFCELCLLMTEECTTIICPDCEVATAVNTRQRYYPMAGYIKEDSIMEKLQPKTIKNCSQDFKKTADQLTTGLERSASTDKTLLNSSAVMLDTNTAEEIDEALNTAHHSFEQLSIAGKALEHMQKQTIEERERVIEVVEKQFDQLLAFFDSRKKNLCEEFARTTDDYLSNLIKAKSYIEEKKNNLNAAMNIARALQLSPSLRTYCDLNQIIRTLQLTSDSELAQVSSPQLRNPPRLSVNCSEIICMFNNMGKIEFRDSTKCYPQENEIRQNVQKKYNNKKELSCYDTYPPLEKKKVDMSVLTSEAPPPPLQPETNDVHLEAKNFQPQKDVATASPKTIAVLPQMGSSPDVIIEEIIEDNVESSAELVFVSHVIDPCHFYIRKYSQIKDAKVLEKKVNEFCNRSSHLDPSDILELGARIFVSSIKNGMWCRGTITELIPIEGRNTRKPCSPTRLFVHEVALIQIFMVDFGNSEVLIVTGVVDTHVRPEHSAKQHIALNDLCLVLRKSEPYTEGLLKDIQPLAQPCSLKDIVPQNSNEGWEEEAKVEFLKMVNNKAVSMKVFREEDGVLIVDLQKPPPNKISSDMPVSLRDALVFMELAKFKSQSLRSHFEKNTTLHYHPPILPKEMTDVSVTVCHINSPGDFYLQLIEGLDILFLLKTIEEFYKSEDGENLEILCPVQDQACVAKFEDGIWYRAKVIGLPGHQEVEVKYVDFGNTAKITIKDVRKIKDEFLNAPEKAIKCKLAYIEPYKRTMQWSKEAKEKFEEKAQDKFMTCSVIKILEDNVLLVELFDSLGAPEMTTTSINDQLVKEGLASYEIGYILKDNSQKHIEVWDPSPEEIISNEVHNLNPVSAKSLPNENFQSLYNKELPVHICNVISPEKIYVQWLLTENLLNSLEEKMIAAYENSKWEPVKWENDMHCAVKIQDKNQWRRGQIIRMVTDTLVEVLLYDVGVELVVNVDCLRKLEENLKTMGRLSLECSLVDIRPAGGSDKWTATACDCLSLYLTGAVATIILQVDSEENNTTWPLPVKIFCRDEKGERVDVSKYLIKKGLALRERRINNLDNSHSLSEKSLEVPLEQEDSVVTNCIKTNFDPDKKTADIISEQKVSEFQEKILEPRTTRGYKPPAIPNMNVFEATVSCVGDDGTIFVVPKLSEFELIKMTNEIQSNLKCLGLLEPYFWKKGEACAVRGSDTLWYRGKVMEVVGGAVRVQYLDHGFTEKIPQCHLYPILLYPDIPQFCIPCQLHNTTPVGNVWQPDAIEVLQQLLSKRQVDIHIMELPKNPWEKLSIHLYFDGMSLSYFMAYYKYCTSEHTEEMLKEKPRSDHDKKYEEEQWEIRFEELLSAETDTPLLPPYLSSSLPSPGELYAVQVKHVVSPNEVYICLDSIETSNQSNQHSDTDDSGVSGESESESLDEALQRVNKKVEALPPLTDFRTEMPCLAEYDDGLWYRAKIVAIKEFNPLSILVQFVDYGSTAKLTLNRLCQIPSHLMRYPARAIKVLLAGFKPPLRDLGETRIPYCPKWSMEALWAMIDCLQGKQLYAVSMAPAPEQIVTLYDDEQHPVHMPLVEMGLADKDE

SEQ ID NO: 43 - protein, 443 aa, Homo Sapiens
MRNAIIQGLFYGSLTFGIWTALLFIYLHHNHVSSWQKKSQEPLSAWSPGKKVHQQIIYGSEQIPKPHVIVKRTDEDKAKSMLGTDFNHTNPELHKELLKYGFNVIISRSLGIEREVPDTRSKMCLQKHYPARLPTASIVICFYNEECNALFQTMSSVTNLTPHYFLEEIILVDDMSKVDDLKEKLDYHLETFRGKVKIIRNKKREGLIRARLIGASHASGDVLVFLDSHCEVNRVWLEPLLHAIAKDPKMVVCPLIDVIDDRTLEYKPSPLVRGTFDWNLQFKWDNVFSYEMDGPEGSTKPIRSPAMSGGIFAIRRHYFNEIGQYDKDMDFWGRENLELSLRIWMCGGQLFIIPCSRVGHISKKQTGKPSTIISAMTHNYLRLVHVWLDEYKEQFFLRKPGLKYVTYGNIRERVELRKRLGCKSFQWYLDNVFPELEASVNSL

SEQ ID NO: 44 - protein, 138 aa, Homo Sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 45 - protein, 259 aa, Homo Sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 46 - protein, 407 aa, Homo Sapiens
MPRGHKSKLRTCEKRQETNGQPQGLTGPQATAEKQEESHSSSSSSRACLGDCRRSSDASIPQESQGVSPTGSPDAVVSYSKSDVAANGQDEKSPSTSRDASVPQESQGASPTGSPDAGVSGSKYDVAANGQDEKSPSTSHDVSVPQESQGASPTGSPDAGVSGSKYDVAAEGEDEESVSASQKAIIFKRLSKDAVKKKACTLAQFLQKKFEKKESILKADMLKCVRREYKPYFPQILNRTSQHLVVAFGVELKEMDSSGESYTLVSKLGLPSEGILSGDNALPKSGLLMSLLVVIFMNGNCATEEEVWEFLGLLGIYDGILHSIYGDARKIITEDLVQDKYVVYRQVCNSDPPCYEFLWGPRAYAETTKMRVLRVLADSSNTSPGLYPHLYEDALIDEVERALRLRA

SEQ ID NO: 47 - DNA, 568 bp, Artificial Sequence
  probe derived from KRTAP21-1
ctaacttagtgctttgagtttaaattcttttttcagccactgtggtatgttgattactatattaataatgtgtctcctttatgtgtttttaaatagttacttggtcatgaactatttataagctttggaaacatgatttcatttattctgcaggataacttcaggaagtgggtattaattctcactgtataagtgaagacattaagcaccagagtttaagtagcttgatccactgaaagagattcaactgtgtggaacctatgccgcggagaattgctttctgccattgttattgtccagcagttgccgaatagctctgacctgagcaaagcagtgaaacgcggtgctgttgagtgggttgccactgcacctttttcaactactctagtcttgtggagaaaacaccccttctaaattttttcttcattgaatactctccctcagctccagggtaccatatagagttttcttgtattttagagttactgttttatcaaagtgtaccagtcttttgtattacatttctcctgtctaaannaannntgtggtttctactcattatggatat

SEQ ID NO: 48 - DNA, 417 bp, Artificial Sequence
  probe derived from C12orf37
ttcctcctgaccacaaaaagcacttatactctaggatgactgattccagcccagtggcctggcaagggtgaattacaccttgcatatcacactcttgacatttgtgtgcgctagcataagaattataattgaaacagggatttaagtatctcctctctaggtgcctaccctccttggactcaggtcaaatttattaaaggaagttttgtttctagataggttgtttgaaataaaataacagaatgttcaagtaacacagtgtacctacagcttttaacaaaattgaggacttgggtctcgaaacaatttcctttgattttcaggtattttatctataaaaagggagataaagcattagttcataggacagttatatgtttaaatgtgataatgtatattaaccaccttgcatgtatt

SEQ ID NO: 49 - DNA, 287 bp, Artificial Sequence
  probe derived from WBSCR28
taaggccttgcaagtgaactgcgtggtaaggaagctcctggtacagctgagacgtctgtattggtgggtggagactatgactgccctcacctcctggcacctggcctatctcatcacntggaccacctgcctggcctcccacctgctgcaggctgcctttgagcacacgacccagcnngccgaggcccaggaggttgaaccccaggaggtctcagggtcttccttgctgccctcactgtntgcgtcctcggactcagagtctggaacagttttgccagagcaagaaa

SEQ ID NO: 50 - DNA, 355 bp, Artificial Sequence
  probe derived from RBM46
agcacttgaagacaatggttatagtagatttgattaccaangatcactatctgtanctggagattagaacaattatatgaccagaagcatctaaccattatgtaaaaagaaatgatgagacaaaaagattaagatacaaattttgtgcagtactaaagaaaaagcagtctaccattgtggtccttgaaaataactatagatatttttgttatttgttagacacaaattataattttgttgttaatgtatttaagcattttatagttatgctttgtgtttttgatattctttgtattgttaataacaagtgttatgggtttttaatgttgaaatcatgtgttaatttttgtacttg

SEQ ID NO: 51 - DNA, 381 bp, Artificial Sequence
  probe derived from DKFZp761D1
gcagtacatttcacaatactcagataatcctctcattggaagcaactcaaatgtccaccaactgatggtattacccatgcaatggaatattattcagtcataaaaaggaatgaagtattgaggcatgttacaacatgaaggaactttgaggacagtttgttaatacgctatatactgtgtaattctatttatatgaaatatccagaataggtaaatcattagagactagagacagcagattagtggatgccaggagtttggggaaggaaggggtgggaagaatgggaagtgactgcttaatggatacagggtttctttttggggtgatgaaaaatttctggaactagattgtagtgatggttgcacaacaccgtaagtgta

SEQ ID NO: 52 - DNA, 497 bp, Artificial Sequence
  probe derived from NBPF4
gcagagaaggcccagtgtgtccatccccagtgcggtgatactaggatggtcacttggttaaggaggggtctaggagctctgtcccttgtaaagacatcttatttgtaagtaatttggaaagtggtttgaaatagtataaatatcctgtattctagtgatcttcttcagaacattttatcaccaattaatcaccccgtctgtgtcagttattatatttaagtttgtacattgaaaattgtctatctcaaaatcttaccttatacttgcttttgctggcattcttngtaaaaaagatcattccctgcccaaattttaactttcatccaaaattaattttaatttctttttgctggcattctgttgtgaaaaagaatattctctgccccaattatnactttcatccaaaattaattttagtccatcagttaaaattttaanttttaaatctgtttaattaaaacatttcttgcctctcactctggactattggatttttt

SEQ ID NO: 53 - DNA, 500 bp, Artificial Sequence
  probe derived from ADAM12
gtcttctggactggttttcacattagaagacaattgacaacagttacataattcactctgagtgttttatgagaaagccttcttttggggtcaacagttttcctatgctttgaaacagaaaaatatgtaccaagaatcttggtttgccttccagaaaacaaaactgcatttcactttcccggtgttccccactgtatctaggcaacatagtattcatgactatggataaactaaacacgtgacacaaacacacacaaaagggaacccagctctaatacattccaactcgtatagcatgcatctgtttattctatagttattaagttctttaaaatgtaaagccatgctggaaaataatactgctgagatacatacagaattactgtaactgattacacttggtaattgtactaaagccaaacatatatatactattaaaaaggtttacagaattttatggtgcattacgtgggcattgtctttttagatgcccaaatcct

SEQ ID NO: 54 - DNA, 420 bp, Artificial Sequence
  probe derived from CDNA clone IMAGE5299000
gttgcaatattaccttgggcactgttttgactgcattccacaagtttttatttgtcatgttttaatcaccattcagttcaaaatactttctaatgctccttgttatttcttctttgacccattggttatataaaagtacgttttgctttatttctaaatatgtgaagactttctagatatctcagttatttattactaatttaattttattgtagccagagaaaatacactgtatgtgttcaatctttggaaatttcttgagacttattttttgacccagtatgtttgccctcatgaacattccatattcactagaaaaagaatgtgtgttcaaataatgttggatatagtgttatgcaaatgccactaaatcaagataaatcttttgcactcttgttttttgcctgcttgttctaacaa

SEQ ID NO: 55 - DNA, 544 bp, Artificial Sequence
  probe direved from SOX30
gaaagttttcagtcgtgatttgaggagttaaaaccaaatgcaatttatgtcttcataaaattttgattagtgaaactagagtctggatgtttcattgtaggaatatttaagttattaagtagtttaattttaatggctgaaatttgcatcaacatgtattattattactttatcctggaacatgcaaaatactgaagcctcacagttgtatgtgaggggaaaggggaaataaatctagcatagtgtgatttttattttatctcaggatacattttttaaatgattttttgtttgctttttatgtaatacttatggatgttgtcaatttttgatgtaacattttgaaagtattttgacaactcctagtgaacttggacttggttgctaaatttaacttacactaataaccaattataagttccaaatgtgttttaatggcacctgggtgattcttcagctaaatttagtcatttctgtttctaaatatttttatcattttaaaatattttttttccatttggcatacatcgttctttgttgtaat

SEQ ID NO: 56 - DNA, 493 bp, Artificial Sequence
  probe direved from CDNA clone IMAGE5296886
tgactgtcatatgggaggcactgcaaggtgaaggagacatgtcctcacatttggtctttgggaagcgaagtaggcagaaacccaagtgcaaagtggccatttgttttcctaatgctagataagtacctgatgtgttggaagggtggagtagaactagaaactcctggaagttcttttaagaacgaatgcaggacctcatcagcccgagtctttaaataagtcacacatttttggtcaccacattctggaagggggacaaaaagatctgtcacagacaaaatttggagatatttaaggctttgttgactttggcaaatgcttgattatagttctgtggtctgtttgttttctagagtctccatgaattgcaagggaaaacaagtggtacactggtatttctatttgataacctgttctctttggaattccggttagaaatcaaaggcttacttgagttttaatgctttcgggacttcaaatgcttttctatctt

SEQ ID NO: 57 - DNA, 375 bp, Artificial Sequence
  probe derived from ASZ1
gataacactggaatgggcttctccccagaattttacttcagtttgtgaagaattggttaataatgttgaagatttgagtgaaaaggtctgcaaactaaaagacctaattcaaaagttgcaaaatgaacgggaaaatgatccaactcatatacaattaagggaagaagtatctacatggaatagtagaattttgaagaggacagctattaccatatgcggattcggttttcttcttttcatttgcaagctaactttccagaggaaataatccaaatatttgttttaagttatatatacacatcttattgaaccatccaaaaatcattgcttttaactgtttgcagcacaatataactttacattctttgccaatat

SEQ ID NO: 58 - DNA, 504 bp, Artificial Sequence
  probe derived from SLC2A14
agagaccgagtgaacctaccttcatttcaggagggattggccgcttggcacatgacaactttgccagcttttcctcccttgggttctgatattgccgcactagaggatataggagaggaaaagtaaggtgcagttgccccaacctcagacttaccaggaagcagatacatntgagtgtggaagncngagggngtttatgtaagagcaccttcctcacttccatacagctctacgcggcaaattaacttgagttttatttatctnatcctctggtttaattacataaatatttattttttaagtgtaattttgccaaataataanancagnaaggaaattganantagagggaggtgtttaaagagaggttatagagtaaaagatttgatgctggagaggttaaggtgcaataagaattcagggagaaatgttgttcattattggagggtaaatgatgtggtgcctgaggtctgtacattacctcttaacaatttctgtccttca

SEQ ID NO: 59 - DNA, 313 bp, Artificial Sequence
  probe derived from COX8C
ccctgtctgccgcggaaatggctgttggacttgtggtgttttttacnaccttcttaacaccagctgcatatgtgctaggcaacctgaagcagttcagaaggaattagatggaagatgatgttgaacagctgttaacgtccaaaaaactttcagaaaaagctgtgtttttgttaacgagcaaaattgcctagttgagttgatgcaaccattgtggtattcactttcctcatgtttatgatgaatattttgcacttttttagtactgtgcattatatagatgtatagtcaaaaatgttctgcttaagtgttaaat

SEQ ID NO: 60 - DNA, 353 bp, Artificial Sequence
  probe derived from TKTL2
gactacaaatatctgactgagctggggattaaagtagaggtaactgtatcttaaatgagtatgatttccttgtaagttaaaaaaattgaaatttaattgtagacttcaatagtccaagttttgaaggatgtttgagcttttgtataatgccatttatacctgcagttttacagataatgtttgactgcagttgccttggaaattcctccaaagtttgccttcatctctcctctacagtttggaggtgatggtgcagcagtggaacatctcttgatgcaccacactacttgtgttctgtgaagtgatgaaagtataactggttctagtttgcacactacacacatagttttgtg

SEQ ID NO: 61 - DNA, 364 bp, Artificial Sequence
  probe derived from TPTE2P2
ctgaggttctattcatggtgagcaagtcttttttttngtttgtttcttcaagctctaacaagggtgcctactacatggcttttcagttancnccaaaataanatgtnacaattttttttnctattcttaggctttatctacaaagaaatgaattggataatcttcataaacaaaaaacntggaaaatttatcaaccagaatatgcagtagaganatattttnatgagaaatgacttaagttatgttgtaactggtagctgattaagtatagttcccngcaccccttctgggaaagaattatgttctttctaaccctgccacatagttatatgttctaaatcttccttgctggtacatctatatt

SEQ ID NO: 62 - DNA, 217 bp, Artificial Sequence
  probe derived from C9orf11
gtatttaaattactacactgtattcatacctggagaatatcattatgtgacttanaaanntnnntnnnnntncnctnnncnnnncatnannnnanagcnnnnnnannnnntttatactgctaccttttaaagtcactaaaatcttttaaatggtgaatgtctaagagtcagtcttcaggtatacctgacaaaggatgagaaatgagcttttgtagaa

SEQ ID NO: 63 - DNA, 546 bp, Artificial Sequence
  probe derived from PIWIL1
attcaccggcttccttattttatatgtaaaaattaagattttatattttatcttcttgtttctcatagatattttgtgagcatttttttgtttattttgaagaaatgtggataagatacttggtagtataaaacagactctctgagangtatttgaaatgtgtttggagatttacttaaacgtactttcaggagtgagcaagtcctacttataaacctatattaactttatttttgagatacctgttttgaatttaaaggagataagaggcgtaaagtaggatgctcactacaaccataggtggggtttcagctcatatcttaaagataaaaggtactattatataacctatacacaagatacaggagaaaatatgcttgatttttatttggcaggggggctaggttgtatgggagtaaaaaaaacattgaaaatttttaaattgtccaaagaaacattttaagactctttaacaaaaaaggccatgagtaaatctctatattaacattactatttattttgttttggaactgggacatgattcta

SEQ ID NO: 64 - DNA, 521 bp, Artificial Sequence
  probe derived from C19orf41
ggccgtgcgtgatgggcatggaggggcctttcttccgggactacgcgctgaacgtgtttgtggggaaagtggagacaaatcaactggaccttgtggcgtcctttgtcaagaaccaaacgcagcacttaatgggtaactctctgaaagatgagcctctgctggaagagctggtgaccctcagggcgaatgtgatcaaggaattcaagaaagttttaatttcatatgaattaaaagcctgcaaccccaaactttgccgcttgctaaaagaagaggtgttggactgtttacattgccagaggatcactcccaagtgtatccacaaaaagtactgctttgtcgaccggcaaccccgcgtggccctgcagtaccagatggacagcaaatacccgaggaaccaggcgctgttgggcatcctcatttctgtgtctctggctgtctttgtcttcgtggtcatcgtggtctcggcttgtacatacagacaaaaccgaaaactcctgctgcagtaggacggtggtttgggg

SEQ ID NO: 65 - DNA, 486 bp, Artificial Sequence
  probe derived from RNF17
gaatttaatcctttatctatcttagtacaatttgttgattatggatcaactgcaaagctgacattaaacagactgtgccaaattccttctcatcttatgcggtatccagctcgagccataaaggttctcttggcagggtttaaacctcccttaagggatctaggggagacaagaataccatattgtcccaaatggagcatggaggcactgtgggctatgatagactgtcttcaaggaaaacaactctatgctgtgtccatggctccagcaccagaacagatagtgacattatatgacgatgaacagcatccagttcatatgccgttggtagaaatggggcttgcagataaagatgaataagtgcctaagtgtatacagtgagagcatctatagaagcctagaagaattctgttatgtttagactatgtcttatctttagactatttcaggcttaattttcctaacttgttcagccctagtgcttta

SEQ ID NO: 66 - DNA, 306 bp, Artificial Sequence
  probe derived from GALNTL5
ggaactttcactaaggatctggatgtgtggaggccaactctttnataatcccctgctctcgagtaggacatatcagtaagaaacaaactggaaaaccttctacaatcatcagtgctatgacacataactacctaagactggtgcacgtttggctggatgaatataaggagcagttttttcttcgaaagcctggtctgaaatatgtcacctacggaaatattcgcgagcgtgttgagttaaggaaacgactgggttgcaagtcatttcagtggtatttggataatgtcttcccagagttggaggcat

SEQ ID NO: 67 - DNA, 511 bp, Artificial Sequence
  probe derived from KISS1
aagcctcaaggcacttctaggacctggctcttctcaccaagatgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagccgccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctggggcgcaggtgcggggcagtgaacttcagaccccaaaggagtcagagcatgcgg

SEQ ID NO: 68 - DNA, 526 bp, Artificial Sequence
  probe derived from IGFBP1
aataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtcgataat

SEQ ID NO: 69 - DNA, 279 bp, Artificial Sequence
  probe derived from MAGEB6
tcacattcatggctgtttaaccaatctgaaagttacggtttgggaattaataaaacaaagtcatacaacacattttctttgtaattgagaactagataacatggtaacagagaattgattttcatatgaatcttaacnccacagtaaaatagttgacatcataatangaagagaaagaaaaggaaaaacagaaatgtaaaagttgtttaattcttggtttgcctaattcgttttcctatttcttttcatacaaataaaggatacctggatttatttagg